(19) United States
(12) Patent Application Publication
(10) Pub. No.: US 2024/0161878 A1
(43) Pub. Date: May 16, 2024

(54) COMPUTER MODEL BASED HEAT TRANSFER FLUID LIFE AND QUALITY ESTIMATIONS

(71) Applicant: Eastman Chemical Company, Kingsport, TN (US)

(72) Inventors: Mark Gregory Brucks, Palatine, IL (US); Conrad E. Gamble, Heflin, AL (US)

(73) Assignee: Eastman Chemical Company, Kingsport, TN (US)

(21) Appl. No.: 18/552,465

(22) PCT Filed: Mar. 29, 2022
(86) PCT No.: PCT/US2022/022264

§ 371 Date: Sep. 26, 2023

Related U.S. Application Data
(60) Provisional application No. 63/200,832, filed on Mar. 31, 2021.

(51) Int. Cl. G16C 60/00; G16C 20/70

(52) U.S. Cl. CPC G16C 60/00; G16C 20/70; F28F 2200/00

(57) ABSTRACT

Various embodiments are directed to improving the accuracy of existing hardware-based fluid quality measurement systems and particular computer applications. For instance, some embodiments improve the accuracy of these technologies by generating, via a computer model, an estimate of a fluid life for a heat transfer fluid and/or a score that indicates a quality of the heat transfer fluid, among other things. Additional embodiments also improve human-computer interaction, user interfaces, and computer resource consumption relative to existing technologies.

### BACKGROUND OF THE INVENTION

Generally, certain systems may utilize a heat transfer fluid where cooling and/or heating of one or more system components is required to obtain or maintain a particular temperature. For instance, these fluids can act as an intermediary to receive or provide thermal energy to one portion of a process or system and transfer and/or store thermal energy to another portion of the process or system. When heat transfer fluids are not performing up to expectations, there can be detrimental impacts to the system, such as adverse effects to machine or equipment life, and/or system production rates. Faulty heat transfer fluids can also consume unnecessary resources in troubleshooting.

Certain conventional technologies may assess a heat transfer fluid using sensor-based systems, fluid quality measurement systems, and/or certain computer applications. However, such conventional technologies are inaccurate and/or fail to provide meaningful information. Further, certain conventional processes can negatively affect the user experience and consume unnecessary computing resources, among other things.

### SUMMARY OF THE INVENTION

Particular embodiments of the present disclosure include a computer-implemented method, a non-transitory computer storage medium, and a system that are directed to improving the accuracy of existing hardware-based fluid quality measurement systems and particular computer applications. For instance, some embodiments improve the accuracy of these technologies by generating, via a computer model, an estimate of a fluid life (also referred to herein as a “fluid life expectancy” (FLE), life remaining, or fluid life) for a heat transfer fluid and/or a score that indicates a quality or condition of the heat transfer fluid (also referred to herein as a “fluid condition score” (FCS), “fluid quality score”, and “fluid analysis score”), among other things. In some embodiments, historical data is accessed from a data store in computer memory. This historical data may correspond to an extensive sample analysis archive that indicates various heat transfer fluid samples and their parameters (e.g., high and low molecular weight compounds, moisture content value, acid number value, insoluble solids value, soluble solids value viscosity, opacity, color, conductivity absorbance, etc.) and patterns or other learned relationships between historical heat transfer fluid samples that can be used for the estimation of the fluid life or quality. For example, a pattern can be detected that when a key parameter value exceeds a particular threshold, then the fluid quality is poor or there is no fluid life remaining. Accordingly, when a new heat transfer fluid is analyzed, and the key parameters exceeds the particular threshold for that parameter, then it can be predicted that the fluid quality is poor.

Additional embodiments also improve human-computer interaction, user interfaces, and the user experience (e.g., via less drilling, historical graphing, trend lines, and more intuitive user interface features) and computer resource consumption (e.g., memory consumption and CPU bottlenecking) relative to existing technologies. Other advantages, embodiments, improvements and the like are described herein.

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE DRAWING

The present technology is described in detail below with reference to the attached drawing figures, wherein:

FIG. 1 is a block diagram of an illustrative system architecture, according to some embodiments.

FIG. 2 illustrates a screenshot of an example user interface, according to some embodiments.

FIG. 3 illustrates a screenshot of an example user interface, according to some embodiments.

FIG. 4 illustrates a screenshot of an example user interface, according to some embodiments.

FIG. 5A illustrates a screenshot of an example user interface, according to some embodiments.

FIG. 5B illustrates a screenshot of an example user interface, according to some embodiments.

FIG. 6 illustrates a screenshot of an example user interface, according to some embodiments.

FIG. 7 illustrates a screenshot of an example user interface, according to some embodiments.

FIG. 8A illustrates a screenshot of an example user interface, according to some embodiments.

FIG. 8B illustrates a screenshot of an example user interface, according to some embodiments.

FIG. 9A illustrates a screenshot of an example user interface, according to some embodiments.

FIG. 9B illustrates a screenshot of an example user interface, according to some embodiments.

FIG. 10 illustrates a screenshot of an example user interface, according to some embodiments.

FIG. 11 is a schematic diagram illustrating how a decision statistic is generated using one or more machine learning models, according to some embodiments

FIG. 12 is a schematic diagram of an example visualization of vector space that illustrates various clusters or classes of feature vectors, according to some embodiments.

FIG. 13A is a schematic diagram of a cluster analysis visualization at a first time, according to particular embodiments.

FIG. 13B is a schematic diagram of a cluster analysis visualization at a second time subsequent to the first time illustrated in FIG. 13A.

FIG. 14 illustrates an example random forest learning model, according to particular embodiments.

FIG. 15A is a schematic diagram of an example exponential smoothing forecast model table, according to some embodiments.

FIG. 15B is a schematic diagram of an example time series graph associated with the table of FIG. 15A, according to some embodiments.

FIG. 16A is a schematic diagram illustrating how a heat transfer fluid quality score is generated, according to some embodiments.

FIG. 16B is a chart illustrating different parameter thresholds, according to some embodiments.

FIG. 17 is a flow diagram of an example process for training a machine learning model using heat transfer fluid samples, according to some embodiments.

FIG. 18 is a flow diagram of an example process for generating one or more estimates associated with a heat transfer fluid, according to some embodiments.

FIG. 19 is a flow diagram of an example process for estimating a life remaining of a heat transfer fluid, according to some embodiments.

FIG. 20 is a flow diagram of an example process for modifying a fluid life expectancy and/or a fluid condition score based on a heat transfer fluid system being serviced, according to some embodiments.

FIG. 21 is a block diagram of a computing environment in which aspects of the present technology are implemented within, according to some embodiments.

FIG. 22 is a block diagram of a computing device in which aspects of the present disclosure are implemented within, according to some embodiments.

### DETAILED DESCRIPTION OF THE INVENTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different components of methods employed, the terms should not be interpreted as implying any particular order among or between various steps herein disclosed unless and except when the order of individual steps is explicitly described.

Existing hardware and sensor-based systems can be inaccurate or fail to estimate a fluid life or quality of a heat transfer fluid. For example, various fluid quality measurement systems measure the fluid quality of oil used in machinery. These systems use a filter device, sensor, and electrodes to sample the fluid to determine (e.g., via H₂O levels, soot, acid contamination) when to make an oil change due to a change in the quality of the oil. Other technologies are directed to Infrared (IR)-absorption sensor systems for the determination of engine oil deterioration via capturing the oxidation index to detect soot, water, and glycol. Yet other systems use chemical-based reaction mediums and equipment to detect wear in a metalworking fluid. However, with each these existing technologies, there are only a limited number of parameters (e.g., soot, glycol) that any one of these sensor systems can measure and so predictions are based on only a limited amount of information. Standard oil analysis labs lack specific expertise in interpretation of the combined test results with respect to heat transfer fluid performance. Accordingly, predictions are inaccurate. There are several parameters together that are important for assessing heat transfer fluid (e.g., moisture content, carbon residue, etc.) but which none of these hardware-based sensors measure.

These existing hardware and sensor-based systems are also costly and time consuming to operate. Moreover, they also provide no predictions or estimations for the fluid life of a heat transfer fluid. Rather, these technologies provide only real-time assessments of the current state of fluids, which can cause detrimental effects. For example, a user may sample a fluid at a first time with real-time information indicating that the fluid is of high quality. However, with no indication of when the user needs to sample again or how long the fluid will last, the user may, at a second subsequent time, sample the fluid once again. The real-time information may indicate that the user needs to change fluid. However, by the time this indication is received by the user, the fluid quality may be so poor that there may have already been detrimental wear or other irreversible effects done to the machinery carrying the fluid. Therefore, these hardware or sensor-based fluid quality measurement systems are detrimental to heat transfer fluid systems because they provide no heat transfer fluid life predictions.

Existing technologies also unnecessarily consume computer resources. For example, some existing computer applications use 2D and 3D maps together to calculate a rate of degradation of oil based on a determined engine variable and operating parameters. These two-dimensional maps include several multiplier values based on oil temperature. These three-dimensional maps contain several operation parameters that are simultaneously considered to determine multiplier values based on rate of fuel consumption of an engine and rotational speed of the engine. However, these maps, especially the three-dimensional maps, are memory intensive and cause CPU (or GPU) bottlenecking, latency, and throughput issues. This is due to the large data storage requirements and multiple data units that are not able to meet the need of the simultaneous processing and graphics data processing requirements. For example, a processor may outpace a graphics card that is busy processing image data of the three-dimensional map, causing bottlenecking.

Existing technologies also negatively affect human-computer interaction, user interfaces, and the overall user experience. For example, various existing spreadsheet computer applications require tedious manual user input in order to make any assessment for fluids. For example, a user may have to manually input a fluid identifier and several attributes known about the fluid, such as type of fluid, age of fluid, and last install. These attributes are stored to computer memory and then the application can perform some basic calculation (e.g., a BASE function) to assess certain attributes. But these technologies do not predict the heat transfer fluid life or quality of a heat transfer fluid and they are very arduous to use due to the manual data entry requirements. In another example, existing user interfaces negatively affect user navigation by requiring the user to drill down, scroll through, query, or browse to obtain various pieces of information regarding a fluid to obtain relevant information. Further, existing user interfaces are non-intuitive to use.

Various embodiments of the present disclosure provide one or more technical solutions to the technical problems described above, as well as other problems. In operation, various embodiments of the present disclosure are directed to generating, via a computer model, an estimate of a fluid life (e.g., life remaining) for a heat transfer fluid and/or a score that indicates a quality of the heat transfer fluid, among other things. In some embodiments, historical data is accessed from a data store in computer memory. This historical data corresponds to an extensive sample analysis archive that indicates various heat transfer fluid samples and one or more associated parameters (e.g., high and low molecular weight materials, moisture content value, acid number value, insoluble solids value, conductivity, soluble solids, absorbance, etc.) and patterns or other learned relationships between historical heat transfer fluid samples that can be used for the estimation of the fluid life or quality. For example, a pattern can be detected (e.g., via a machine learning model) between historical heat transfer fluid samples of type A that a heat transfer fluid had X years remaining, when the certain parameters such as acid number value was at Y. Accordingly, when a new heat transfer fluid of type A is received, the computer model can estimate that given that the key parameter was at or near Y (the same value as the historical samples), it will likely have X years remaining based on the patterns detected and observations of the historical data. These estimations, as well as other information, are also indicated at user interfaces.

Various embodiments of the present disclosure improve the accuracy of existing technologies. As described above, existing hardware and sensor-based systems fail (or are inaccurate) to estimate a fluid life or quality of a heat transfer fluid. Unlike these technologies, various embodiments use different and more parameters for a single estimation associated with a heat transfer fluid. In certain embodiments, there are several parameters together that are important for assessing heat transfer fluid (e.g., high and low molecular weight components, moisture content value, conductivity, soluble solids, absorbance, acid number value, insoluble solids value, etc.), which can be used to make an estimation for heat transfer fluid life or heat transfer fluid quality. However, existing fluid quality measurement systems and other technologies fail to use these parameters for their predictions and are limited to only a few parameters based on the limited functionality of their hardware or sensor systems. Accordingly, various embodiments of the present disclosure are more accurate relative to existing technologies.

Moreover, particular embodiments of the present disclosure are not as costly and time consuming to operate relative to existing technologies because there are no installed bulky hardware requirements (e.g., filter device and electrodes). Moreover, some embodiments of the present disclosure improve existing technologies by estimating the fluid life of a heat transfer fluid. As described above, these existing hardware and sensor-based systems provide only real-time assessments of the current state of fluids without a predicted heat transfer fluid life, which can be detrimental. By estimating an end life or life remaining of a heat transfer fluid, advanced notice is given to users to change or otherwise treat heat transfer fluid. Additionally this advanced notice allows users time to develop and gain approvals for expenditures prior to an urgent need. For example, using the illustration above, a user may sample a fluid at a first time with near-real-time information indicating that the heat transfer fluid is of high quality. At or near the first time, a user interface can indicate that the heat transfer fluid likely has only 1 year left before it needs a change. Accordingly, instead of the user arbitrarily testing the heat transfer fluid a second time, which may be too late, the user can use the heat transfer fluid life estimation information to perform another test (or change/treat the fluid) at a second time well before the 1 year period is up. Accordingly, the heat transfer fluid will not be poor so there will be no detrimental wear or other irreversible effects done to the machinery carrying the fluid.

Some embodiments of the present disclosure also improve computer resource consumption, relative to existing technologies. As described above, some existing computer applications use 2D and 3D maps together to calculate a rate of degradation of oil based on a determined engine variable and operating parameters. Particular embodiments do not require the usage or storage of these maps. In other words, these embodiments do not store maps that require multiple data units to meet the need of the simultaneous processing and graphics data processing requirements. In this way, for example, a processor will not outpace a graphics card that is busy processing image data of the three-dimensional map, since there are no three-dimensional maps to analyze. Accordingly, these embodiments are not as memory intensive and do not cause CPU (or GPU) bottlenecking, latency, and throughput issues.

Various embodiments of the present disclosure also improve user human-computer interaction and the overall user experience relative to existing technologies. For example, instead of a user may having to manually input a fluid and several attributes known about the fluid in order to make a basic calculation assessment for the fluid, as is common with existing spreadsheet computer application, various embodiments automatically (without manual input) predict the heat transfer fluid life or quality of a heat transfer fluid based on new rules (e.g., specific parameter thresholds) that existing technologies do not use. Accordingly, the user does not have to arduously perform manual data entry to obtain the desired predictions, which improves human-computer interaction and the user experience.

Various embodiments also improve existing user interfaces relative to existing user interfaces for assessing fluids. As described above, existing user interfaces require the user to drill down, scroll through, query, or browse to obtain various pieces of information regarding a fluid to obtain relevant information. However, various embodiments of the present disclosure provide intuitive user interfaces and improve user navigation by reducing the amount of arduous drilling, scrolling, querying, or browsing required to obtain relevant information. For example, in some embodiments, a single user interface page is caused to be presented, where the single page includes a fluid life estimation, a fluid quality or condition score, and an indication of whether several parameter values fall within a normal range. Accordingly, for example, instead of the user having to drill down various pages to receive information about various fluid attributes and predictions (as is required in some existing technologies), various embodiments provide the fluid life estimation, the fluid quality score, and all parameter information to a single page. This negates any need to perform other drilling or other extensive manual user input. Additionally, various embodiments of the present disclosure include other user interface features, such as heat map indicators, dials, and other visual features to allow users to quickly assess their heat transfer fluid. This may be especially helpful if English is not the first language of a user.

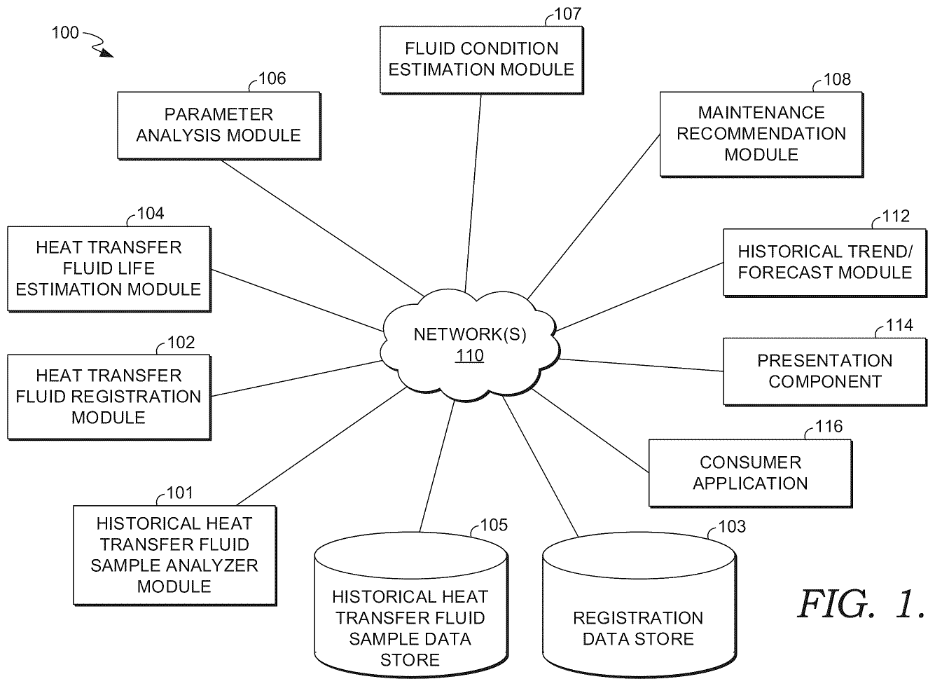

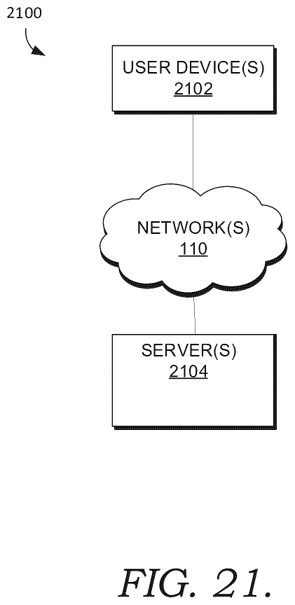

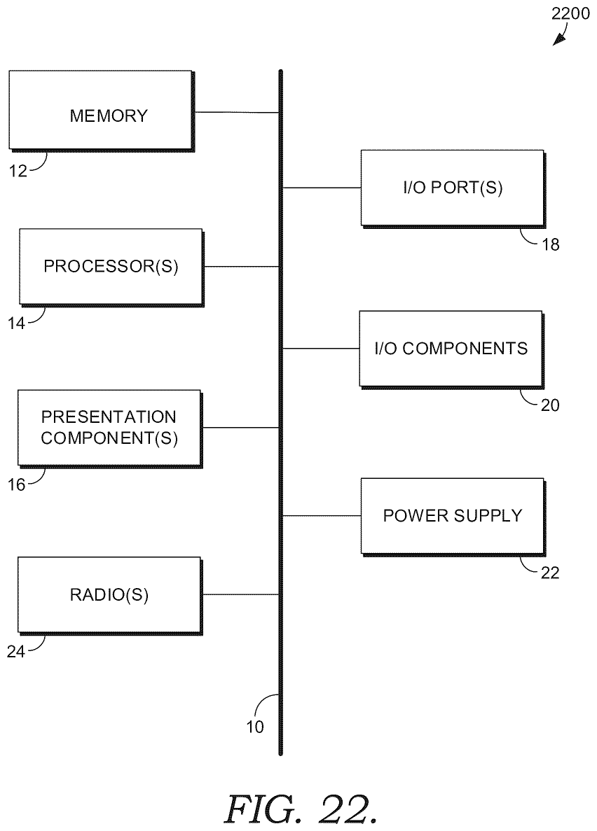

FIG. 1 is a block diagram of an illustrative system architecture 100 in which some embodiments of the present technology may be employed. Although the system 100 is illustrated as including specific component types associated with a particular quantity, it is understood that alternatively or additionally other component types may exist at any particular quantity. In some embodiments, one or more components may also be combined. It is also understood that each component or module can be located on the same or different host computing devices. For example, in some embodiments, some or each of the components within the system 100 are distributed across a cloud computing system (e.g., the computer environment 2100 of FIG. 21). In other embodiments, the system 100 is located at a single host or computing device (e.g., the computing device 2200 of FIG. 22). In some embodiments, the system 100 illustrates executable program code such that all of the illustrated components and data structures are linked in preparation to be executed at run-time.

System 100 is not intended to be limiting and represents only one example of a suitable computing system architecture. Other arrangements and elements can be used in addition to or instead of those shown, and some elements may be omitted altogether for the sake of clarity. Further, many of the elements described herein are functional entities that may be implemented as discrete or distributed components or in conjunction with other components, and in any suitable combination and location. For instance, the functionality of system 100 may be provided via a software as a service (SAAS) model, e.g., a cloud and/or web-based service. In other embodiments, the functionalities of system 100 may be implemented via a client/server architecture.

The system 100 is generally directed to estimating a fluid life (e.g., life remaining) or quality score of one or more heat transfer fluids, among other things. The system 100 includes a historical heat transfer fluid sample analyzer module 101, a heat transfer fluid life estimation module 102, a parameter analysis module 106, a fluid condition estimation module 107, a maintenance recommendation module 108, a historical trend/forecast module 112, a presentation component 114, a consumer application 116, a historical heat transfer fluid sample data store 105, and a registration data store 103, each of which are communicatively coupled to the network(s) 110. The network(s) 110 can be any suitable network, such as a Local Area Network (LAN), a Wide Area Network (WAN), the internet, or a combination of these, and/or include wired, wireless, or fiber optic connections. In general, network(s) 110 can represent any combination of connections (e.g., APIs or linkers) or protocols that will support communications between the components of the system 100.

The historical heat transfer fluid sample analyzer module 101 is generally responsible for analyzing historical heat transfer fluid samples (or corresponding feature vectors) stored in the data store 105. Such analyzation is done to detect one or more patterns, relationships, thresholds, trends, and/or other insights among the historical heat transfer fluid samples. In some embodiments, such historical heat transfer fluid samples represent a real (or non-synthetic) customer data archive (e.g., of over 50 years), where previous heat transfer fluid users have sent samples in for condition monitoring of their respective heat transfer fluids. For instance, the data store (e.g., a relational database) 105 may include parameter value measurements, as well as heat transfer fluid quality scores, end life estimations, parameter scores (e.g., whether a parameter value is normal), or other predictions associated with a corresponding heat transfer fluid at a particular snapshot or point in time. In this way, various patterns, thresholds, or relationships can be identified, as described in more detail below. This historical data is also presented together with fluid system historical actions for easy understanding and analysis.

Heat transfer fluids are generally heat-stable fluids that provide indirect transfer of heat to one or more systems or processes. In some embodiments, heat transfer fluids are preferably used for adding or removing heat to/from a process by non-contact transfer. Alternatively, however, in some embodiments, heat transfer fluids are used for adding or removing heat to/from a process by contact transfer. In some embodiments, a “heat transfer fluid” (or “heat transfer fluid sample”) as described herein refers to a single heat transfer fluid or a mixture of two or more heat transfer fluids, such as two different heat transfer fluid types. Example processes and/or systems that can utilize heat transfer fluids include but are not limited to one or more aspects of oil and gas processing, natural gas purification, refining, asphalt processing and storage, plastics and polymer manufacturing, synthetic fiber manufacturing, plastic molding, chemical processing, including pharmaceutical manufacturing specialty chemical manufacturing, concentrated solar power plants, biofuel manufacturing, food and beverage processing, medium-density fiberboard manufacturing, and desalination. In certain embodiments, the heat transfer fluids can be synthetic and/or compounds or compositions. Certain example heat transfer fluids include, but are not limited to, various organic and synthetic aromatic fluids, including ethers, molten salts, and glycol-water mixtures, modified terphenyl fluids, terphenyl and/or quaterphenyl fluids, diphenyl oxide (DPO)/biphenyl constituted fluids, isopropyl biphenyl fluid mixtures, alkyl substituted aromatic fluids, and mineral oils. In various embodiments, the heat transfer fluid can include any or all of the commercially available heat transfer fluids sold under the THERMINOL® brand and MARLOTHERM® brand heat transfer fluids by Eastman Chemical.

A “parameter” as described herein refers to a particular attribute, feature, element, or other characteristic that makes up, is measured from, or is otherwise associated with a heat transfer fluid. For example, a parameter can be or include a high boiler, low boiler, moisture control, acid number, conductivity, soluble solids, absorbance, acid number value, insoluble solids value, or other attributes. Specific parameters are described in more detail below. A “parameter value” (also described herein as “values” associated with parameters) represent the particular numbers or specific parameter information. For example, a particular heat transfer fluid can include an “acid number” parameter with a value of 0.50 mg KOH/g. Particular parameter values are associated with heat transfer fluid quality and heat transfer fluid life, as described in more detail below.

In an illustrative example of how the historical heat transfer fluid sample analyzer module 101 can function, it can determine that several heat transfer fluid samples that were determined to be in “fair” condition had a viscosity parameter value between X and Y (denoted herein as letters for simplicity, but is typically indicative of some real number, such as an numerical). Responsively, the historical heat transfer fluid sample analyzer module 101 can tag or flag X and Y as the range of parameter values that are acceptable for a heat transfer fluid being estimated to be in “fair” condition based on analyzing these past historical heat transfer fluid samples.

In some embodiments, the historical heat transfer fluid sample analyzer module 101 uses one or more computer models to determine patterns or relationships among the historical heat transfer fluid samples. For example, some embodiments use a machine learning model (e.g., a deep neural network) to learn heat transfer fluid patterns based on weighting for non-linear node activation. For example, various models can establish a ground truth using labels (e.g., a “fair” fluid condition score or “normal” acidic value number) on various heat transfer fluid samples, each with multiple parameter values. Embodiments can then responsively train on and learn which parameter values are associated with the ground truth. For example, after inputting 30 historical heat transfer fluid samples into a neural network, embodiments can learn that a parameter most indicative of the high quality or a fluid life is a particular high boilers value. Accordingly, these models can weight and activate the corresponding neural network node connections associated the high boiler value parameter or feature to make future estimations. Machine learning models in the context of these embodiments are described in more detail below.

The heat transfer fluid registration module 102 is generally responsible for deriving and storing, in computer memory (e.g., to the registration data store 103), information about a user and/or a new heat transfer fluid (e.g., a heat transfer fluid that has not been stored to the data store 105 and has not been analyzed via the historical heat transfer fluid sample analyzer module 101). In other words, the new heat transfer fluid is excluded or not a part of any previous analysis. In an illustrative example, a user may request a heat transfer fluid sampling kit to provide a sample of heat transfer fluid. After the heat transfer fluid sample is received from the user, the sampled fluid various parameter values are determined such that the heat transfer fluid registration module 102 stores the parameter value information to the registration data store 103. In some embodiments, the heat transfer fluid registration module 102 additionally or alternatively generates other information associated with a user, such as user name or other account information (e.g., billing information, address, etc.), different plant IDs or heat transfer fluid systems of a user, fluid age of the new heat transfer fluid, and the like, which is described in more detail below.

The heat transfer fluid life estimation module 104 is generally responsible for estimating a fluid life for a heat transfer fluid. In some embodiments, a “fluid life” as described herein refers a life remaining for a heat transfer fluid. For example, the heat transfer fluid life estimation module 104 can estimate that there are “4 years remaining” until a heat transfer fluid life is substantially compromised for further use in a heat transfer fluid system. Alternatively or additionally, in some embodiments a fluid life refers to a point in time at which the heat transfer fluid will expire. For example, the heat transfer fluid life estimation module 104 can estimate that a heat transfer fluid life will expire on February 2023, and cause display of an identifier of “February 2023,” which is indicative of the heat transfer fluid being suitable for use until that time.

In some embodiments, the heat transfer fluid life estimation module 104 performs its functionality in response to the functionality performed by the heat transfer fluid registration module 102. For example, in response to registering a user's new heat transfer fluid sample (e.g., storing its parameter information in the data store 103 and associating it with various user information, such as plant ID, etc.), the heat transfer fluid registration module 102 programmatically calls the heat transfer fluid life estimation module 104, which triggers a heat transfer fluid life estimation for the new heat transfer fluid sample.

In some embodiments, the heat transfer fluid life estimation module 104 performs its functionality based on the functionality performed by the historical heat transfer fluid sample analyzer module 101. For example, the heat transfer fluid life estimation module 104 may compare the new heat transfer fluid sample and its parameters against a set of dynamic rules and parameter value thresholds contained in the historical heat transfer fluid sample data store 105, which were generated based on determined patterns of the historical heat transfer fluid samples by the historical heat transfer fluid sample analyzer module 101.

In an illustrative example, the historical heat transfer fluid sample analyzer module 101 may have identified a pattern that the average or median time of all historical heat transfer fluids analyzed had 5 years remaining of an end life when a contamination parameter was at a certain value P (or within a threshold of P). Responsively, embodiments can generate conditional rules, objects, programming, or other logic (inside the data store 105) that states “if a contamination parameter is less within a threshold value of P, then increment a fluid life estimation for around 5 years remaining.” Accordingly, a new heat transfer fluid sample may be read having a value that exceeds value P within the threshold. Responsively, the heat transfer fluid life estimation module 104 can read the conditional rules or other logic and determine that the fluid life estimation will be around for 5 years. It is understood that this example represents simplified logic. Other parameters or a combination of parameters and weighting may additionally or alternatively be utilized. For example, using the illustration above, the 5-year estimation can be modified to a 3-year estimation based on other parameters that are sampled and compared against other conditional rules or logic, which is described in more detail below.

In some embodiments, the heat transfer fluid life estimation module 104 uses one or more computer models to make its predictions. For example, the heat transfer fluid life estimation module 104 may use the same machine learning model used by the historical heat transfer fluid sample analyzer module 101 to make predictions. In an illustrative example, a deep neural network may learn weights corresponding to neural network nodes indicating that when a feature vector representing a heat transfer fluid sample and its parameters are within a particular distance (e.g., a Euclidian distance or Cosine distance) from a ground truth labeled “5 years remaining”, then embodiments can predict that the heat transfer fluid sample has 5 years remaining. These machine learning models are described in more detail below.

The parameter analysis module 106 is generally responsible for determining whether one or more parameter values of a heat transfer fluid meet, exceed, or are within one or more thresholds (e.g., predetermined parameter values, levels, or limits). In some embodiments, such thresholds correspond to whether parameter values (e.g., of a new heat transfer fluid) fall within a “normal/acceptable” range, a “marginal/marginal/nominal/actionable” range meaning that some action (e.g., refill or treat) will soon need to be taken with respect to the heat transfer fluid, or an “action/beyond limit” range meaning that some action immediately needs to be taken with respect to the heat transfer fluid. In some embodiments, when the parameter test results all fall into a “acceptable” range, the fluid is likely in good condition. If one or more of the tests fall into the “marginal” area, the system operator may consider taking appropriate action such as venting, filtration, etc. When the results fall in the “beyond limit” area, taking action to return the fluid to a more normal condition either by appropriate fluid treatment or complete drain and refill is more of a necessity.

In some embodiments, the one or more thresholds are determined based on patterns, relationships, or other insights derived by the historical heat transfer fluid sample analyzer module 101. For example, it can be determined that several viscosity parameters of several heat transfer fluid samples that were in an “acceptable” range, had a viscosity parameter value between X and Y. Responsively, the historical heat transfer fluid sample analyzer module 101 can tag or flag X and Y as the range of parameter values (or some other aggregated value, such as the mean value) that are acceptable for the viscosity parameters to be determined to be in a “acceptable” condition based on analyzing these past historical heat transfer fluid samples.

Accordingly, for example, when the parameter analysis module 106 analyzes a new heat transfer fluid's viscosity parameter value, if the range is between X and Y, the value can be determined to be in the acceptable range.

In some embodiments, the parameter analysis module 106 alternatively or additionally uses one or more machine learning models to perform its functionality (e.g., the same model used by the historical heat transfer fluid sample analyzer module 101). For example, the historical heat transfer fluid sample analyzer module 101 may weight and activate a neural node connection via “acceptable,” “marginal,” and “beyond limit” labeled heat transfers fluids based on learning which ranges or other parameter values are associated the most with each of the labels. Responsively, embodiments can determine a distance between a feature vector representing a parameter value (e.g., high molecular weight fraction) of a new heat transfer fluid and a feature vector representing the corresponding parameter value (e.g., high molecular weight fraction). These embodiments are described in more detail below.

In some embodiments, the heat transfer fluid life estimation module 104 and/or the fluid condition estimation module 107 performs its functionality based at least in part on the output performed by the parameter analysis module 106 (or vice versa). In this way, for example, in response to a new heat transfer fluid being registered via the heat transfer fluid registration module 102, this module can programmatically call the parameter analysis module 106, and responsive to this module performing its functionality, it can programmatically call the heat transfer fluid life estimation module 104 (and/or fluid condition estimation module 107).

The fluid condition estimation module 107 is generally responsible for estimating a condition, state, and/or quality of a heat transfer fluid. In some embodiments, such condition or state refers to a score and/or cardinality rating indicating whether the heat transfer fluid is in a “poor/service now/unacceptable,” “fair/service soon,” or “excellent/no service” condition. Quality problems can develop in heat transfer fluids, which will be associated with changes in fluid chemistry and its physical properties when the fluid quality deteriorates (ages) from use or contamination, for example. These can include increased corrosion risk, fouling potential, solids/sludge formation, and pumping difficulties, among others. It is therefore desirable to maintain high quality heat transfer fluid to keep heat transfer fluid systems correctly functioning.

As describe above, in some embodiments, the fluid condition estimation module 107 performs its functionality based at least in part on the parameter analysis module 106. For example, the parameter analysis module 106 may determine whether several parameters of a new heat transfer fluid are within an “acceptable,” “marginal”, or “action” range. In other words, there may be some parameter values tagged to be acceptable, some tagged with “marginal,” and others tagged with “action.” In some embodiments, the fluid condition estimation module 107 aggregates or otherwise uses each of these scores to determine the overall quality of the heat transfer fluid. For example, the fluid condition estimation module 107 may increment or populate a data structure with numerical values to sum up each of these parameter value scores (e.g., acceptable, marginal, and beyond limit). Responsively, the fluid estimation module 107 may compare the numerical values against a rule data set (e.g., conditional logic and imperial knowledge) that specifies threshold and/or weighting information to make its estimations. For example, if a new heat transfer fluid is tagged with “acceptable” ratings over a threshold of X times (or percentage), then the new heat transfer fluid can be tagged overall as being in “excellent” condition. In another example, certain parameters may be more (or less) indicative of a heat transfer fluid quality relative to other heat transfer fluids (e.g., as determined by the historical heat transfer fluid sample analyzer module 101). Accordingly, some embodiments weight various parameters values (e.g., with floating point values or numericals) to offset raw threshold-based calculations based on the importance of specific parameters. Similar functionality can also occur for estimating a fluid life via the heat transfer fluid life estimation module 104. For example, if a particular parameter (e.g., high molecular weight fraction) value determined to be indicative of a particular fluid life is detected in a new heat transfer fluid, then the new heat transfer fluid can be tagged with the particular fluid life prediction.

In some embodiments, the fluid condition estimation module 107 additionally or alternatively performs its functionality based on the functionality performed by the historical heat transfer fluid sample analyzer module 101. For example, using the illustration above, the historical heat transfer fluid sample analyzer module 101 can determine that several heat transfer fluid samples that were determined to be in “fair” condition had a viscosity parameter value between X and Y. Responsively, the historical heat transfer fluid sample analyzer module 101 can tag or flag X and Y as the range of parameter values that are acceptable for a heat transfer fluid being estimated to be in “fair” condition based on analyzing these past historical heat transfer fluid samples. Accordingly, when a new heat transfer fluid is received, if the viscosity parameter value is between X and Y, then the heat transfer fluid may be tagged as being in “fair” condition.

In some embodiments, the fluid condition estimation module 107 uses one or more computer models to perform its functionality. For example, as described above, some embodiments use a machine learning model (e.g., a deep neural network) to weight and activate features or parameters associated with different historical heat transfer fluid samples. For example, various models can establish a ground truth using labels (e.g., a “fair” fluid condition or “acceptable” acidic value number) on various heat transfer fluid samples, each with multiple parameter values. Embodiments can then responsively train on and learn which parameter values are associated with the ground truth. For example, after inputting 30 historical heat transfer fluid samples into a neural network, embodiments can learn that a parameter most indicative of the high quality of a fluid life is a particular molecular weight fraction value. Accordingly, these models can activate the corresponding neural network node connections in order to weight the high molecular weight fraction value parameter or feature such that if a new heat transfer fluid includes this high boiler value, the new heat transfer fluid can be determined to be of “high” quality.

The maintenance recommendation module 108 is generally responsible for generating a recommendation signal to users based at least in part on functionality performed by the heat transfer fluid life estimation module 104, the parameter analysis module 106, and/or the fluid condition estimation module 107. In other words, for example, in some embodiments, in response to the heat transfer fluid life estimation module 104, the parameter analysis module 106, and/or the fluid condition estimation module 107 performing its functionality, one or more of these modules can programmatically call the maintenance recommendation module 108 to perform its functionality.

A “recommendation signal” as described herein refers to a computer-readable instruction, signal, an alert, a natural language message and/or the like that indicates a recommendation of a user or operator to take regarding a heat transfer fluid and/or heat transfer fluid system. For example, in some embodiments, if a heat transfer fluid life estimation, a fluid quality estimation, or parameter score (e.g., acceptable, marginal, or beyond limit), exceeds a threshold, the maintenance recommendation module 108 can transmit instructions to a user device for how to service or treat the heat transfer fluid. In another example, if any of these thresholds are exceeded, the maintenance recommendation module 108 can recommend a change of a heat transfer fluid registered via the heat transfer fluid registration module 102.

In some embodiments, the manner in which the maintenance recommendation module 108 determines which recommendation signals to transmit is based on utilizing a data structure that maps scores and/or thresholds to individual recommendation signals. For example, the maintenance recommendation module 108 can read a lookup table, key-value data store, hash-map, where the key is a fluid quality estimation score or threshold (or heat transfer fluid estimation threshold, or parameter value score), and the value is the specific recommendation signal to make. For instance, if a first parameter is tagged as “beyond limit,” the maintenance recommendation module 108 can read, in a lookup table, what recommendation signal to make when the first parameter is tagged with “beyond limit,” which may be a way to get the values of the first parameter back up to acceptable values.

The historical trend/forecast module 112 is generally responsible for generating historical trend data (e.g., a trend graph) associated with parameter values of heat transfer fluid samples registered via the heat transfer fluid registration module 102 and/or within heat transfer fluid sample data store 105. In some embodiments, the historical trend/forecast module 112 additionally generates forecast predictions (e.g., via exponential smoothing) based on the historical trend data. In an illustrative example of how the historical trend/forecast module 112 may operate, some embodiments determine the “poor” “fair” and “acceptable” scores of parameter values (Y-axis) (described above with respect to the parameter analysis module 106) of a same heat transfer fluid over several years (or other time periods) (X-axis) and responsively determine a trend line or indication (e.g., via a Hough transformation or regression algorithm). The historical trend/forecast module 112 is described in more detail below.

The presentation component 114 is generally responsible for causing presentation of content and related information to user devices, such information determined by the historical heat transfer fluid sample analyzer module 101, the heat transfer fluid registration module 102, the heat transfer fluid life estimation module 104, the parameter analysis module 106, the fluid condition estimation module 107, the maintenance recommendation module 108, and/or the historical trend/forecast module 112. The presentation component may comprise one or more applications or services on a user device, across multiple user devices, or in the cloud. For example, in one embodiment, presentation component 114 manages the presentation of content to a user across multiple user devices associated with that user. Based on content logic, device features, associated logical hubs, inferred logical location of the user, and/or other user data, presentation component may determine on which user device(s) content is presented, as well as the context of the presentation, such as how (or in what format and how much content, which can be dependent on the user device or context) it is presented and/or when it is presented.

In some embodiments, the presentation component 114 generates (or causes generation of) user interface features. Such features can include interface elements (such as graphics buttons, sliders, menus, audio prompts, alerts, alarms, vibrations, pop-up windows, notification-bar or status-bar items, in-app notifications, or other similar features for interfacing with a user), queries, and prompts. In some embodiments, the presentation component 114 generates structured data, tagged data or otherwise causes presentation of structured or tagged data that was previously unstructured, semi-structured, or untagged. For example, in some embodiments the presentation component 108 causes presentation of tagged data (e.g., fluid condition score being “fair” or parameter value being within an “acceptable” range), which may have been previously unstructured or otherwise been in a different form (e.g., existed only in natural language form) than the output provided by the presentation component 114. In this way, some embodiments convert input data to an output that is different than the input.

The consumer application 116 generally refers to one or more computer applications or services, such as online/cloud applications or locally stored apps that consume, include, or utilize some or each of the components of the system 100. In particular, a consumer application 116 may receive an upload of a new heat transfer fluid via the heat transfer fluid registration module 101, after which the various modules 104, 106, 107, 108, and/or 112 performs its functionality.

In some embodiments, the consumer application 116 utilizes the presentation component 114 to cause presentation of related information of related information, which is described in more detail below. Examples of consumer applications 116 may include, without limitation, computer applications (e.g., web applications) or services, such as heat transfer fluid maintenance apps or web applications, social media service applications (e.g., PINTEREST, FACEBOOK, etc.), email, messaging, chat, or any other web application, plugin, extension, or locally stored application.

The historical heat transfer fluid sample data store 105 and/or the registration data store 103 may represent any structured, semi-structured, or unstructured repository to store data, such as a relational database, a data warehouse, a data corpus, image file, and the like. Such data stores can store programming logic, data structures, and the like for performing the functionality, as described herein.

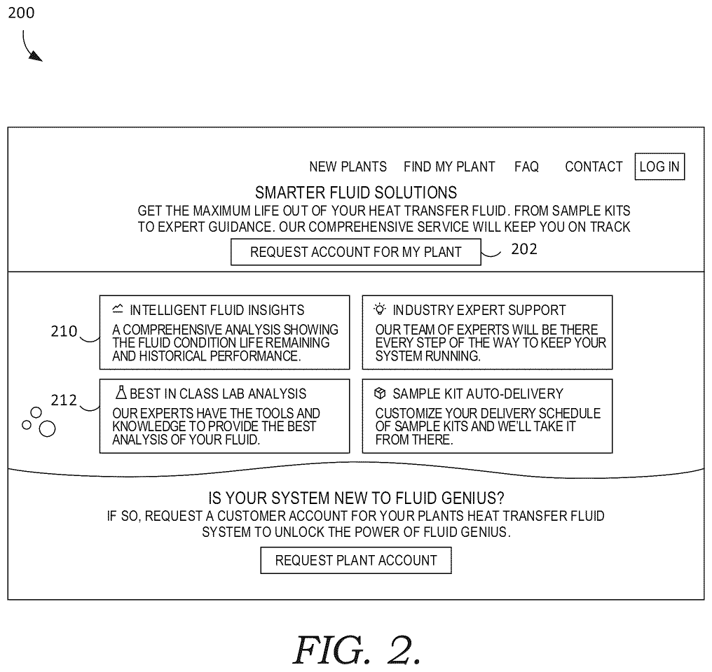

FIG. 2 illustrates a screenshot 200 of an example user interface, according to some embodiments. In some embodiments, the screenshot 200 represents data generated or caused to be displayed by the presentation component 114 of FIG. 1. In some embodiments, the screenshot 200 represents functionality performed by the consumer application 116 of FIG. 1.

In some embodiments, the screenshot 200 represents a landing or home page of a web application or other application. The screenshot 200 represents a page that facilitates user/heat transfer fluid registration or login to perform analyses on a particular heat transfer fluid. In response to receiving an indication that a user has selected the button 202, various embodiments (e.g., the consumer application 116) cause presentation of the fields 204, which are configured to receive users input (i.e., user's first name, last name, and email address). Responsively, in some embodiments, the heat transfer fluid registration module 102 stores this information to the registration data store 103 of FIG. 1. In these instances, users may have never had any heat transfer fluid analyzed before and must therefore register their names and other contact information so that a sample kit can be sent to a user in order to sample one or more of the user's heat transfer fluids to derive various parameter values.

In some embodiments, the sample kit contains components and instructions on how to extract a sample of a heat transfer fluid. Optionally, identification information may be provided along with the sample and/or sample kit that identifies the plant or facility that houses the heat transfer fluid. In some embodiments, the sample kit includes specific sampling components (e.g., sample bottle, sample line, flush pail, fluid drawing mechanism, etc.) and instructions, such as the following. Look over the sample port to make sure it is properly insulated with no leaks and is in good operating order. Prepare the port by starting a cooling water flow through the cooling coils. Open the valve to start the hot fluid flow through the sample cooler. Draw enough fluid into a dry pail to thoroughly clear out the sample line and to verify the fluid is being properly cooled. Draw about 0.5 liter (1 pint) into the sample bottle. Be sure you have a clean container to minimize contamination and increase safety. Secure the sample port valve. Plug and cap the sample bottle promptly and securely. Various types of secondary packaging to capture any fluid may also be present. Place a lid over the flush pail and secure the area. Complete the paperwork and packaging to ship your sample to the fluid supplier where they will analyze the sample in their labs.

In response to receiving an indication that the user has selected the button 208, various embodiments perform functionality related to the tags 210, 202, and 212. In some embodiments, the tag 210 corresponds to functionality as described with respect to the heat transfer fluid life estimation module 104, the fluid condition estimation module 107, and the historical trend/forecast module 112 of FIG. 1. In some embodiments, the tag 212 corresponds to functionality performed by the parameter analysis module 106 of FIG. 1.

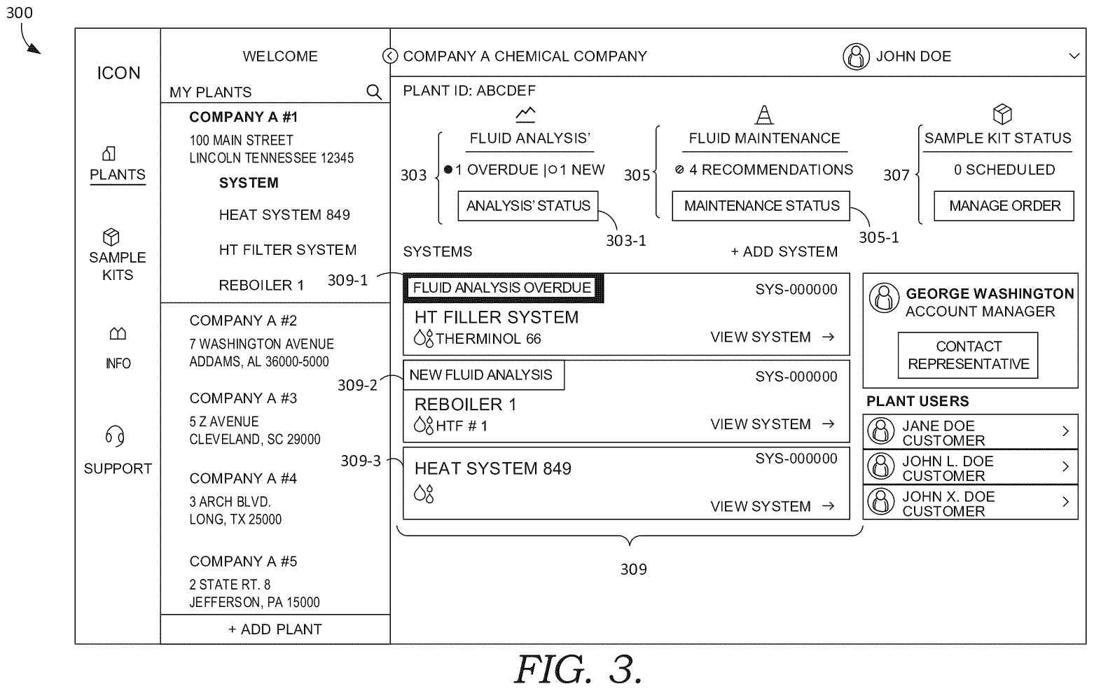

FIG. 3 illustrates a screenshot 300 of an example user interface, according to some embodiments. In some embodiments, the screenshot 300 represents data generated or caused to be displayed by the presentation component 114 of FIG. 1. In some embodiments, the screenshot 300 represents functionality performed by the consumer application 116 of FIG. 1. In some embodiments, the screenshot 300 is caused to be displayed in response to receiving an indication that the button 202 has been selected.

The screenshot 300 illustrates a consolidated view of all fluid analyses performed (or needing to be performed) for all of the user's heat transfer fluid systems. In some embodiments, the fluid analysis indicia 303 corresponds to analyses that the heat transfer fluid life estimation module 104, the parameter analysis module 107, and/or the fluid condition estimation module 107 has performed. In some embodiments, in response to receiving an indication that the user has selected the button 303-1, any one of the estimations or scores generated by the heat transfer fluid life estimation module 104, the parameter analysis module 107, and/or the fluid condition estimation module 107 is caused to be displayed. In some embodiments, the “overdue” indicia corresponds to an indication that heat transfer fluid analysis (e.g., a fluid life or fluid quality estimation) has not been performed yet, but needs to be performed (e.g., because no sample data from a sample kit has been received).

In some embodiments, the fluid maintenance indicia 303 corresponds to analyses that the maintenance recommendation module 108 has performed. In some embodiments, in response to receiving an indication that the user has selected the button 305-1, a recommendation signal, as determined by the maintenance recommendation module 108 is generated. In some embodiments, the sample kit status indicia 307 corresponds to indications of whether or not a user has ordered a sampling kit, or has otherwise set the sampling kit up.

The indicia 309 indicates all of the systems for which heat transfer fluid analysis has been (or needs to be) performed on for a given plant facility. For example, a particular user may have plant that has various machines, each with different purposes and each with different heat transfer fluids that need to be analyzed. Included in the systems are the HT filler system 309-1, the reboiler 1 system 309-2, and the heat system 390-3.

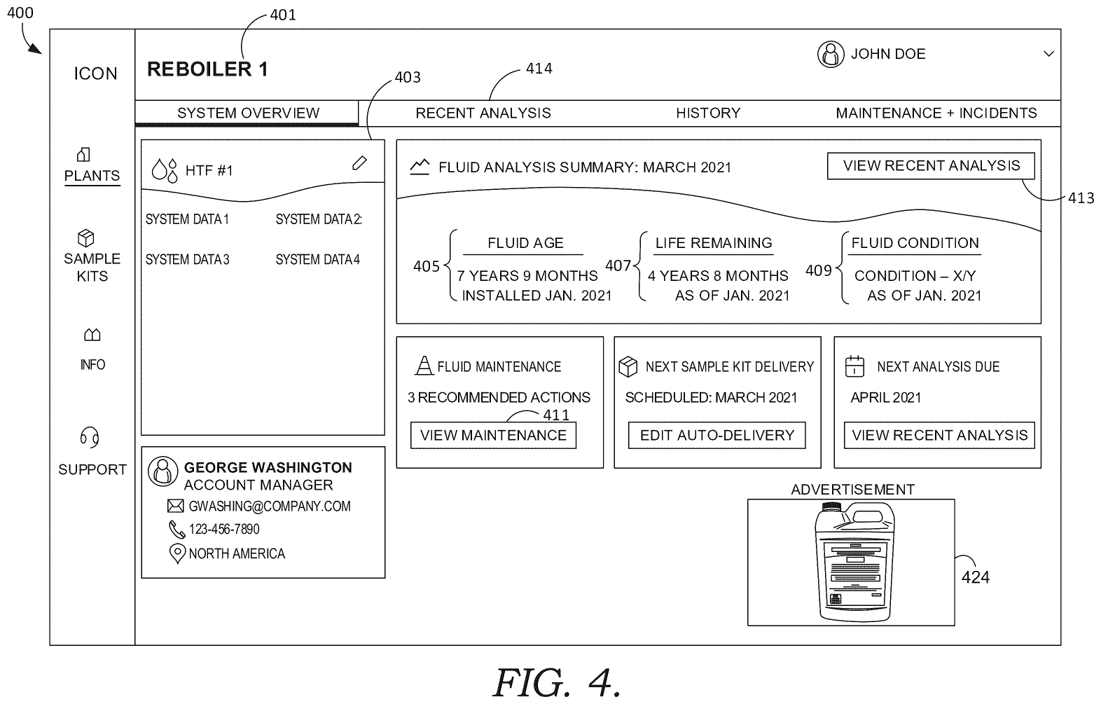

FIG. 4 illustrates a screenshot 400 of an example user interface, according to some embodiments. In some embodiments, the screenshot 400 represents data generated or caused to be displayed by the presentation component 114 of FIG. 1. In some embodiments, the screenshot 400 represents functionality performed by the consumer application 116 of FIG. 1. In some embodiments, the screenshot 400 is caused to be displayed in response to receiving an indication that the user interface element 309-2 of FIG. 3 has been selected.

The screenshot 400 illustrates various attributes associated with a particular heat transfer fluid system, including a fluid analysis summary of heat transfer fluid #1. The particular heat transfer system is the reboiler 1 (e.g., the same system indicated in the user interface element 309-2 of FIG. 3). The user interface element 403 includes various attributes associated with heat transfer fluid #1 (HTF #1), which can be provided by the user at registration time, by a manufacture entity, and/or vendor entity.

In some embodiments, the life remaining indicia 407 (e.g., 1 years and 2 months) represents the output of the functionality performed by the heat transfer fluid life estimation module 104 of FIG. 1 (e.g., a heat transfer fluid life estimation). Likewise, in some embodiments, the fluid condition indicia 409 represents the output of the functionality performed by the fluid condition estimation module 107 (e.g., a heat transfer fluid quality estimation score). In some embodiments, in response to receiving an indication that a user has selected the button 413, various embodiments cause presentation of more or deeper analysis associated with the indicia 407 or 407, as described in more detail below. In some embodiments, in response to receiving an indication that the user has selected the button 411, various embodiments cause presentation of a page or user interface element that indicates the particular recommendation signals determined by the maintenance recommendation module 108 of FIG. 1.

The screenshot 400 of FIG. 4 also includes the banner advertisement 424. A “banner advertisement” is an advertisement that is integrated or embedded within an existing web page (e.g., the screenshot 400) so as to keep the same look and feel of the native web page. For example, the advertisement can include the same format, colors, shape, or the like of an existing web page. This is different than, for example, a pop-up add that includes a flyout or other separate user interface element that is not within the same web page schema. Banner advertisements typically include one or more images (e.g., a digital photograph) or multi-media object (e.g., a GIF or FLASH animation).

Computer network entities, such as network advertisers, typically work with publishers (e.g., a host website) to provide messages (e.g., banner advertisements) that user computing devices receive. For example, a user, browsing the screenshot 400 corresponding to a web application of the publisher, can issue a query using particular key words. A network advertiser corresponding to the advertisement 424 can identify the key words and provide a bid associated with the particular key words to the publisher indicating how much money the network advertiser is willing to pay for an advertisement to be displayed to the user computing device. After network communications between the network advertiser and publisher computing entity are made, the publisher's website (i.e., the screenshot 400) can cause display of a message (i.e., the advertisement 424) on the user computing device. Such a message may describe items for sale, such as a heat transfer fluid, at the network advertiser's electronic marketplace website.

The user may then make or not make various selections or other actions associated with the advertisement 424. In some embodiments, user selections of the advertisement 424 causes the screenshot 400 to change to an advertiser's website via establishing a connection with or opening a communication channel with the advertiser's server(s) so that a user can purchase the heat transfer fluid. Over time, entities can obtain information associated with these selections or actions, such as an estimated conversion rate. A conversion rate is the percentage or proportion of visitors to a website or application that complete some predefined action (e.g., the download of a software instance within the message). The conversion rate can be affected by various factors associated with a message, such as particular message words, message pictures, content of the message, web page message placement, etc. The advertisement 424 can be any suitable message alternative to or in addition to heat transfer fluid. For example, the advertisement 424 can provide local weather-related information, local locations where heat transfer fluid can be purchased, and the like based on automatically extracting a geo-coded indicator (e.g., an IP address or triangulated GPS location) associated with a user device.

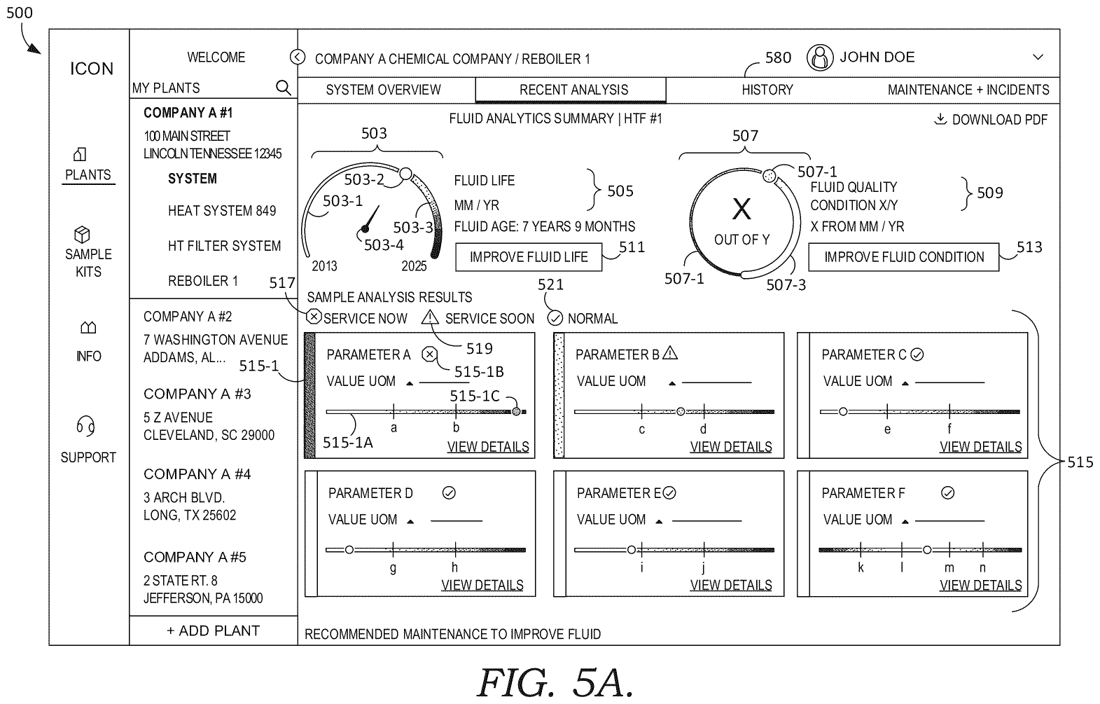

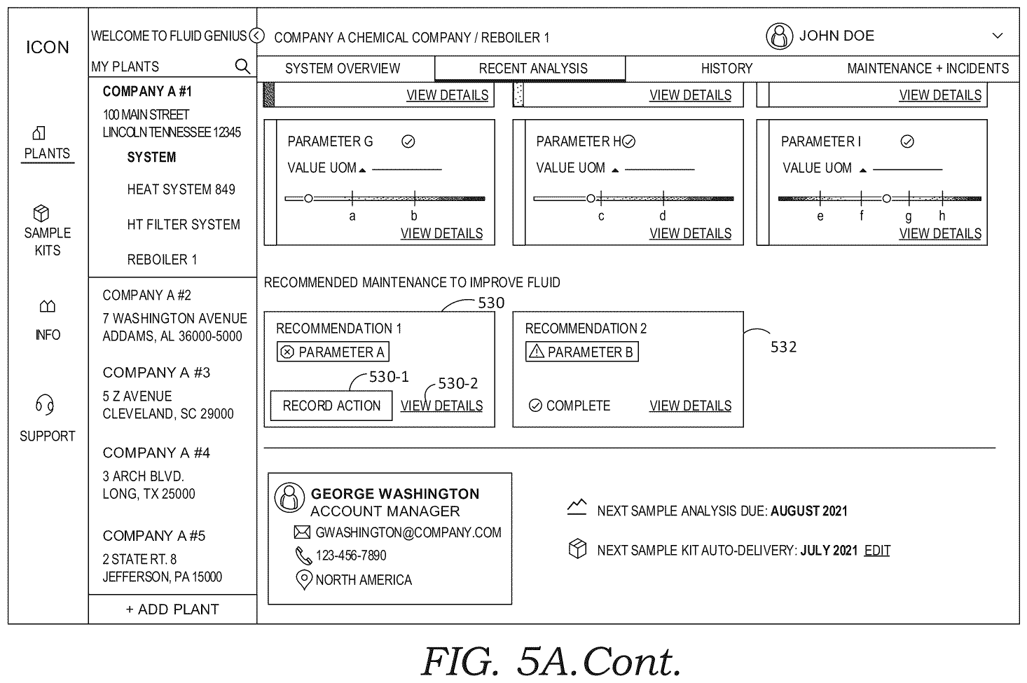

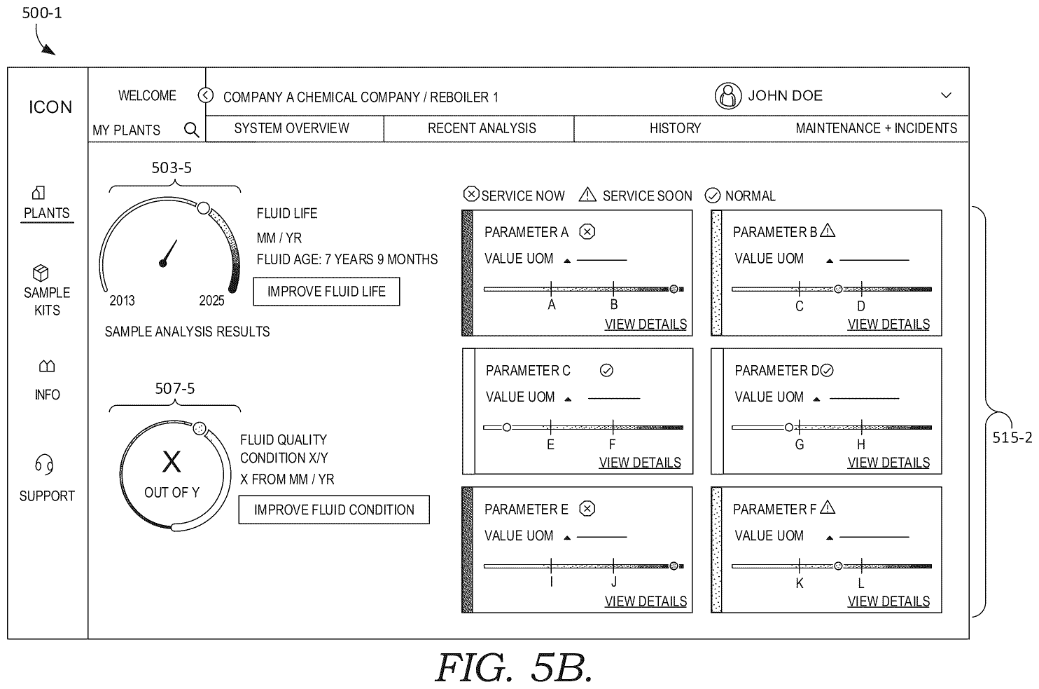

FIG. 5A illustrates a screenshot 500 of an example user interface, according to some embodiments. In some embodiments, the screenshot 500 represents data generated or caused to be displayed by the presentation component 114 of FIG. 1. In some embodiments, the screenshot 500 represents functionality performed by the consumer application 116 of FIG. 1. In some embodiments, the screenshot 500 is caused to be displayed in response to receiving an indication that the button 413 or the tab 414 of FIG. 4 has been selected. For users with multiple sites and systems, the curtain allows for easy comparisons between systems. The dashboard and reports pages will provide an easy means to gain an overview of multiple systems and a general overview.

In some embodiments, the screenshot 500 represents an improved user interface relative to existing user interfaces that indicate various fluid attributes, as described herein. For example, in some embodiments, the screenshot 500 represents a single page (or a home/landing page), where a user can view multiple pieces of information at once in order to avoid drilling down, paging through, scrolling through, or otherwise perform additional user input for better user navigation. For example, instead of the user having to navigate to different pages to view the parameter value information 515, the fluid life remaining estimation 505, and the fluid condition score 509, the user can view all of this information at the screenshot 500 at once. Additionally, the dials 503 and 507, along with various heat map indicators (e.g. within the dials 503, 507, and parameter indicators 515) facilitate quick or less burdensome user analysis, especially, for example, when the user cannot understand the language being presented on the other screenshots.

FIG. 5A illustrates specific a specific fluid life remaining estimation, a fluid condition score, and analysis results of various parameters of a single heat transfer fluid. The dial 503 visually indicates (e.g., via a spindle user interface element 503-4 and heat maps) the fluid life remaining 505. For example, a first portion 503-1 of the dial 503 up through the point 503-2 may be colored green to represent that the heat transfer fluid does not need to be changed or treated (or will not soon need to be changed). However, the portion 503-3 of the dial 503 may transition from yellow (meaning that the heat transfer fluid needs to be changed or treated soon (e.g., within a threshold time period)) to red (meaning that the heat transfer fluid needs to be immediately changed or treated). The spindle UI element 503-4 is pointing to the point 503-2, indicating the current state or fluid life remaining estimation (e.g., which borders a green and yellow color). Other colors may be used as substitutes or in addition to red, yellow and green for those with color blindness, other color preference, or preference for black and white. In this way, users can easily analyze results without undue burden, unlike existing user interfaces.

In some embodiments, the fluid life remaining indicia 505 represents the fluid life estimation performed by the heat transfer fluid life estimation module 104 of FIG. 1. In some embodiments, in response to receiving an indication that the user has selected the button 511, various embodiments cause presentation of the UI element 530, (and/or 532) which indicates that the user can perform system actions to improve the heat transfer fluid life. In some cases, 511 will also show the impact on the score based upon the action taken.

The dial 507 visually indicates (e.g., via the point 507-2 and heat maps) the fluid condition score 509. For example, a first portion 507-1 of the dial 507 up through the point 507-2 may transition from a red color (representing poor heat transfer fluid quality) to a yellow color (representing a fair heat transfer fluid quality) or other logical color progressions. The portion 507-3 of the dial 507 may transition from the yellow color to a green color (representing good or excellent heat transfer fluid quality). The point 507-1, for example, may be located in a yellow region, indicating that the fluid condition score of X (out of Y) is a “fair” or “moderate” score. In this way, users can easily analyze results without undue burden, unlike existing user interfaces.

In some embodiments, the fluid condition score 509 represents the estimation of the quality score performed by the fluid condition estimation module 107 of FIG. 1. In some embodiments, in response to receiving an indication that the user has selected the button 513, various embodiments cause presentation of the UI element 532, (and/or 530) which indicates that the user can take a variety of actions to protect the heat transfer fluid quality.

The sample analysis results indicia 515 indicates several parameter values extracted from a heat transfer fluid sample and whether the corresponding parameter values are determined and tagged as being at an “acceptable” or “normal” 521, or “marginal” 519, or “beyond limit” 517 level. In some embodiments, such determinations are made by the parameter analysis module 106, as described with respect to FIG. 1. The screenshot 500 also indicates individual analysis data for each parameter value. For example, for the UI element 515-1, there is an “beyond limit” indicator 515-1B, as well as a sliding scale UI element 515-1A, which visually indicates, for example, the state of a value, and particularly that is in a poor state, via the point 515-1C (e.g., indicating that some maintenance action (e.g., treating) needs to occur to get the levels back up to acceptable or normal levels. This also improves the user experience because the user can quickly see what the certain levels of parameter values are and whether treating or corrective action needs to occur. The same or similar functionality is performed for all of the other parameter values, as illustrated in the sample analysis results indicia 515.

FIG. 5B is a screenshot 500-1 of an example user interface, according to some embodiments. In some embodiments, the screenshot 500-1 represents the same features and functionality of the screenshot 500 of FIG. 5A, except that particular user interface elements are re-arranged or placed in a different orientation relative to FIG. 5A. For example, the dials 503-5 and 507-5 may represent the same dials and functionality as the dials 503 and 507 of FIG. 5A, but they are located on a left side of the screenshot 500-1. Likewise, the parameter value information 515-5 may represent the same parameter value information 515 of FIG. 5A, except that the information is located on the right side of the screenshot 500-1.

As describe herein, various parameters extracted from heat transfer fluid samples are important or indicative of particular heat transfer fluid condition or life estimates, quality/condition scores, or states when they are at certain levels or ranges (e.g., “acceptable”, “marginal”, or “high”). In some embodiments there are several parameters that that are indictive of heat transfer fluid condition. In other embodiments there are from four to ten fluid condition defining parameters. Provided for convenience below are descriptions of several parameters as they relate to heat transfer fluid condition: viscosity, opacity, moisture content, flash point, insoluble solids, and composition/degradation measures of thermal degradation, acid number, color, base number, pH, flash point, bromine index value, foreign material, and specific gravity. In some embodiments there are 10 parameters used to determine a fluid quality or fluid life. In some embodiments, there are eight parameters used to determine a fluid quality or fluid life, e.g., viscosity, moisture content, flash point, acidity, insoluble solids, composition/degradation measures of thermal degradation, specific gravity and autoignition temperature. In some embodiments, there are six parameters used to determine a fluid quality or fluid life, e.g., viscosity, moisture content, flash point, acidity, insoluble solids, and composition/degradation. In some embodiments, there are four parameters used to determine fluid condition, e.g., viscosity, acidity, insoluble solids and specific gravity. In some embodiments there are 3 or 2 or 1 parameter(s) used to determine fluid condition. These can indicate developing problems, enabling potential corrective actions to protect fluid performance/life.

Viscosity can be readily measured in a fluids lab, e.g., by the ASTM-445 test method, or similar technique. In the ASTM D-445 method, a fluid sample can be held at a precisely controlled temperature while the time for a known volume of the fluid to pass through a calibrated tube is measured. From the elapsed time, the viscosity is calculated.

The fluid's viscosity is a measure of its resistance to flow. Fluids of greater viscosity will require higher-pumping horsepower requirements and will adversely affect the degree of turbulence at heat exchange surfaces which can lower heat transfer coefficients. Not only can elevated viscosity reduce heat transfer performance at high temperatures, it can also affect the ability to pump the heat transfer fluid during cold weather start-up conditions. Viscosity is related to the molecular weight of fluid components. Generally, lower molecular weight components decrease viscosity and higher molecular weight components increase viscosity of the heat transfer fluid. Contamination from leaked process streams, incorrect material added to the heat transfer fluid system, and solvents from system cleaning, as well as thermal stressing and oxidation, may be the source of materials that increase or decrease viscosity. The typical corrective action (e.g., as determined via the maintenance recommendation module 108) to address too low a viscosity would be the removal of low boiling components by circulating the heated fluid through the expansion tank with inert gas purge of the vapor space while venting to a safe location. Condensation and collection for proper disposal of the removed low-boiling organics is recommended unless vented to a properly designed flare. Correcting for high viscosity requires either aged fluid removal and replacement or dilution with unused heat transfer fluid.

Inert gas blanketing is an effective method of minimizing fluid oxidation by blanketing the expansion tank with an inert gas such as nitrogen, carbon dioxide, or natural gas. The purpose of inert gas blanketing is to maintain a nonreactive atmosphere in the vapor space of the expansion tank, preventing the entrance of air and moisture which can adversely affect fluid life. An uninterrupted supply of inert gas, usually nitrogen, controlled by pressure regulators for both inlet and outlet flow may be necessary to obtain this protection. Pressures used should be kept as low as possible inside the expansion tank to minimize inert gas usage. Maintaining a positive pressure slightly over atmospheric barometric pressure is all that may be necessary to prevent air and moisture from entering the tank. A manual vent valve also should be installed to facilitate purging of the expansion tank's vapor space if it becomes necessary

Moisture content may be analyzed by the Karl Fischer titration technique. Moisture content should be kept quite low when operating at high temperatures to avoid issues related to its flashing into water vapor. Inability to maintain low moisture content is an indicator of either an aqueous leak into the system, or perhaps the addition of “wet” make up fluid.

Heat transfer fluid systems and machinery can run heat transfer fluids at differing operating temperatures, which can impact heat transfer fluid life. When operating at very high temperatures, excess moisture content can prevent the ability to circulate the heat transfer fluid due to its flashing into vapor at the circulation pump intake, creating cavitation. Extended operation with cavitation can lead to excessive heat transfer fluid degradation in heater coils due to lower mass flow rates delivered from the pump. Also corrosion may be induced by elevated concentrations of system moisture. In cooling systems, a high moisture content of the fluid will increase the risk of formation of ice crystals on chiller surfaces. This can decrease the efficiency of heat transfer and deteriorate the overall system performance. On new system start-ups, operators may remove residual moisture from the system (from hydrostatic testing) to enable the heat transfer fluid to heat fully to the desired operating temperature. For systems in operation, increasing moisture content may be caused by in-leakage of water from aqueous process steams or from steam systems, or by moisture intake via an expansion tank open to atmosphere. Excess moisture can typically be vented from the expansion tank using the low-boiler venting method. To achieve low ppm moisture levels required for the cooling operation, molecular sieves can be placed in side stream operation.

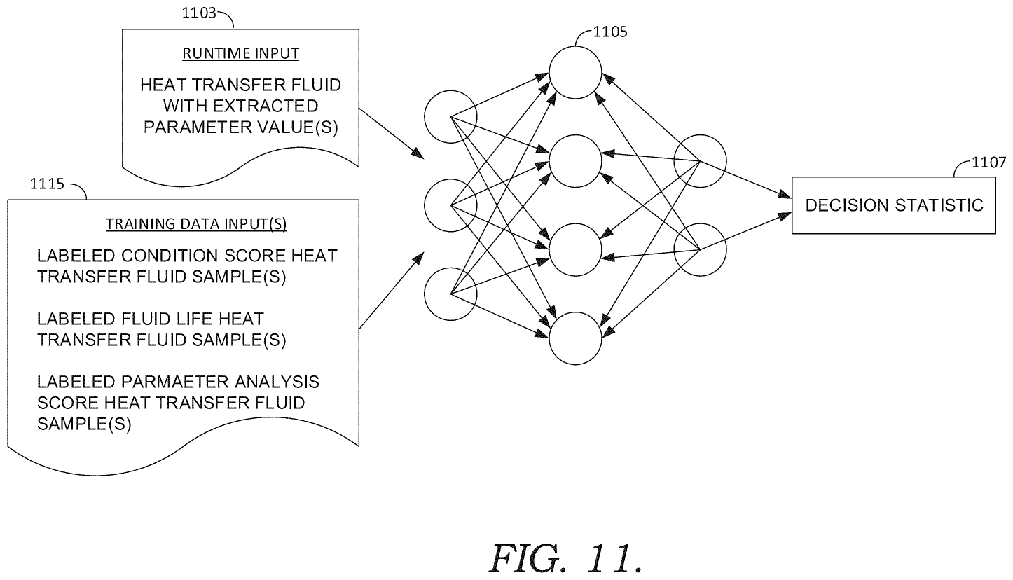

Because particular high operating temperatures can affect heat transfer fluid, such as moisture content, this can negatively impact the fluid life expectancy score. As described above, cavitation as well as corrosion (which results from excessive moisture) can lead to excessive heat transfer fluid degradation in heater coils due to lower mass flow rates delivered from the pump. In some embodiments, one or more machine learning models (e.g., the neural network 1105 of FIG. 11) learns a mapping for specific operating temperatures (as well as other parameters, such as high boilers and on-stream times) and corresponding life expectancy scores. For example, training data may indicate that for a first set of heat transfer fluid samples of type A where the operating temperature was B (or within a range threshold of A), the actual fluid life was C (or within a range threshold of C). The training data may further indicate that for a second set of heat transfer fluid samples of type A, where the operating temperature was D (or within a range threshold of D), the actual fluid life was E (or within a range threshold of E). Accordingly, for a currently analyzed heat transfer fluid type A, if the operating temperature was B, the predicted fluid life may be C (instead of E) based on the historical heat transfer fluid samples' fluid life readings for the same operating temperature with respect to the same heat transfer fluid type.

Alternatively or additionally, one or more programmatic rules or policies may be implemented to map, via one or more data structures, the specific operating temperatures (as well as other parameters, such as high boilers and on stream times) and life expectancy scores. For example, a lookup table or hash map can be created where there the first column includes the operating temperature values used as a key to look up corresponding life expectancy scores. For example, a first record can include a key that indicates temperature values between A and C, as well as a corresponding value X, which indicates a specific life expectancy scores. A second record can include another key that indicates temperature values between D and F, as well as corresponding value Y, which indicates another life expectancy score. Accordingly, for example, if a currently analyzed heat transfer fluid has an operating temperature B, the first record can be queried (because the operating temperature is between A and C), and the corresponding fluid life expectancy score can be generated. In some embodiments, such data structures are generated where the fluid life estimation is inversely proportional to the operating temperature. That is, the higher the operating temperature, the lower the fluid life expectancy score and the lower the operating temperature, the higher the fluid life expectancy score. Similar helper lookup tables can be used for other parameters described herein to estimate the total fluid life, such as tables for high boiler percentage and on-stream time.

The flash point of a high-temperature fluid is commonly measured by the Cleveland Open Cup (COC) method, ASTM D-92. A closed cup technique is also useful in classifying fluids and is run per ASTM D-93. The flash point is the lowest temperature of the fluid under the test conditions where ignition of the vapors above the liquid can occur, yet evaporation rate is too low to sustain combustion. Flash points are important in electrical classification and hazard analysis.

While many heat transfer fluids have relatively high flash points, they often are not classified as fire resistant. However, heat transfer fluid systems are typically closed systems. Therefore, a release of fluid may only occur in case of accidents or malfunctions and it is typically safe to operate such well-designed and maintained systems and fluids even at temperatures well above the fluid's flash point. Flash point is a property to be considered in the hazard evaluation of operating systems with combustible fluids. A significantly depressed flash point of the in-service heat transfer fluid may not only increase the fire hazard in case of leakages and the presence of an effective ignition source, it may also affect the area electrical classification of the system in extreme cases. Typically, routine venting of low-boiling thermal degradation products from the expansion tank to a safe location will maintain the fluid's open-cup flash point to within 25° C. (45° F.) of the flash point of unused fluid.

Acidity of fluids is commonly measured by ASTM D-664, which is a potentiometric titration. Fluid oxidation results in accumulations of carboxylic acids which lower the apparent pH or raise the total acid number (TAN). Typically, unused organic heat transfer fluids will have a near zero acid number.

High acid numbers could indicate severe fluid oxidation, which is most often a result of hot fluid exposure to air in the expansion tank. But they may also indicate possible contamination from improper material added to the heat transfer fluid system inadvertently or fluid leaked from the process side of heat exchangers. If the acidity becomes excessive, the machine components could corrode and fail. Oxidation and corrosion products can form sludge and deposits that can also decrease heat transfer rates by fouling. A condition of this nature is typically best corrected by removing the material and replacing it with new fluid, with serious consideration given to a system flush to remove residual acidity. If the high acidity was caused by oxidation, inerting the vapor space in the expansion tank should be considered. System inerting is a highly effective means of protecting against unwanted increases in fluid acidity and oxidative degradation.

Insoluble solids content is essentially a measure of the concentration of solids in the fluid at room temperature. Organic solids result from exceeding their solubility limit in the fluid. Other solids can include carbon, small portions of gasket materials and metal shavings, and some rust.

The presence of solvent (e.g., acetone or pentane) insoluble solids generally indicates contamination from dirt, corrosion products, or severe oxidative or thermal stressing. This condition may cause fouling of heat transfer surfaces which would deteriorate heat transfer performance. Also, plugging of small diameter lines or narrow heat transfer passages could occur. Finally, large amounts of insoluble solids may contribute to wear and plugging of mechanical seals and valves resulting in equipment failure, operational problems, and increased maintenance requirements. If these problems occur, side stream filtration can usually provide ongoing protection against solids-related deposits and their potential consequences. If solids contamination is extremely high, fluid may need to be removed for external filtration and the system may need to be cleaned. Specialized flushing fluids, designed by heat transfer fluid suppliers, can be effective in removing fouling deposits from most synthetic and mineral oil fluid systems. Modest solids content may require filtering with successively finer rated filter element sizes to get the situation under control. A suggested filter rating generally is 10 to 25 microns for ongoing fluid maintenance.

Gas chromatography allows the quantification of compounds which have boiling points lower than the initial boiling point (low boilers [LBs]) and higher than the final boiling point (high boilers [HBs]) of the unstressed heat transfer fluid. This analysis provides a measure of the degree of fluid degradation experienced and can provide an indicator of organic contamination. Gas chromatography typically does not directly provide a measure of inorganic contamination of organic fluids.

Thermal cracking of the heat transfer fluid will result in components which are lower in molecular weight and commonly are known as low boilers (low molecular weight fraction). High boilers (high molecular weight fraction) also can be generated when some compounds recombine to produce higher molecular weight materials. Both low- and high-boiling degradation products can create an unfavorable environment for efficient heat transfer system operation.

Low-boiling components can affect system operation in several ways. First, when present in significant quantities, low boilers can lead to pump cavitation. Severe cases may cause damage to pump seals and, if allowed to continue uncorrected, can damage impellers. Second, when low boilers are present in excessive concentrations, the heat transfer fluid flashpoint and viscosity may be lowered. Third, the increased fluid vapor pressure resulting from the presence of low-boiling components can cause premature and unexpected pressure relief and venting. Finally, excessively rapid formation of low boilers will result in unacceptably high fluid make up costs as the low boilers removed from the system are replaced with fresh fluid. Removal of low boilers is typically accomplished by venting from the expansion tank to a safe location.

Since an expansion tank is usually installed at a high point in the system, it also can serve as the main venting point of the system for excess levels of low boilers and moisture which may accumulate in the heat transfer fluid. To properly vent a heat transfer fluid system, the expansion tank should be capable of accommodating the circulating flow of hot heat transfer fluid. To remove low boilers, the temperature in the expansion tank should be increased and the tank pressure may be lowered while venting. As they flash into the vapor space, the excess low boilers and moisture can be more effectively removed by sweeping out the expansion tank through the vent line to a safe area (preferably via a cooled condenser). Modest pressure decreases help minimize the loss of good heat transfer fluid in the vent stream.

The presence of high boilers can increase heat transfer fluid viscosity, which will affect the fluid's pump-ability at low temperatures and the system's heat transfer efficiency. Unlike low boilers, high-boiling compounds are typically not removed from the system easily once they are formed. Hence, high boilers continue to accumulate until the maximum recommended concentrations are reached, until the end of the fluid life. If high-boiler concentrations are allowed to accumulate beyond that point, sludge and tar deposits can form as the solubility limits for the higher molecular weight compounds are exceeded. Added costs of operation as a result of these sludge deposits include downtime, repairs, clean-out, and lost production. Corrective action would be either a replacement of the fluid or a major dilution with virgin fluid to maintain fluid properties within acceptable range.

Various embodiments of the present disclosure cause these corrective actions described above (or other physical actions) to occur based at least in part on a heat transfer fluid life estimation or fluid condition (quality) score exceeding or falling outside of some threshold. For example, various embodiments of the present disclosure can send a control signal (e.g., a recommendation signal) to a heat transfer fluid system, which automatically causes some notice that a corrective action is needed, such as removing a panel on a heat transfer fluid system machine or make other physical or mechanical or chemical interventions

In another example, various embodiments cause delivery (e.g., automatically or upon user request) of a new heat transfer fluid to a particular plant (e.g., when it has been detected that a heat transfer fluid life of an existing heat transfer fluid will end soon). In another example, some embodiments cause a heat transfer fluid to be changed (or treated) in a heat transfer fluid system (e.g., via user install or automatically via a control signal). In yet another example, a control signal can be transmitted to a heat transfer fluid system (or user device), which emits an auditory or visual indicator indicative of corrective action needing to be taken based on the fluid life estimation or fluid quality estimation score. In yet another example, various embodiments cause a cooling or heating of a heat transfer fluid (whichever is required by the heat transfer fluid system) to obtain a particular temperature based on the fluid life estimation and/or fluid condition score. For example, based on the fluid life not exceeding a fluid life estimation threshold (e.g., the fluid life has not expired) or condition score threshold (e.g., the heat transfer fluid is of high quality), the heat transfer fluid system can continue to function for heating or cooling. Conversely, if any of these thresholds have been met, then the heat transfer fluid system can be treated/changed (e.g., perform system venting). Responsively, the heat transfer fluid system can be activated to continue its function as heating or cooling.

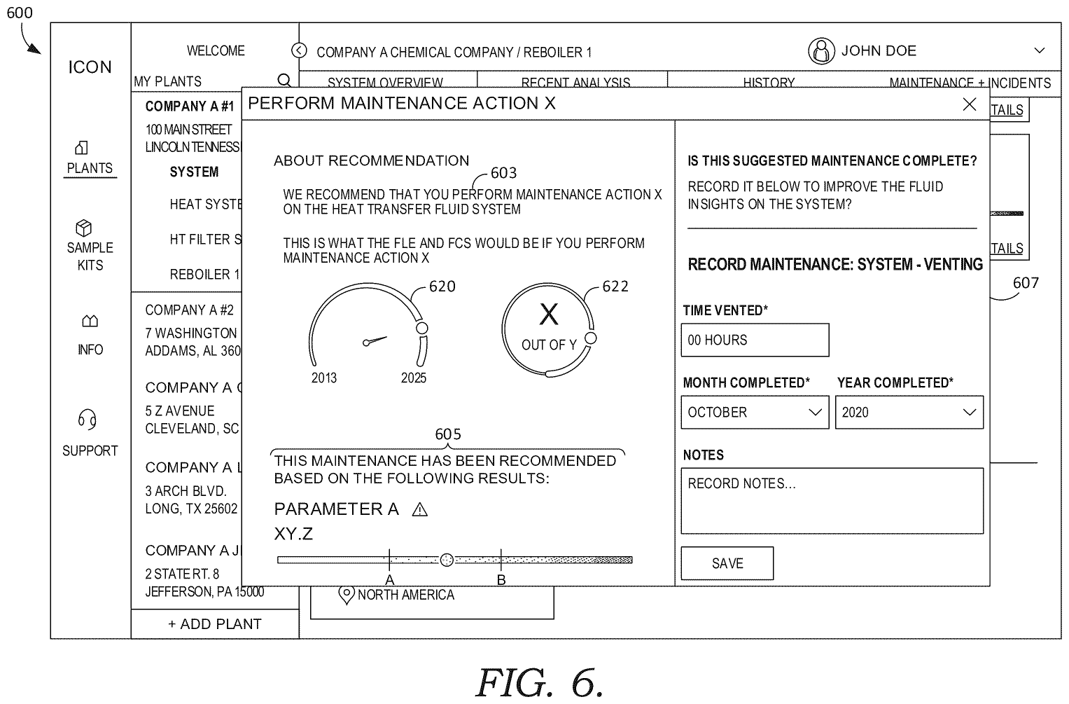

Returning now to the figures, FIG. 6 illustrates a screenshot 600 of an example user interface, according to some embodiments. In some embodiments, the screenshot 600 represents data generated or caused to be displayed by the presentation component 114 of FIG. 1. In some embodiments, the screenshot 600 represents functionality performed by the consumer application 116 of FIG. 1. In some embodiments, the screenshot 600 is caused to be displayed in response to receiving an indication that the link 530-1 of FIG. 5A has been selected. In some embodiments, the information indicated in the screenshot 600 is determined by the maintenance recommendation module 108, as described with respect to FIG. 1 Some features may include the ability to hover over various data points to gain further information on those points. Additionally, some of the graphics may be expandable for better viewing. The graphics have been designed for ease of copy/pasting into common software such as MS Office.

The screenshot 600 illustrates detailed information for recommended maintenance to improve a heat transfer fluid. The UI element 603 illustrates specific information about maintenance action X (e.g., system venting) that should be taken in order to service the corresponding heat transfer fluid. The UI element 605 indicates (similar or identical to the sliding scale 515-1C or 515-1) the parameter and its associated value—parameter A—responsible for the maintenance recommendation. The UI element 607 is configured to receive and cause storage, in computer memory, of user input that indicates when the user completed the corresponding system venting (and/or other maintenance recommendation), as well as additional notes. Some embodiments use Natural Language processing (NLP) on natural language sequences entered in the “notes” field in order to tokenize/parse the sequences, tag them with POS (e.g., noun, adverb, adjective) or entity identifiers (e.g., product type, heat transfer fluid type, etc.) so that semantic meaning (as opposed to syntactic) and relationships can be determined. For example, some embodiments process the natural language characters to modify a fluid quality score or fluid life score (e.g., based on the user stating that they have treated the fluid in some specific way).

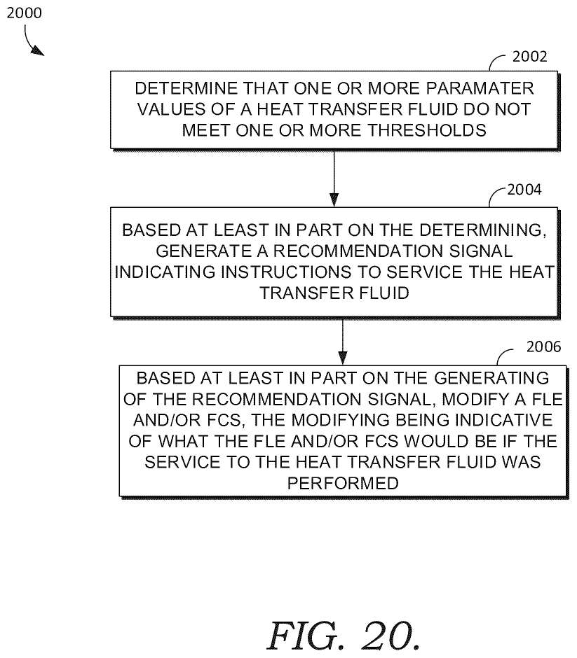

FIG. 6 further includes indicator 602, which indicates what the estimation of life remaining (i.e., a FLE) of the heat transfer fluid would be if the user performs maintenance action X. FIG. 6 further includes indicator 622, which indicates what condition (i.e., a FCS) the heat transfer fluid would be in if the user performs maintenance action X. In some embodiments, the calculation or modification of the FLE and/or FCS as indicated in 620 and 622 are performed as described with respect to the process 2000 of FIG. 20.

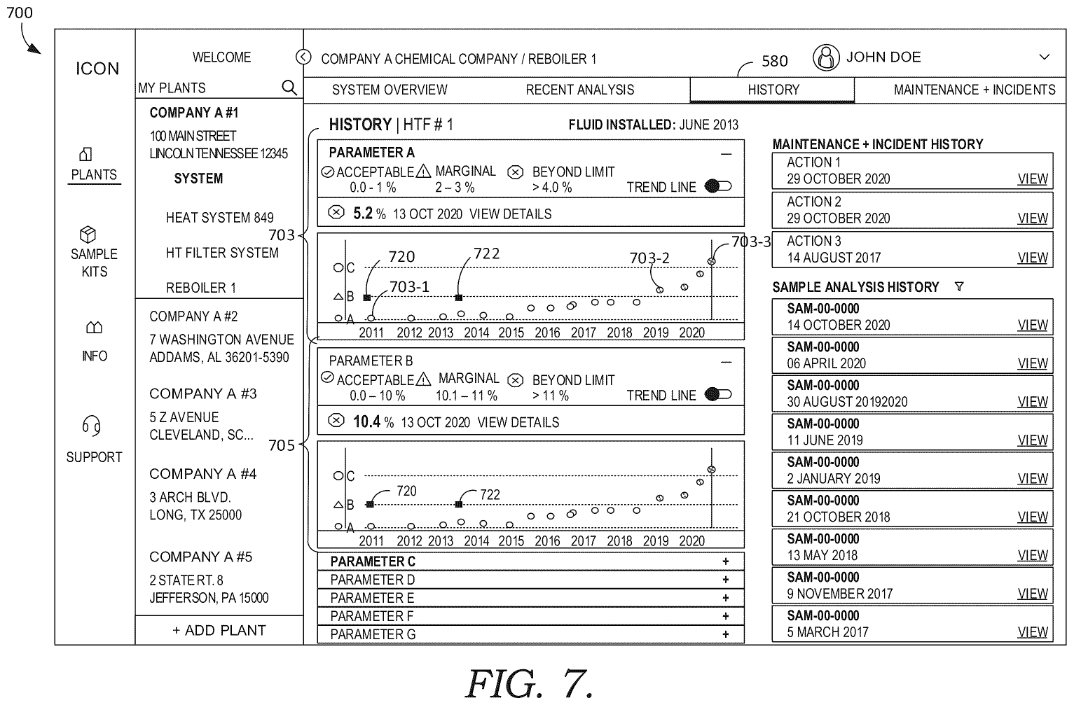

FIG. 7 illustrates a screenshot 700 of an example user interface, according to some embodiments. In some embodiments, the screenshot 700 represents data generated or caused to be displayed by the presentation component 114 of FIG. 1. In some embodiments, the screenshot 700 represents functionality performed by the consumer application 116 of FIG. 1. In some embodiments, the screenshot 700 is caused to be displayed in response to receiving an indication that the tab 580 (also indicated in FIG. 5A) has been selected. In some embodiments, the information indicated in the screenshot 700 is determined by the historical trend/forecast module 112, as described with respect to FIG. 1 This module may also remove outliers, remove designate data points or series of data points based on data and/or user needs. In some cases this data may be exportable to other user data systems.

The screenshot 700 illustrates the levels or limits acceptable or marginal for individual parameters of a heat transfer fluid over a time period (from 2011 to 2020), which shows historical trends of the ratings over time. The Y-axis of the graphs indicate whether the parameter value levels were “acceptable,” “marginal,” or “beyond limit” and the X-axis of the graphs indicate the year. In other words, the same heat transfer fluid #1 may have been sampled at various points in time to derive parameter value information for the same parameters. For example, the UI element 703 indicates from 2011 to around 2019, the parameter A value was at “acceptable” levels. For instance, in some embodiments, each point, starting at point 703-1 to point 703-2 may show a green color indicating an “acceptable” score. The UI element 703 also indicates from around 2011 to 2020, the Parameter A value was at a “marginal” level (e.g., the points between 703-2 and 703-3 are yellow colors). The UI element 703 also indicates that just past 2020, the Parameter A value was at a “beyond limit level” (e.g., the point 703-3 is a red color). The same or similar analysis can be performed for other parameters, such as Parameter B, as indicated in the UI element 705.

Some embodiments cause a trend line or curve (not shown) to be drawn through the points (e.g., from point 703-1 to 703-3) (e.g., via regression or other techniques, such as the Hough transformation algorithm). Some embodiments only cause display of such line or curve in response to the data showing an appropriate confidence level (or some other threshold) that the trend is valid and/or based on other rules (e.g., when a parameter is currently at an “beyond limit” level or the overall fluid quality score fluid life estimation is below some threshold). The confidence level or thresholds can be any suitable value or ranges, such as less than 85%, 85-100 percent, 90-100 percent, and the like.

FIG. 7 further includes additional indicators 720 and 722 within the graphs 703 and 705, which indicate the point in time at which particular maintenance actions were taken. Such indicators gives users a visual sense not only of when certain maintenance actions were taken but how such maintenance actions affect fluid life expectancy or fluid condition scores. For example, based on circulating heated fluid through an expansion tank with inert gas purge of the vapor space while venting to a safe location in conformance with a recommendation in the year 2011 (corresponding to indicator 720), the parameter A and B levels may drop or otherwise be maintained within acceptable or marginal levels as illustrated in 703. Accordingly, the user may easily be able to determine that at least one reason for the satisfactory readings between 2011 and 2013 may be because of the particular removal of boiling components maintenance action by viewing or otherwise engaging with the indicator 720.

In some embodiments, in response to receiving an indication of user engagement of the indicators 720 or 722, additional metadata associated with the given maintenance action is caused to be presented. For example, in response to receiving an indication that a cursor has hovered over the indicator 720, particular embodiments automatically cause presentation of a pop-up window that includes various corresponding information, such as the specific maintenance action taken, a description of how the maintenance action will help heat transfer fluid life or condition, the specific date and time of the maintenance action taken, how often the maintenance action should occur, or the like. In some embodiments, such information is additionally or alternatively provided in response to receiving an indication that a user has selected the “view” link 730.

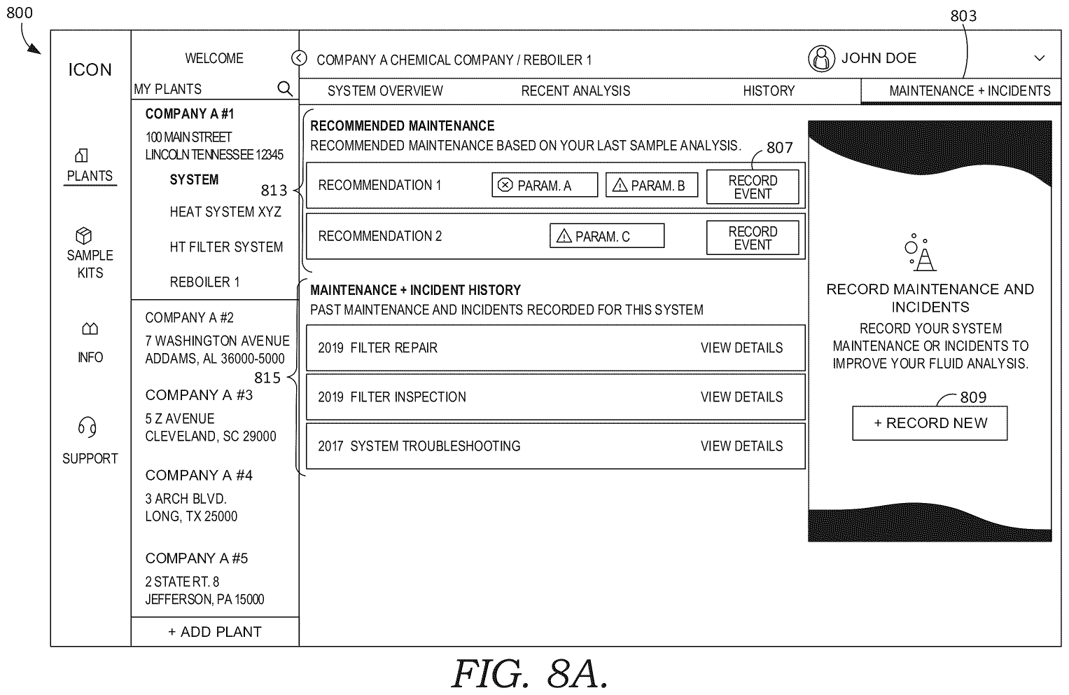

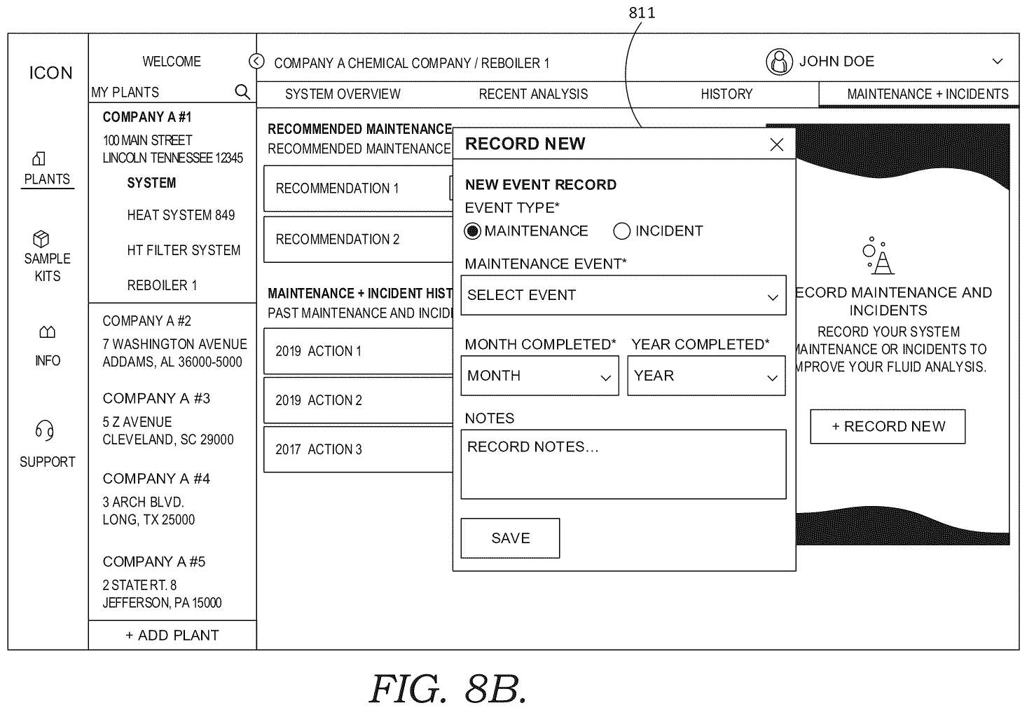

FIG. 8A illustrates a screenshot 800 of an example user interface, according to some embodiments. In some embodiments, the screenshot 800 represents data generated or caused to be displayed by the presentation component 114 of FIG. 1. In some embodiments, the screenshot 700 represents functionality performed by the consumer application 116 of FIG. 1. In some embodiments, the screenshot 800 is caused to be displayed in response to receiving an indication that the tab 803 or button 411 of FIG. 4 has been selected. In some embodiments, the information indicated in the screenshot 800 is determined by the maintenance recommendation module 108, as described with respect to FIG. 1.

FIG. 8A indicates a maintenance history associated with a sample analysis of various parameters, according to various embodiments. As described above, in some embodiments (e.g., FIG. 6), based on certain parameter values falling within an “beyond limit” range or “marginal” range, certain recommendation signals may be made, such as performing specific recommendations, as illustrated by the indicia 813 of FIG. 8A. The indicia 815 indicates a history of each corrective action or other maintenance event (e.g., filter repair) taken for a given heat transfer fluid or heat transfer fluid system.

In some embodiments, in response to receiving an indication that the user has selected the button 807 or 809, the window 811 of FIG. 8B is caused to be presented. The window 811 includes various fields that are configured to receive user input regarding a particular corrective or maintenance even taken, such as the name of the maintenance event, the month the maintenance event was completed, the year the maintenance event was completed, and notes. In some embodiments, NLP is used to perform computer processing on any natural language characters input into the “notes” section, as described above with respect to the element 607 of FIG. 6.

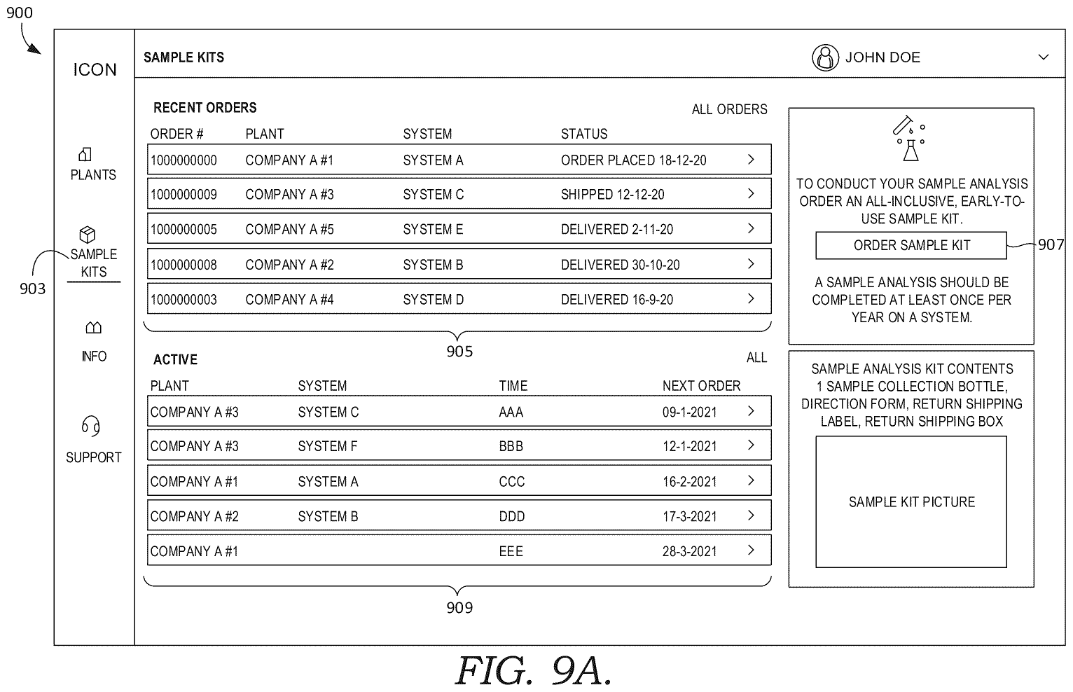

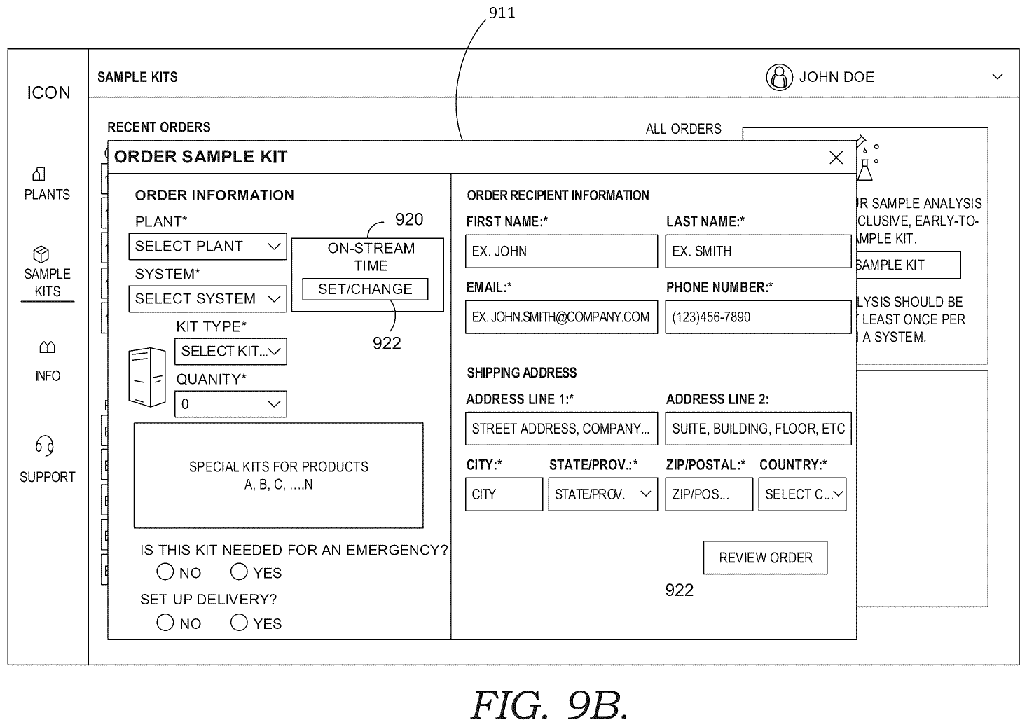

FIG. 9A illustrates a screenshot 900 of an example user interface, according to some embodiments. In some embodiments, the screenshot 900 represents data generated or caused to be displayed by the presentation component 114 of FIG. 1. In some embodiments, the screenshot 700 represents functionality performed by the consumer application 116 of FIG. 1. In some embodiments, the screenshot 900 is caused to be displayed in response to receiving an indication that the UI element 903. has been selected. In some embodiments, the information indicated in the screenshot 900 is determined by the heat transfer fluid registration module 102, as described with respect to FIG. 1.

FIG. 9A indicates recent orders and automatic shipments of one or more sample kits a customer has made. As described herein, a user can order a sample kit (also referred to as a “sample analysis kit”) in order to extract parameter values (e.g., the parameter values indicated in the UI element 515 of FIG. 5A) for a particular heat transfer fluid. The UI element 905 indicates a record of each order of a sample kit for a given plant, heat transfer fluid system, and the shipment status of the order. The UI element 909 indicates automatic shipments of sample kits for a given heat transfer fluid system, the time at which the sample kits are shipped or ordered, and the next shipment of the sample kits. In some embodiments, in response to receiving an indication that the user has selected the button 907, the window 911 of FIG. 9B is caused to be presented. The window 911 includes several fields that are configured to receive user input so that a sampling kit can be sent to a user, such as a plant ID, heat transfer fluid ID, first name, last name, email address, phone number, shipping address, and the like.

FIG. 9B also includes the on-stream time window 920, which includes the button 922. “On-stream time” refers to the quantity of time (or percentage of time) a user's particular heat transfer fluid is to be used in heat transfer or otherwise operating in a corresponding heat transfer fluid system. For example, the on-stream time may refer to the amount of hours in a day that a heat transfer fluid system is to perform heat transfer via the heat transfer fluid.

The on-stream time can impact a fluid life expectancy score or fluid condition score in an inversely proportional manner. Generally, the more time a heat transfer fluid is in use, the lower the fluid life expectancy score or fluid condition score and the less time a heat transfer fluid is in use, the higher the fluid life expectancy score. In some embodiments, one or more machine learning models (e.g., the neural network 1105 of FIG. 11) learns a mapping for specific on-stream times and corresponding life expectancy scores or fluid condition scores. For example, training data may indicate that for a first set of heat transfer fluid samples of type A where the on-stream time was B hours (or within a range threshold of B), the actual fluid life was C (or within a range threshold of C). The training data may further indicate that for a second set of heat transfer fluid samples of type A, where the on-stream time was D hours (or within a range threshold of D), the actual fluid life was E (or within a range threshold of E). Accordingly, for a currently analyzed heat transfer fluid type A, if the on-stream time was B hours, the predicted fluid life may be C (instead of E) based on the historical heat transfer fluid samples' fluid life readings for the same on-stream time with respect to the same heat transfer fluid type.

Alternatively or additionally, one or more programmatic rules or policies may be implemented to map the specific on-stream times (as well as other parameters, such as a heat transfer fluid's bulk operating temperature and high boiler percentage) to corresponding life expectancy scores (or fluid condition scores). For example, if on-stream time was A, the operating temperature was B and the high boiler percentage was C, then the fluid life may be predicted to be D, whereas if the on-stream time was E (with the other parameter values being constant), there may be a slight predicted change in the fluid life.

The button 922 allows users to set their fluid on-stream time in accordance with their current output of on-stream time of heat transfer fluids within their systems. Accordingly, in response to receiving an indication that the user has selected the button 922, particular embodiments can cause presentation of a field or other user interface element (e.g., a predefined selectable range) that allows a user to input the on-stream time. In response to receiving an indication that such input has been provided, particular embodiments update or otherwise set a fluid life expectancy or fluid condition score (e.g., 407 or 409).

In some embodiments, the on-stream time window 920 can be generated at any user interface screenshot described herein. In this way, users can set on-stream time alternative or in addition to a time that a user is registering a heat transfer fluid sample. For example, after a user has already sent in a heat transfer fluid via a kit, as described with respect to FIG. 9B, the user can change or adjust the on-stream time via the window 920 within the screenshot 400 of FIG. 4. This accounts for embodiments where on-stream times can periodically change.

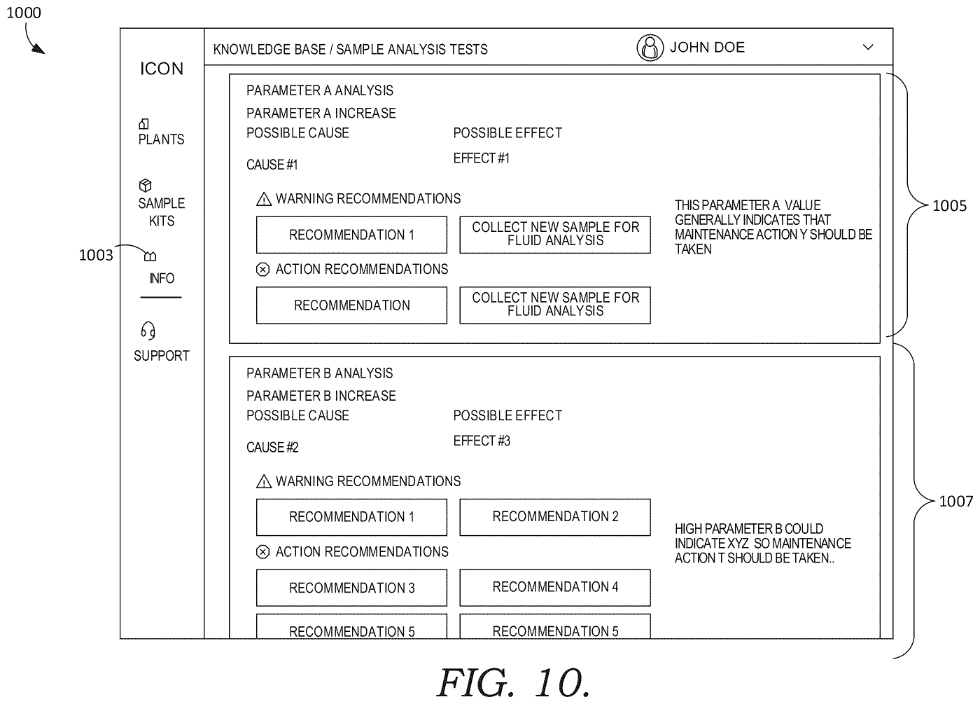

FIG. 10 illustrates a screenshot 1000 of an example user interface, according to some embodiments. In some embodiments, the screenshot 1000 represents data generated or caused to be displayed by the presentation component 114 of FIG. 1. In some embodiments, the screenshot 1000 represents functionality performed by the consumer application 116 of FIG. 1. In some embodiments, the screenshot 1000 is caused to be displayed in response to receiving an indication that the UI element 1003 has been selected. In some embodiments, the information indicated in the screenshot 1000 is determined by the maintenance recommendation module 108, as described with respect to FIG. 1.

The screenshot 1000 illustrates information about several parameters and various recommendations (e.g. corrective actions) that can be taken based on parameter analysis. For example, the UI element 1005 indicates parameter A information, UI element 1007 indicates parameter B information. The UI element 1005, for instance, indicates that when the alert is a “marginal” alert for the parameter A, the operator should perform system maintenance and/or collect a new sample for fluid analysis. When the alert is a “beyond limit” alert, the user should perform maintenance action Y and collect a new sample for fluid analysis (e.g., after the maintenance action Y). In some embodiments, the screenshot 1000 includes some or all of the parameter information described with respect to FIG. 5A (e.g., information about each parameter and each corrective action that can be taken).

FIG. 11 is a schematic diagram illustrating how a decision statistic is generated using one or more machine learning models, according to some embodiments. In some embodiments, FIG. 11 represents the data and functionality used by the heat transfer fluid life estimation module 104, the parameter analysis module 106, and/or the fluid condition estimation module 107 of FIG. 1.

FIG. 11 illustrates that the runtime input 1103 is fed or processed through the one machine learning model(s) 1105 to make a prediction, such as the particular quality or condition score of the heat transfer fluid, the fluid life score, and/or the parameter analysis score. Such input 1103 includes a heat transfer fluid, as well as several heat transfer fluid parameter values that have been extracted (e.g., measured after receiving a heat transfer fluid sample via a sample kit).

The one or more machine learning models 1105 generates one or more particular decision statistic predictions 107 (e.g., a classification prediction of a classifier model, a clustering prediction of a clustering model, or a regression prediction for a regression model) given the runtime input 1103 and the training data input(s) 1115. Such machine learning model(s) 1105 may be any suitable model of any suitable type. For example, such machine learning model(s) 1105 can be supervised or unsupervised and can be or include a neural network (e.g., a Convolutional Neural Network (CNN) or Siamese Neural Network), decision trees, random forests, support vector machine, Naïve Bayes, and or clustering (e.g., K-means clustering). Accordingly, although the machine learning model(s) 1105 is represented as a neural network, it is understood that any suitable machine learning model (or combination of models) can alternatively or additionally be used. In an illustrative example of the decision statistic(s) 1107, the machine learning model(s) 1105 may cluster or classify a feature vector representing some or all of the input(s) 1103 in a cluster or group representing “fair” heat transfer fluids.

In certain embodiments, the decision statistic(s) 1107 may either be hard (e.g., membership of a class is a binary “yes” or “no”) or soft (e.g., there is a probability or likelihood attached to the labels). Alternatively or additionally, transfer learning may occur. Transfer learning is the concept of re-utilizing a pre-trained model for a new related problem.

In some embodiments, the machine learning model(s) 1105 converts or encodes the runtime input 1103 and/or training data input(s) 1115 into corresponding feature vectors in feature space. A “feature vector” (also referred to as a “vector”) as described herein includes one or more real numbers, such as a series of floating values or numerical values (e.g., [0, 1, 0, 0]) that represent one or more other real numbers, a natural language (e.g., English) word and/or other character sequence (e.g., a symbol (e.g., @, !, #), a phrase, and/or sentence, etc.). Such natural language words and/or character sequences correspond to the set of features and are encoded or converted into corresponding feature vectors so that computers can process the corresponding extracted features. For example, a feature vector may represent a historical heat transfer fluid sample and its measured parameter values. “Feature space” or “vector space” as described herein refers to a set of all possible values (e.g., parameter values) for a chosen set of features (e.g., the six parameters described above) from the data.

In various embodiments, the machine learning model(s) 1105 learn, via training, parameters or weights so that similar features are closer (e.g., via Euclidian or Cosine distance) to each other in feature space. A “weight” in the context of machine learning represents the importance or significant of a feature or feature value for prediction. For example, each feature may be associated with a numerical or other real number where the higher the real number, the more significant the feature is for its prediction. In some embodiments, a weight in a neural network or other machine learning application can represent the strength of a connection between nodes or neurons from one layer (an input) to the next layer (an output). A weight of 0 may mean that the input will not change the output, whereas a weight higher than 0 changes the output. The higher the value of the input or the closer the value is to 1, the more the output will change or increase. Likewise, there can be negative weights. Negative weights proportionately reduce the value of the output. For instance, the more the value of the input increases, the more the value of the output decreases. Negative weights may contribute to negative scores. In an illustrative example of weighting in the context of neural networks, if most historical heat transfer fluid samples for a given label exhibited a range value X of parameter Y, the range value X may be associated with a neural node that is activated or strengthened.

In some embodiments, this training is done in supervised manner using a loss function (e.g. Triplet loss or GE2E loss) to determine how much a prediction deviates from the ground truth (e.g., labeled data sets). Loss functions learn to reduce the error in prediction. Training can occur on one or more of the data input(s) 1115, such as a labeled condition score heat transfer fluid sample, a labeled fluid life heat transfer fluid sample, and/or the labeled parameter analysis score (which represents the ground truth). In some embodiments, a “labeled condition score heat transfer fluid sample” represents one or more heat transfer fluid samples (e.g., within the data store 105) that have been labeled as “poor,” “fair”,” or “excellent,” which is reflective of whether or not the heat transfer fluid was in poor, fair, or excellent condition at the time of sampling. In some embodiments, a “labeled fluid life heat transfer fluid sample” represents one or more heat transfer fluid sample that have been labeled with different amounts of fluid life indicators (e.g., “less than two years remaining,” “between 2 to five years remaining,” and “five or more years remaining) at the time of sampling. In some embodiments, a “labeled parameter analysis score” represents one or more heat transfer fluid samples that have been labeled as “acceptable,” “marginal,” or “beyond limit”, as described herein with respect to the parameter analysis module 106 of FIG. 1. For each of these labels, various embodiments learn (e.g., via the historical heat transfer fluid sample analyzer module 101) weights associated with parameter values for each of these given labels. In some cases, multiple data points will be rationalized based on specific rules and therefore included or excluded for trending purposes

Some embodiments learn an embedding of feature vectors based on learning (e.g., deep learning) to detect similar features between training data input(s) 1115 in feature space using distance measures, such as cosine (or Euclidian) distance. For example, each labeled training data input 1115 is converted from string or other form into a feature vector where each value or set of values represents the individual parameter values. In some embodiments, feature space (or vector space) is a collection of feature vectors that are each oriented or embedded in space based on an aggregate similarity of features of the feature vector. Over various training stages or epochs, certain feature characteristics for each training input(s) 1115 can be learned or weighted. For example, for each of a plurality of labeled condition score heat transfer fluid samples labeled as “poor,” embodiments can learn that for heat transfer fluid Y (e.g., THERMINOL® 66), the most common factor was a parameter 1 value between X and Y, and a parameter 2 value of P, as represented in the output feature vector.

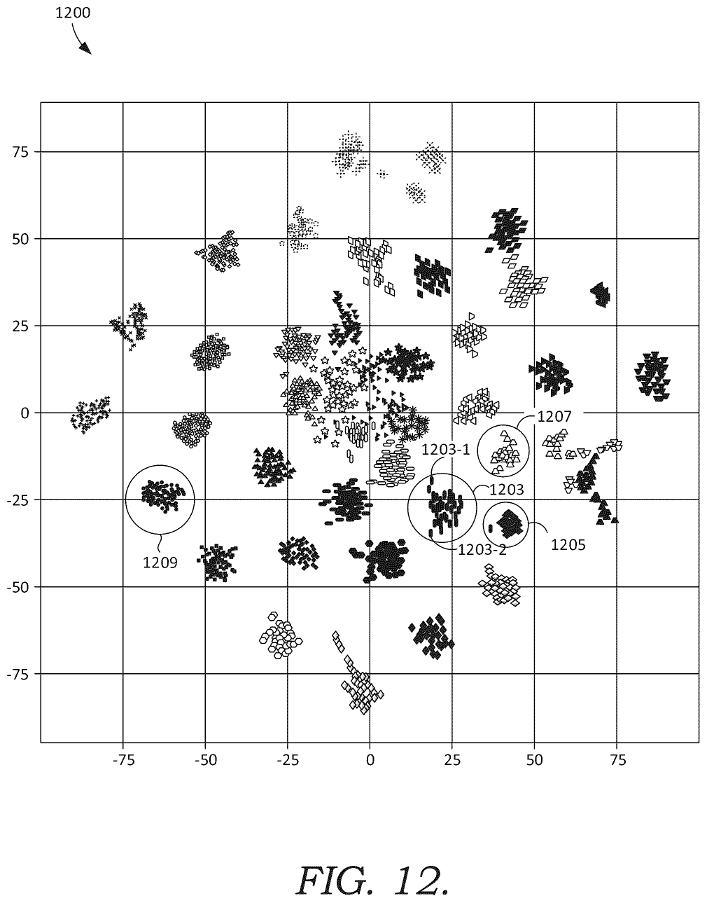

In some embodiments, learning or weighting includes changing an embedding in feature space of a feature vector representing historical heat transfer fluids as more training epochs occur. For example, after a first round or epochs of training, it may be unknown which of the extracted parameter values are important for taking on a certain classification or prediction. Accordingly, each feature may take on equal weight (or close to equal weight within a threshold, such as a 2% changed weight) such that all of the input feature vectors are substantially close or within a distance threshold in feature space. However, after several rounds of training or any threshold quantity of training, these same feature vectors may adjust or change distances from each other based on the feature value similarity. The more features of two feature vectors that match or are within a threshold value, the closer the two feature vectors are to each other, whereas when features do not match or are not within a threshold value, the further away the two feature vectors are from each other. Accordingly, for example, a trained embedding may look similar to the feature space 1200 of FIG. 12.

It is understood that while FIG. 11 illustrates that the training data inputs 1115 represent labeled inputs, as can be done in supervised learning, the inputs 1115 can represent non-labeled data, such as non-supervised clustering machine learning model data where no label is given. For example, some or all of the input(s) 1115 may not have labels because it is unknown what previous condition (e.g., “poor” or “excellent”), fluid life estimations, and/or parameter analysis (e.g., “acceptable” and “marginal”) scores were. In these embodiments, for example, models can learn a feature vector embedding to learn parameter value similarities between heat transfer fluid samples (or corresponding feature vectors) and cluster various historical heat transfer fluid samples together without regard to the labels.

Various embodiments convert the training data input(s) 1115 into a feature vector and map it in vector space by aggregating (e.g. mean/median or dot product) the feature vector values to arrive at a particular point in feature space. In other words, an embedded feature vector in vector space represents a particular heat transfer fluid sample that is oriented in vector space based on a combination of all of its parameter values, where each parameter value represents a dimension (or axis).

In various embodiments, subsequent to the machine learning model(s) 1105 training on the training data input(s) 1115 (and/or testing), the machine learning model(s) 11105 (e.g., in a deployed state) receives the runtime input 1103. In various embodiments, In some embodiments, the input 11003 is automatically converted to one or more feature vectors and mapped in the same feature space as vector(s) representing the training data input(s) 1115. Responsively, some embodiments determine a distance (e.g., a Euclidian distance or cosine) between the one or more feature vectors and other vectors representing the training data input(s) 1115, which is used to generate the decision statistic(s) 1107. For example, a first feature vector representing a new heat transfer fluid can be determined to be closest in distance to a feature vector labeled as “fluid life has less than 1 year remaining” or “poor.” In another example, a first feature vector representing an individual low boilers parameter of X heat transfer fluid can be determined to be closest in distance to another low boilers parameter of X heat transfer fluid that has an “beyond limit” label.

Responsive to the distance determinations between the feature vector representing the input 1103 and one or more of the training data inputs 1115, embodiments generate the decision statistic 1107. For example, using the illustrations above, based on the first feature vector representing a new heat transfer fluid being closest in distance to a feature vector labeled as “fluid life has less than 1 year remaining” or “poor,” the heat transfer fluid life can be predicted to be “less than 1 year remaining” or of “poor” in quality or condition. In another example, based on a first feature vector representing an individual parameter of X heat transfer fluid being closest in distance to another parameter of X heat transfer fluid that has an “beyond limit” label, the parameter of X heat transfer fluid is estimated to be at an “beyond limit” level (e.g., meaning that some corrective action needs to be taken to get it back to acceptable levels).

FIG. 12 is a schematic diagram of an example visualization of vector space 1200 that illustrates various clusters or classes of feature vectors, according to some embodiments. In some embodiments, the feature space 1200 represents the functionality produced (or used) by the heat transfer fluid life estimation module 104, the parameter analysis module 106, and/or the fluid condition estimation module 107 of FIG. 1, and/or the one or more machine learning models 1105 of FIG. 11.

In some embodiments, the vector space 1200 includes clusters of data points (e.g., data point 1203-1 and data point 1203-2) representing individual feature vectors corresponding to specific heat transfer fluid samples and/or individual parameters. These data points are formed together to form a particular class (or cluster). For example, the data point 1203-1 and data point 1203-2 may have been classified as “high quality heat transfer fluid X) 1203 (indicative that the feature values of the data points 1203 are within a threshold distance to or similar to other trained data points or that they refer to the exact same heat transfer fluid X of high quality). There are other classes, such as class 1205 (e.g., “heat transfer fluid X of low quality”) and the class 507 (e.g., “heat transfer fluid Y of low quality”).

In an illustrative example of how the feature space 1200 is used, embodiments may receive a historical heat transfer fluid sample, where all the parameters have been sampled. Responsively, some embodiments run the historical heat transfer fluid sample through one or more machine learning models in order to weight features for the sample, after which a feature vector (e.g., representing the data point 1203-1) is embedded in the feature space 1200. The feature space 1200 in various embodiments represents a multidimensional coordinate system where each feature (e.g., parameter) is associated with one or more dimensions. For example, a first set of values in a vector may represent a high boilers value of a first heat transfer fluid sample, where a first axis represents the first set of values and a second axis represents a second set of values of the same vector represents a low boilers value. Each feature value within the feature vector may be summed or otherwise aggregated to arrive at a final coordinate point (e.g., the data point 1203-2) within the feature space 1200. Each of the data points within the class 1203, for example, are within a feature similarity threshold and so they are close to each other (e.g., based on Euclidian distance) in the feature space 1200. Responsive to the embedding of the feature vector in the feature space 1200, embodiments classify or cluster the vectors. For example, if a first vector represents data point 1203-1, then the classification that is nearest to the data point 1203-1 is the “acceptable level of high boiler for heat transfer fluid Y” Classification 1203 indicative of the data point 1203-1 having a high boiler value that is of acceptable levels for heat transfer fluid Y.

The machine learning model(s) are able to cluster samples of new unseen heat transfer fluids (e.g., any heat transfer fluid received after training). In some embodiments, heat transfer fluids are represented by the median of its samples' embeddings as shown below:

C=median {fembed (Sij): I=1, 2, . . . , n]

Where fembed is the output of the model, Sij is the ith sample of the jth class. The prediction for any test sample X is given by:

Pred(X)=arg(min)_{T}j∥Cj−fembed (X)∥.

However, it is understood that median is just one way to represent an embedding. Some embodiments alternatively use other statistics like mean, pth percentile, and the like.

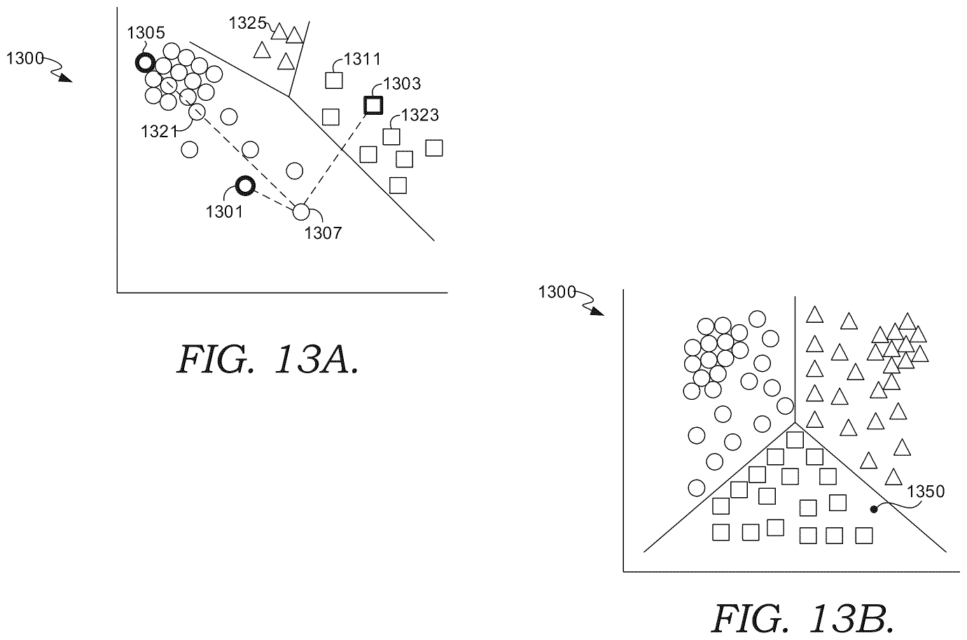

FIG. 13A is a schematic diagram of a cluster analysis visualization 1300 at a first time, according to particular embodiments. In some embodiments, FIGS. 13A and 13B represents the functionality performed by the heat transfer fluid life estimation module 104, the parameter analysis module 106, and/or the fluid condition estimation module 107 of FIG. 1. In some embodiments, FIG. 13A and FIG. 13B represents how each of the clusters with respect to FIG. 12 are generated.

The visualization 1300 of FIG. 13A includes a plurality of data points (e.g., data point 1307, data point 1321) that are clustered into three different groups represented by circles, squares, and triangles. The clusters and data points can represent any suitable value as described herein (e.g., parameter values (e.g., “acceptable,” “marginal,” or “beyond limit”) as illustrated in 517, 519, and 521 levels of FIG. 5A), quality (or condition) score estimates (e.g., 509 of FIG. 5A), or fluid life estimations (e.g., 505 of FIG. 5A)). For example, each data point can represent a particular parameter value of a same parameter type (e.g., a moisture content value) of a specific heat transfer fluid. In some embodiments, the visualization 1300 represents the clustering of data after only 1 or a few (e.g., 2-8) iterations. Prior to the first time, a user can select the quantity of clusters he or she wants to identify in the data. This is the “k,” value for example in k-means clustering. For example, the user can group data into a “acceptable” cluster, a “marginal” cluster, and an “beyond limit” cluster, where each cluster and data point is indicative of whether the corresponding parameter value is of a “acceptable” level, at a “marginal” level, or some immediate action needs to be performed to get back to acceptable levels (k=3) (e.g., as described above with respect to the parameter analysis module 106 of FIG. 1). In some embodiments, the k-value that is selected is determined by calculating the “elbow point” in a plot, which is a point at which variation between data points goes from a relatively large reduction in variation to minimal reduction in variation, as described herein.

Next, in various embodiments, the k value of distinct data points are randomly selected (e.g., by the one or more machine learning models 1105) as the initial clusters. For example, where k=3, the data points 1305, 1301, and 1303 can be selected as the initial clusters. Next, a distance can be measured (e.g., by the one or more machine learning models 1105) between a first point of a plurality of data points and each point of the initially selected clusters. For example, as shown in the visualization 1300, the data point 1307 is selected (e.g., as the “first point”), then the distance (e.g., Euclidian distance) between the data point 1307 and each of the initially selected clusters 1301, 1305, and 1303 is measured. Next, the first point is assigned (e.g., by the one or more machine learning models 1105) to the nearest of the initially selected clusters, such that two points are now within the same cluster. For example, the data point 1307 is assigned to the cluster or data point 1301, since the distance between the data point 1307 and 1301 is closer than the distance between data point 1307 and either of the two data points 1303 and 1305. Next, this process is repeated for each data point in the visualization 1300. For example, data point 1311 is selected. Then a distance is measured between data point 1311 and each of the three initially selected cluster data points 1305, 1301, and 1303. Because data point 1311 is closer, in distance, to the initial cluster data point 1303 than any of the other initially selected clusters 1301 and 1305, the data point 1311 is assigned to the cluster 1303 to belong to the same cluster or group.

In some embodiments, after each point of the plurality of points has been assigned to a cluster, the mean or center data point of each cluster is then calculated (e.g., by the one or more machine learning models 1105), which concludes a first round of clustering. Responsively, each center data point or mean is then used as initial data point clusters and the process described above is repeated for a second round of clustering. For example, the mean of a first cluster may be the data point 1321, the mean of a second cluster may be the data point 1325, and the mean of a third cluster may be data point 1323. Accordingly, a distance is measured between a first point (e.g., data point 1307) and each of the initially selected data point clusters 1321, 1325, and 1323. The first point is then assigned to the nearest of the three clusters. For example, data point 1307 can be assigned to the data point 1323 since it is the closest. This process is repeated for each of the data points in the visualization 1300 of FIG. 13A. In some embodiments, after this second round (or other quantity) of assigning data points to clusters and determining another mean value, it is determined (e.g., by the machine learning model(s) 1105) whether there have been clustering changes above a threshold. For example, it is determined whether the change in clusters between the first round and second round have changed outside of some threshold (e.g., the mean is plus or minus a particular value in difference between the first round and the second round). If there has been no change outside of a threshold, then the clustering process can conclude. However, if there is a change outside of the threshold, then particular rounds can be added until the clusters do not change outside of the threshold.

FIG. 13B is a schematic diagram of the cluster analysis visualization 1300 at a second time subsequent to the first time illustrated in FIG. 13A. FIG. 13B represents the changing of the clusters after one or more rounds or iterations according to the functionality described above with respect to FIG. 13A. As illustrated if FIG. 13B, the triangle cluster includes more data points or is larger compared to its cluster as represented in FIG. 13A. As also illustrated in FIG. 13B, each of the clusters are also in different orientations and cover different distances compared to their corresponding cluster in FIG. 13A. In this way, the clusters in FIG. 13B represent a more accurate depiction of clusters and therefore a likelihood that an incoming data point will be assigned to the correct cluster.

In some embodiments, the visualization 1300 (or any learning model described herein) of FIG. 13B represents 3 plotted clusters that categorize particular parameter values as “acceptable,” “marginal” or “beyond limit.” In this way, it can be predicted what cluster a parameter value belongs to for future analysis. For example, data point 1307 may represent a moisture content value Y for heat transfer fluid X. The data point 1301 may represent a second moisture content value P for another heat transfer fluid X. Continuing with this example, the first triangle-shaped clusters represent “acceptable” values, the second box-shaped clusters represent “marginal” values, and the third circle-shaped clusters represent “beyond limit” values. Accordingly, the distance between each data point may dependent on how similar each parameter value is. For example, moisture content values 31.40 and 31.41 may be grouped together under a “acceptable” cluster for heat transfer fluid Y. Continuing with the example above, the data point 1350 may represent a presently received parameter value. In this way, the parameter value can be placed in the corresponding cluster.

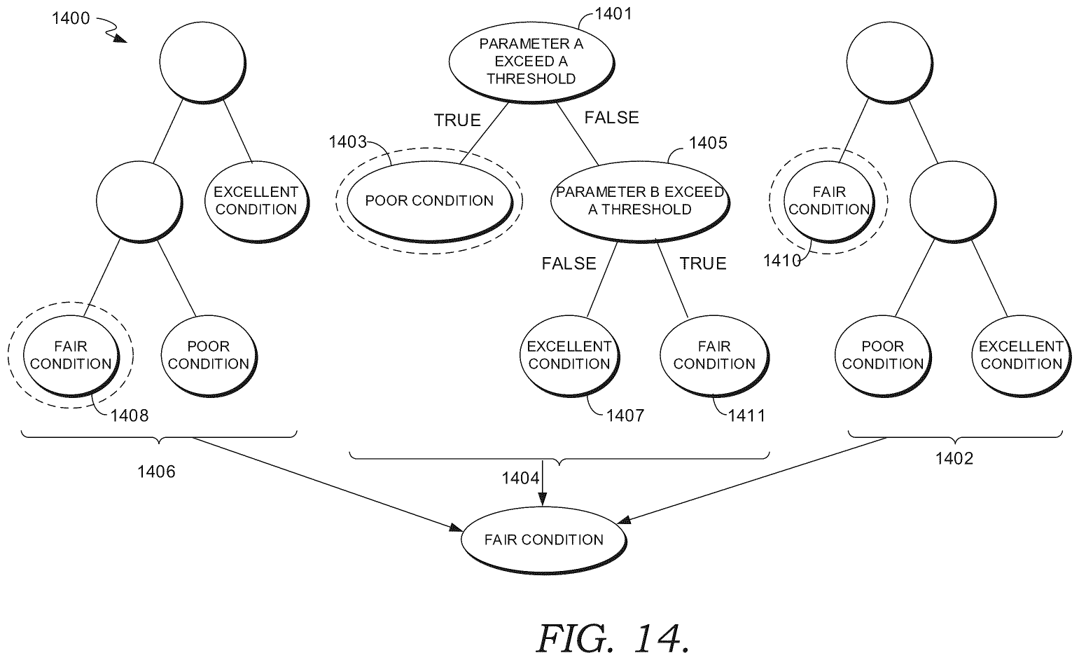

FIG. 14 illustrates an example random forest learning model 1400, according to particular embodiments. In some embodiments, FIG. 14 illustrates the model used by the fluid condition estimation module 107 of FIG. 1 and/or how the fluid condition score 509 of FIG. 5A is generated. Alternatively or additionally, some embodiments use identical or similar functionality for the heat transfer fluid life estimation module 104 and/or the parameter analysis module 106 of FIG. 1. Although FIG. 14 illustrates a specific random forest learning model, values with specific decision tree pathways, parameters, and tests, it is understood that any suitable value, node, test, and/or decision pathway may exist. It is also understood that although there is represented a specific quantity of decision trees with a particular quantity of nodes, there may be any suitable quantity of decision trees and corresponding nodes in the learning model. In various embodiments, the one or more machine learning models 1105 represent the random forest learning model 1400.

A random forest learning model includes various decision trees that each present random and unique decision pathway tests to arrive at the same set of results. More particularly, each decision tree within a random forest has at least one different root or branch nodes and tests but the same leaf node answers. Each decision tree is analyzed to determine which leaf node was traversed, as only one leaf node is traversed in particular embodiments. The leaf node with the highest quantity of traversals within the forest determines the output prediction (i.e., majority vote wins). Each root node or branch node includes a “test” corresponding to a question that determines whether a TRUE or FALSE pathway is traversed. For example, referring to the root node 1401, the test or question is whether the parameter A value of a particular heat transfer fluid exceeds a threshold. If yes or TRUE, then there is traversal to the node 1403, if no or FALSE, there is a traversal to node 1405 for further processing. Accordingly, the traversal of each decision tree starts at the root node, down through the branch nodes, until one of the leaf nodes are reached. The specific leaf nodes that are reached depends on answers to the given tests within the root and branch nodes. In various embodiments, each of these tests represent “rules” as described above that improve existing technologies in order to automatically predict or generate an estimate the quality (or fluid life) of a heat transfer fluid.

The learning model 1400 includes decision tees 1406, 1404, and 1402. Each decision tree has the same leaf node answers or values of “fair condition,” “poor condition,” and “excellent condition.” For example, the decision tree 1404 includes the leaf nodes 1403, 1407, and 1411, which represent “poor condition,” “excellent condition,” and “fair condition.” Identical leaf nodes are also indicated in the other decision tees 1406 and 1402. The learning model 1400 is used to generate an estimate of a quality score for a particular heat transfer fluid. Accordingly, the classifications of “excellent condition” “fair condition,” and “poor condition” are generated to reflect this prediction.

An example illustration of how each decision tree works is indicated by decision tree 1404. The historical heat transfer fluid sample data store 105 may include various sets of information that indicate when heat transfer fluids are in “excellent” “fair” or “poor” condition based on its parameter values and other rules that drive these determinations. Node 1401 is used for deciding whether a parameter A value exceeds a threshold (e.g., a predetermined action limit). If the parameter A value does exceed the threshold, then the “TRUE” pathway is traversed (e.g., a Boolean value is set to TRUE) and the system automatically classifies (e.g., indicates a high probability that) that the corresponding heat transfer fluid is of “poor condition”, meaning that the heat transfer fluid may need to be treated, replaced, or some other corrective action should be taken. However, if the parameter A value does not exceed the threshold, then the FALSE pathway is traversed, whereby another decision at node 1405 is made to determine if a parameter B value of the same heat transfer fluid exceeds some other threshold. If the parameter B value does not exceed the threshold, then the FALSE pathway is traversed and the heat transfer fluid is classified as being in “excellent condition” according to leaf node 1407. Alternatively, if the parameter B value does exceed the threshold, then the TRUE pathway is traversed and the heat transfer fluid is classified as being in “fair condition.” The decision tree 1404 illustrates that the “winning” leaf node is node 1403, indicating that the heat transfer fluid is in “poor condition” based on the specific key parameter values of a particular heat transfer fluid.

In various embodiments, the decision trees 1406 and/or 1402 include different branch and/or root nodes and tests compared to the decision tree 1404, but have the same leaf nodes. Accordingly, for example, decision tree 1406 can additionally or alternatively include a branch or root node that has a test labeled, “parameter C exceed a threshold.” If the parameter C value exceeds the threshold, then a TRUE pathway can be traversed in order to classify that particular heat transfer fluid is in a “poor” or “fair” condition. In another example, the decision tree 1402 may additionally or alternatively include a root and/or branch node test that is labeled “parameter D exceeds a threshold.” Accordingly, if the parameter D value exceeds the threshold, the heat transfer fluid may be classified as being in “poor” or “fair” condition, for example.

FIG. 14 also illustrates that the majority vote winner is the “fair condition” classification. Decision tree 1406 indicates that the heat transfer fluid has been classified as being in “fair condition” as indicated by the dotted lines around the leaf node 1408. However, the decision tree 1404 indicates that the same heat transfer fluid has been classified as being in “poor condition,” as indicated via the dotted lines around the leaf node 1403. The decision tree 1402 indicates that the same heat transfer fluid has been classified as “fair condition,” as indicated by the dotted lines around the leaf node 1410. Accordingly, the system tallies up the scores—there are 2 “fair condition” classifications compared to only 1 “poor condition” classification. Because the majority of decision trees indicate that the heat transfer fluid is in “fair condition” (2 compared to 1), the system predicts or estimates that the particular heat transfer fluid is of fair condition.

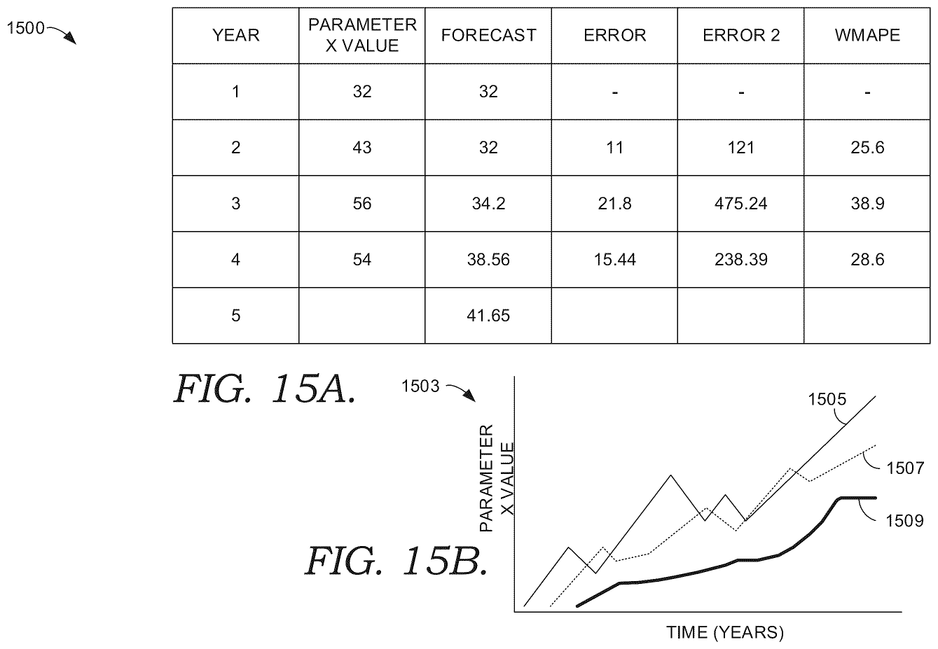

FIG. 15A is a schematic diagram of an example exponential smoothing forecast model table 1500, according to some embodiments. In some embodiments, the historical trend/forecast module 112 uses exponential smoothing as described herein to forecast particular parameter values (e.g., point 703-3 of FIG. 7). In some embodiments, such forecasts are additionally used to compute a condition score (e.g., as computed by the fluid condition estimation module), a heat transfer fluid life estimation (e.g., as computed by the heat transfer fluid life estimation module 104), and/or a parameter score (e.g., as computed by the parameter analysis module 106). Although the table 1500 includes specific values, calculations (e.g., WMAP), and time sequences (year 1-5), it is understood that this is representatively only and that any set of values, calculations, and/or time sequences can exist. For example, instead of or in addition to making volume forecasts for a particular set of “years,” there may be forecasts for a particular sequence of months, days, weeks, and/or any other time period sequence.

In another example, instead of or in addition to calculating WMAPE (weighted mean absolute percent error), other model accuracy validation methods can be used, such as root mean square error (RMSE), mean absolute percent error (MAPE), mean square error (MSE), and/or any other suitable error calculation mechanism. In various embodiments, the table 1500 (or similar table with the same calculations) is included in or used with one or more learning models, as described above with respect to the machine learning model(s) 1105. In some embodiments, the table 1500 represents a data structure stored in memory, such as a hash table. In some embodiments, the table 1500 is configured to be stored in memory and be displayed in response to or while generating output of a volume forecast.

The table 1500 illustrates what the parameter value forecast or prediction will be for years 1 through 5 for a particular parameter (e.g., viscosity). The particular values are populated within the table 1500 based on exponential smoothing forecast algorithms. In various embodiments, generating a forecast or prediction for a particular year is generated through the following expression: Ft+1=αAt (1−α)Ft, where Ft+1 is a particular forecast/prediction of a parameter value for a particular time period (year) or forecast/prediction for the current time period, where α (alpha) is a value between 0 and 1 (i.e., the smoothing constant), where Aₜ is the last actual parameter value (e.g., actual sampled parameter value) of the immediately preceding time period, and where Fₜ is the last forecast value (e.g., predicted parameter value at some future time) of the immediately preceding time period. For purposes of the specific values within the table 1500, alpha α is assumed to be 0.2.

In an example illustration, at year three it may be currently unknown what the parameter value will be for parameter X. However, the learning model may project that the parameter value will be 34.2 on year 3, as illustrated in the table 1500. Accordingly, using the expression above, the new forecast or forecast at year three (Ft+1)=(0.2)(43)+(0.8)(32), which equals 34.2. That is, alpha 0.2 is multiplied by the last actual value Aₜ of year 2, which is 43. The result is a value of 8.6. Then 0.8 (the value of 1−α) is multiplied by 32, which is the last forecasted value Ft of year 2 to arrive at a value of 25.6. Then 8.6 is added to 25.6 to arrive at the final result of 34.2. Accordingly, even though the current actual parameter value Aₜ of 56 may not be known at the time, it can be projected that it will be 34.2 at year 3. Then at a later time, the actual value for year 3 may be received, which is 56, which may be used to make future forecasts (year 4 and year 5). Year 5 illustrates a time period where the actual parameter value Aₜ is currently unknown, but the forecasted value Fₜ is still projected to be 41.65 based on using the expression above.

The “Error,” “Error2,” and “WMAP” columns of the table 1500 are utilized to validate accuracy of the exponential smoothing forecast model. The values of the “Error” column are calculated by subtracting the forecasted values from the actual values for each time period (At−Ft). For example, for year 2, Aₜ value of 43 is used to subtract the Fₜ year 2 value of 32 to arrive at an “Error” value of 11. The “Error2” values are calculated by squaring each of the corresponding Error values for the same time period. For example, for year 2, the error value of 11 is squared to arrive at a value of 121. The “Error2” column can be used to generate other analyses, such as MSE, which is calculated by adding up each squared error of the table 1500 and dividing this value by the total number of time periods (5 days). The “WMAPE” (weighted mean absolute percent error) is calculated via the following expression:

$$\frac{\sum \frac{\vert A - F \vert}{A} \times 100 \times A}{\sum A}$$

where A represents Aₜ or the current parameter value for a particular year and F represents Fₜ or the currently forecasted parameter value for the same particular year. For example, for year 2, the absolute value of 43 (the actual volume value)−32 (the forecasted parameter value) is divided by 43 to arrive at 0.256. This value is then multiplied by 100 and 43 to arrive at the value of 1, 100.0, which is then divided by 43 to arrive at the WMAPE value of 25.6 for year 2. WMAPE is utilized to focus on or weight errors that have a relatively larger impact or little to no impact at all. Standard MAPE calculations treat all errors equally, while WMAPE calculations place greater significance on errors associated with larger items by weighting these errors more.

FIG. 15B is a schematic diagram of an example time series graph 1503 associated with the table 1500 of FIG. 15A, according to some embodiments. The graph 1503 represents actual and forecasted parameter value predictions for different alpha values and actual values. In some embodiments, the “time” axis (X-axis) is or includes years 1-5 as indicated in the table 1500. For example, the “time” axis in the graph 1503 can represent a larger time sequence, such as years 1-20, where years 1-5 (as indicated in FIG. 15A) is only a portion of the overall time sequence. The “parameter X value” axis (Y-axis) represents the raw parameter values for parameter X. The time series instance 1505 represents the actual parameter value received over a first time at a particular trend or slope. The time series instance 1507 represents the projected parameter value that will be sampled over the same first time at a first alpha level (e.g., 0.7) at a particular trend. The time series instance 1509 represents the projected parameter value that will be sampled over the same first time at a second alpha level (e.g., 0.2) at a particular trend. As illustrated in the graph 1503, both the actual parameter values and the projections become considerably larger as the time progresses. In some embodiments, the graph 1503 is configured to be stored in memory and be displayed in response to generating output of a volume forecast (e.g., as indicated in the screenshot 700 of FIG. 7).

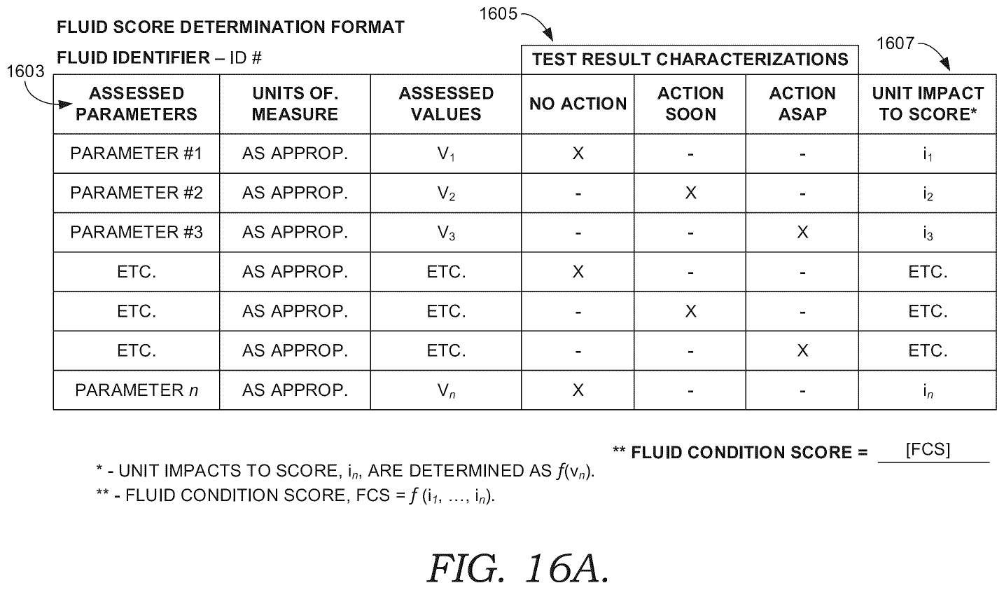

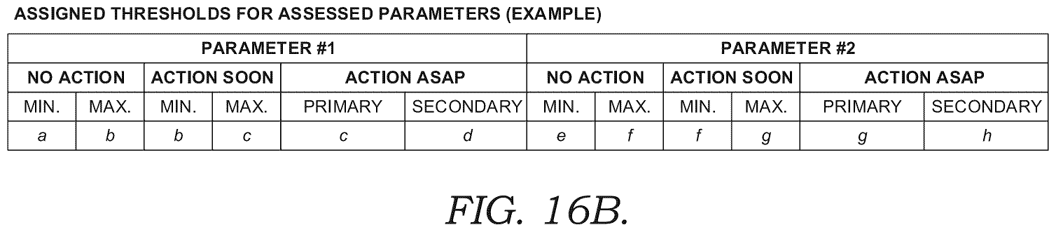

FIG. 16A is a schematic diagram illustrating how a heat transfer fluid quality score is generated, according to some embodiments. In some embodiments, FIG. 16A illustrates how the fluid condition estimation module 107 performs its functionality. In some embodiments, FIG. 16A represents how the fluid condition score 509 of FIG. 5A is generated. In some embodiments, FIG. 16A represents a screenshot rendered by the presentation component 114 of FIG. 1. In some embodiments, the objects, such as 1603, 1605, and 1607 represent data structures (e.g., hash maps) or other data objects used to store data.

The table attribute 1603 indicates each parameter used to generate a fluid quality estimation score. Each key parameter assessed of a fluid sample has a determined value and units of measure, which are used in determining the impact on fluid condition.

The table attribute 1605 indicates that the “assessed values” of table attribute 1607 are categorized as no action needed, action is needed soon, or action is needed as soon as possible (also referred to herein as “limits” or “thresholds”), as described herein with respect to the parameter analysis module 106. FIG. 16B illustrates how the different level thresholds can be assessed for the parameters, of FIG. 16A, for example. Accordingly, when a particular parameter value is at or falls within the particular category, a corresponding judgment is rendered on the parameter's condition for the fluid examined. For example, if a parameter value reads at 90 units and is within the “Action Soon” limit range of b to c, where b<90 units and c>90 units, then a the fluid is characterized as being in the “Action Soon” range. Similar thresholds can be determined for other parameters assessed. In some embodiments, these thresholds or levels are determined by the historical heat transfer fluid sample analyzer module 101, as described with respect to FIG. 1.

Returning back to FIG. 16A, the table 1607 indicates the particular heat transfer fluid sampled on all of the assessed parameter values for each parameter, example characterization of the assessed results and ultimately the unique impact the individual parameter has on the determined fluid condition score. Depending upon the characterized assessed values relative to the prescribed ranges defined in FIG.16B, the unique parameter impacts to fluid score are leveraged with multipliers of increasing value depending upon the degree of deviation of assessed value from “No Action” status. In some embodiments, the overall fluid score is determined as a compilation of the combined impacts of individual parameter impacts to a perfect fluid condition score of “Y”.

The object 1611 represents a heat map that visually indicates the fluid score. For example, the closer the value is to 0, the more red the color is indicating “Action Soon” or “action ASAP” values and the closer the value is to Y, the more green the color is indicating “No Action” values.

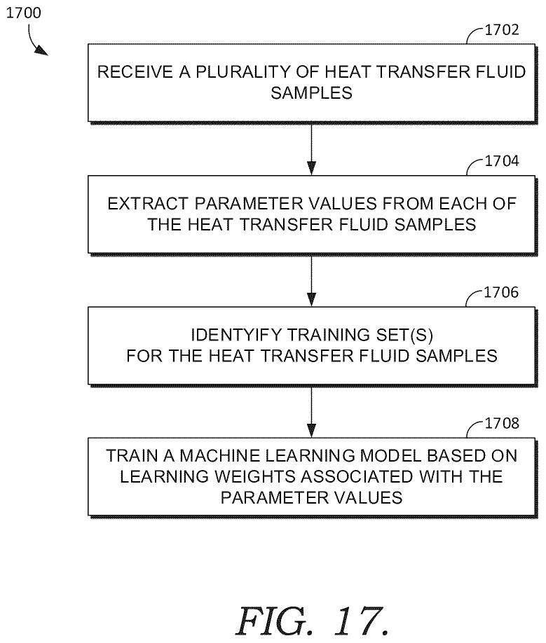

FIG. 17 is a flow diagram of an example process 1700 for training a machine learning model using heat transfer fluid samples, according to some embodiments. In some embodiments, the process 1700 is performed to train the machine learning model(s) 1105 of FIG. 11, the random forest model 1400 of FIG. 14, the clustering model of FIGS. 13A and 13B, or any machine learning model described herein. Alternatively or additionally, in some embodiments, the process 1700 includes or represents the functionality for training as described with respect to the training data inputs 1115 of FIG. 11. In some embodiments, the process 1700 is used or performed by the historical heat transfer fluid sample analyzer module 101 to determine patterns or relationships among heat transfer fluid samples.

The process 1700 (and/or any of the functionality described herein) may be performed by processing logic that comprises hardware (e.g., circuitry, dedicated logic, programmable logic, microcode, etc.), software (e.g., instructions run on a processor to perform hardware simulation), firmware, or a combination thereof. Although particular blocks described in this disclosure are referenced in a particular order at a particular quantity, it is understood that any block may occur substantially parallel with or before or after any other block. Further, more (or fewer) blocks may exist than illustrated. Added blocks may include blocks that embody any functionality described herein (e.g., as described with respect to FIG. 1 through FIG. 16B). The computer-implemented method, the system (that includes at least one computing device having at least one processor and at least one computer readable storage medium), and/or the computer readable medium as described herein may perform or be caused to perform the process 1700 or any other functionality described herein.

Per block 1702, a plurality of heat transfer fluid samples are received. In some embodiments, these heat transfer fluid samples represent the heat transfer fluid samples as stored to the historical heat transfer fluid sample data store 105 of FIG. 1 In some embodiments, such in a supervised machine learning context, each heat transfer fluid sample has been labelled at the dataset generation stage to indicate the ground truth. In some embodiments, each heat transfer fluid sample is identified by customers or other users (e.g., people who do not build machine learning models) prior to training. In this way, building a model does not require extensive labelling by model implementers. For example, some embodiments are naturally supervised and may receive user-defined heat transfer fluid samples with indications of whether each of the parameters fell within “acceptable,” “marginal,” or “beyond limit” ranges, whether the heat transfer fluid quality score was “fair,” “poor,” or “excellent,” and/or the particular heat transfer fluid life estimation (which may also be the labels in a supervised context).

Per block 1704, particular embodiments extract parameter value feature(s) from each transfer fluid sample, of the plurality of heat transfer fluid samples. For example, various embodiments extract any one of the parameters and measured values as indicated in FIG. 16A, such as “moisture content, 100,” “acid number, 0.1,” “viscosity, 20.3” and the like.

Per block 1706, one or more training sets are identified for the heat transfer fluid samples. For example, in a supervised context where the same heat transfer fluid sample types (e.g., THERMINOL® 55) have the same labels (e.g., “excellent” fluid quality score), these can be paired for training. In another example, different labels (e.g., “excellent” and “poor”) of the same heat transfer fluid samples can also be used. In yet another example, different heat transfer fluid types (e.g., THERMINOL® 55 and water) can also be paired for training.

Per block 1708, a machine learning model (e.g., a deep learning model) is trained based at least in part on learning weights associated with the parameter values. For example, it can be learned that the most significant factors for determining whether a particular heat transfer fluid is of “poor” quality is a particular range of high boilers value, a particular range of moisture content value, and a particular range of acid number values, whereas all of the other parameter values or highly variable and there is no significant pattern detected. In some embodiments, pairs of heat transfer fluid samples are processed or run through a deep learning model by mapping them in feature space based on their parameter values. And based at least in part on the processing, weights associated with the deep learning model can be adjusted to indicate the importance of the extracted parameter values for prediction or classification.

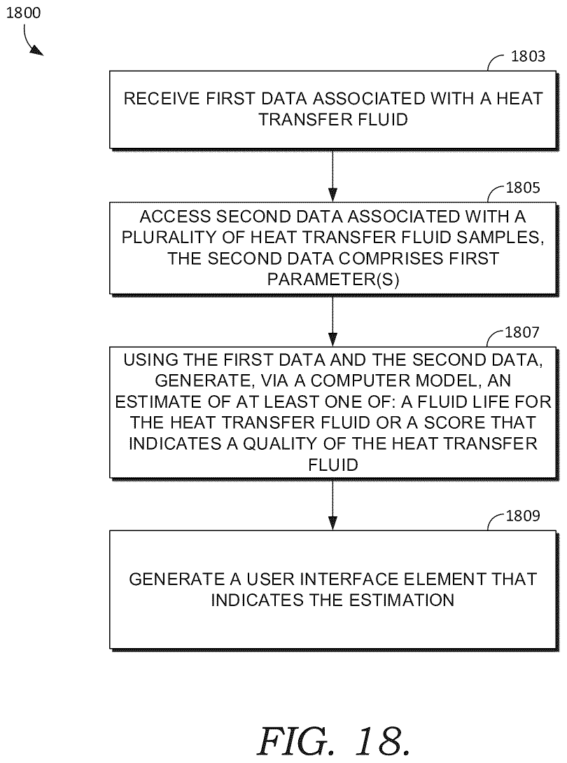

FIG. 18 is a flow diagram of an example process 1800 for generating one or more estimates associated with a heat transfer fluid, according to some embodiments. Per block 1803, first data (or second data) associated with a heat transfer fluid is received. Examples of block 1803 are described with respect to the “new” heat transfer fluid received by the heat transfer fluid registration module 102 of FIG. 2. In some embodiments, the first data represents the runtime input 1103 of FIG. 11.

In some embodiments, block 1803 is preceded by prompting a user to obtain a sample kit, receiving a sample of the heat transfer fluid, and subsequently extracting one or more parameter values associated with the first data. Examples of this are described with respect to FIG. 2, FIG. 9A, FIG. 9B, and the heat transfer fluid registration module 102 of FIG. 1. In other words, the received first data may correspond to a heat transfer fluid whose parameter values have already been (or will be) extracted or measured (e.g., based on a user sending in a sample via a sample kit). In some embodiments, such “one or more parameters” (e.g., a second plurality of parameters) include any parameters and parameter values as described herein, such as those parameters indicated in in FIG. 16A, FIG. 5A, and the like (e.g., high boiler values, viscosity values, moisture content values, etc.).

In addition to block 1803, some embodiments additionally convert the first data into a feature vector and map the feature in vector space. Examples of this are described with respect to FIG. 11 and the visualization 1200 of FIG. 24, where one or more machine learning models can embed feature vectors representing heat transfer fluid samples into vector space.

Per block 1805, various embodiments access second data associated with a plurality of heat transfer fluid samples, where the second data comprises one or more first parameters. Examples of block 1805 are described with respect to FIG. 1 where the historical heat transfer fluid samples are accessed in the historical heat transfer fluid sample data store 105 (e.g., stored in computer memory) by the heat transfer fluid life estimation module 104, the parameter analysis module 106, and/or the fluid condition estimation module 107. In some embodiments, the second data excludes (or does not include) the first data. For example, the historical heat transfer fluid sample data store 105 may exclude any new heat transfer fluids registered by the heat transfer fluid registration module 102 or within the registration data store 103. In some embodiments, each heat transfer fluid sample, of a plurality of heat transfer fluid samples, is associated with a plurality of parameters (e.g., heat transfer fluid having measured vales as indicated in the tables 1603 and 1607 of FIG. 16A).

In some embodiments, the one or more first parameters (or the plurality of parameters) described herein refers to one or more parameter values of a group of parameter values consisting of: a high boilers value, a low boilers value, a moisture content value, an acid number value, an insoluble solids value, a viscosity value, a carbon residue value, a flash point value, a non-evaporable content value, a contamination value (e.g., contamination present, yes or no), and a mixture value (heat transfer fluid sample is a mixture or not a mixture). Each of these parameters are described with respect to the table 1603 and 1607 of FIG. 16A, and with respect to a description of parameters associated with FIG. 5A.

Per block 1807, using the first data and the second data, and/or 3^{rd} and/or 4^{th} etc., various embodiments generate, via an empirical and non-empirical computer model, at least one of: an estimate of a fluid life for the heat transfer fluid or a score that indicates a quality of the heat transfer fluid. Examples of block 1807 are described with respect to the “fluid life remaining” score 505 and the “fluid condition score” 509 of FIG. 5A. Other examples include the functionality described with respect to the heat transfer fluid life estimation module 104 and the fluid condition estimation module 107. In some embodiments, the “computer model” described herein includes at least one of a statistical model or a machine learning model or a combination of the two, such as a hybrid model that combines time series regression (TSR) statistical methods with machine learning models, such as Feedforward Neural Networks (FNN). A machine learning model includes an algorithm that learns from data without relying on rules-based programming. Statistical models formalize relationships between variables via mathematical equations. Typically statistical models do not learn or train, but in some instances machine learning functionality includes statistical model functionality (e.g., random forest regression, as illustrated in FIG. 14). Examples of machine learning models include the machine learning model(s) 1105 of FIG. 11 (e.g., neural networks), and the process 1700 of FIG. 17. Examples of statistical models include linear regression, logistics regression, clustering, classifying, Bayesian statistics (e.g., FIG. 13A, FIG. 13B, FIG. 15A, and FIG. 15B).

In some embodiments, block 1807 is preceded by converting the accessed data into a set of feature vectors and mapping those feature vectors in vector space, where each feature vector of the set of feature vectors represent a corresponding heat transfer fluid sample, and the sample's parameter(s). This is described with respect to the FIG. 11, and the visualization 1200 of FIG. 12. Accordingly, in some embodiments, block 1807 represents or includes computing, via one or more models, a distance, in the vector space between at least one of a first set of feature vectors (e.g., representing historical heat transfer fluid samples) and a second feature vector (e.g., representing a new heat transfer fluid). And based at least in part on the computing, performing the estimating of the fluid life and/or quality score for the heat transfer fluid. Examples of this are described with respect to FIG. 11, and the visualization 1200 of FIG. 12. For example, referring back to FIG. 11, a distance (e.g., Euclidian or Cosine) between a feature vector representing the runtime input 1103 and one of the training data inputs 115 can be computed to arrive at the decision statistic 1107. Such decision statistic can then be mapped (e.g., via a data structure, such as a hash map, or classifier) to the particular fluid life estimation or fluid condition score.

In some embodiments, the “distance” is based at least in part on values of parameters indicated in a feature vector (e.g., representing a new heat transfer fluid) relative to other values of parameters of heat transfer fluid samples. As described herein, for example, each value indicated in a feature vector representing parameter values can be aggregated or concatenated (e.g., via a mean or dot product) to arrive at a final embedding in feature space.

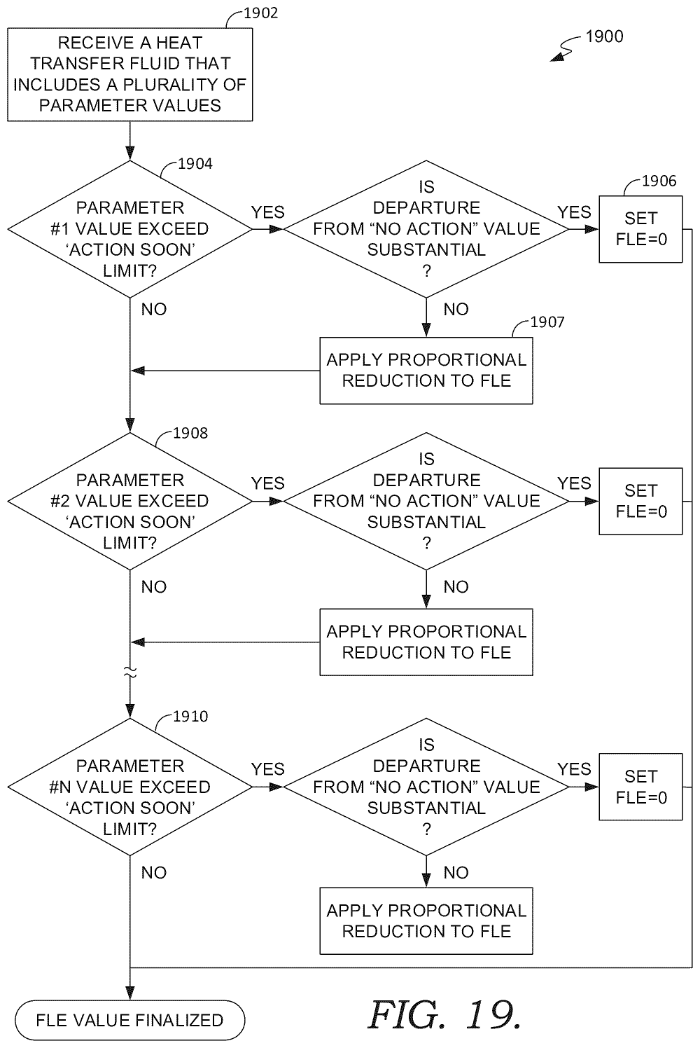

Alternatively or additionally, in some embodiments, where the first data includes one or more second parameters (e.g., that share at least one parameter attribute (e.g., high boiler) with the one or more first parameters), the estimate is based at least in part on determining whether a value of the one or more second parameters exceeds one or more predetermined thresholds associated with the plurality of heat transfer fluid samples. Examples of this are described with respect to FIG. 14, for example, where the 126 value of the insoluble solids parameters exceeds a predetermined threshold (e.g., 125), such that the “marginal” score is given, as indicated in the table 1605. This is used to compute the overall fluid score 1609. Other examples of this are described with the conditional rules, logic, or tests (e.g., FIG. 14), where one or more threshold values are compared against parameter values to determine a score (e.g., “fair” condition). FIG. 19 also illustrates this, as described in more detail below.

In some embodiments, the estimate (e.g., fluid life or quality score) of block 1807 is further based on training one or more machine learning models using the plurality of heat transfer fluid samples. Examples of this are described with respect to FIG. 11 and the process 1700 of FIG. 17. For example, referring back to FIG. 11, the decisions statistic 1107 is generated based at least in part on the training data inputs 1115 of FIG. 11. In another example, using the illustration above for FIG. 17, after training on the labels (e.g., the ground truth) for “acceptable,” “marginal,” or “beyond limit” ranges, heat transfer fluid quality score of “fair,” “poor,” or “excellent,” and/or the particular heat transfer fluid life estimation, a pattern can be detected where the moisture value was between A and B for heat transfer fluid C for “acceptable” levels of moisture values, “excellent” fluid quality scores, and/or fluid life estimations of “5 years remaining.” Accordingly, for example, if a new heat transfer fluid C had a moisture value between A and B, then it can predicted that it has “acceptable” moisture values, “excellent” fluid quality, and/or that there is 5 years remaining for heat transfer fluid C.

In some embodiments, the estimate of the fluid life or quality of the heat transfer fluid is further based on weighting the one or more second parameters based on information derived from the plurality of heat transfer fluid samples. “Weighting” is described with respect to the “weight factor” of FIG. 14, a “weight” with respect to FIG. 11 or neural networks, and/or the fluid condition estimation module 107 of FIG. 1. For example, referring back to FIG. 14, the weighting factor for parameter 1 content can be used to estimate the fluid score 1609 and the Y value can be derived from patterns identified (e.g., by the historical heat transfer fluid sample analyzer module 101) within historical heat transfer fluid samples. For example, the Y weight factor can be indicative of a high or heavy weight based on moisture content being an important factor for determining the quality and/or fluid life of any heat transfer fluid (as opposed to a lower weigh factor X).

In some embodiments, block 1807 is based on or is succeeded by the following functionality. Some embodiments determine whether a value of the one or more second parameters falls within an acceptable range, and wherein the estimating is based at least in part on this determination. And based on this determining, some embodiments generate a second user interface element that indicates whether the value of the one or more second parameters falls within the acceptable range. Examples of this are described herein, such as the UI elements 515 (e.g., and via the level indicators 517, 519, and 521). This functionality is also described with respect to the parameter analysis module 106 and fluid condition estimation module 107 of FIG. 1.

In some embodiments, the following functionality precedes or succeeds block 1807. For instance, some embodiments generate historical trend data of at least one parameter, of a second plurality of parameters associated with the heat transfer fluid. And in response to the generating of the historical trend data, particular embodiments caused display of the historical trend data and an identifier that indicates the at least one parameter. Examples of this are described with respect to the historical trend/forecast module 112 of FIG. 1, FIG. 7, FIG. 15A, and FIG. 15B.

Per block 1809, some embodiments generate a user interface element that indicates the estimation. Examples of this are described with respect to the presentation component 114 of FIG. 1, and the various screenshots of FIGS. 2 through 10. For example, embodiments can generate the user interface elements 503, 505, 507, and/or 509 of FIG. 5A, which indicate the fluid life (fluid life remaining) and fluid quality (fluid condition score) of a particular heat transfer fluid. In some embodiments, block 1809 is based at least in part on the computing of the distance described herein. For example, each distance (e.g., Euclidian or Cosine) can be mapped (e.g., via a hash map) to a corresponding fluid life or fluid quality estimation so that the estimation can be outputted to a user interface (alternative or in addition to the distance). In some embodiments, the generating of the user interface element (e.g., 505) occurs in response to the generation of the estimate. In other words, the estimation is first computed and then rendered for display in some embodiments.

In some embodiments, block 1809 is preceded or succeeded by generating a recommendation signal that indicates instructions for how to service the heat transfer fluid (or the heat transfer fluid system that contains the heat transfer fluid) based at least in part on the estimate exceeding a threshold. Alternatively or additionally, some embodiments generate a recommendation signal that recommends a change of the heat transfer fluid. Alternatively or additionally, some embodiments generate a recommendation signal of a corrective action to take (or cause a corrective action to occur). All of this functionality is described with respect to the maintenance recommendation module 108 of FIG. 1, FIG. 6, FIG. 8A, and FIG. 8B, as well as a description of the “corrective actions” that can be taken, as described with respect to the UI element 515 of FIG. 5A.

FIG. 19 is a flow diagram of an example process 1900 for estimating a life remaining of a heat transfer fluid, according to some embodiments. With respect to FIG. 19, such estimation is indicated as a “fluid life” (FL) or other examples can include fluid life remaining or fluid useful life score. In some embodiments, the process 1900 represents the functionality performed by the heat transfer fluid life estimation module 104 of FIG. 4. Likewise, in some embodiments, the process 1900 is performed to generate the “fluid life” score 505 of FIG. 5A. In some embodiments, FL is expressed in terms of remaining years and/or months of expected usable fluid life. Alternatively or additionally, FL can be expressed in terms of remaining weeks and/or days. In some embodiments, the FL score is calculated based upon the sample's existing elapsed life as measured by its degree of degradation, the measured thermal degradation rate of the fluid's specific chemistry as quantified by analysis per ASTM D-6743, and is dependent upon the extent of operation at the fluid's maximum operating temperature extreme, among other factors. Units of measure can be years/months remaining until or more key parameters reach prescribed “Action ASA” threshold(s). Criteria are also applied to assessed parameters to determine deteriorated life and/or quality in case adjustments are necessary. In this way, for example, an FL score can be determined based on established key parameters when properly assessed, as illustrated below with respect to FIG. 19. The process 1900 describes various parameter values of a heat transfer fluid being compared against various threshold values in order to determine a fluid life score.

Per block 1902, a heat transfer fluid that includes a plurality of parameter values is received. The plurality of key parameters can include value assessed typically from six to ten, but not limited to, measures of thermal degradation, viscosity, acid number, base number, pH, moisture, flash point, degree of contamination, solvent insoluble solids, bromine index value, autoignition temperature, and specific gravity.

In some embodiments, the heat transfer fluid is a new heat transfer fluid, as described with respect to the heat transfer fluid registration module 102 of FIG. 1. In each parameter assessed, industry accepted test methods can be preferred, such as ASTM-93 Standard Test Methods for Flash Point by Pensky-Martens Closed Cup Tester and ASTM D-664 Standard Test Method for Acid Number of Petroleum Products by Potentiometric Titration, as examples.

Upon receipt of the fluid sample assessed parameters, per block 1904, it is determined whether the Parameter #1 assessment value exceeds “Action Soon” limit (e.g., a predetermined threshold for the Parameter) (e.g., 28.0%). In some embodiments, this “Action Soon” maximum limit refers to the “action” score or levels described herein, such as with respect to the parameter analysis module 106 of FIG. 1 and/or FIG. 5A. For example, various parameters as indicated in the UI element 514 of FIG. 5A may have values that meet or exceed a “acceptable” level 521, a “marginal” level 519, or an “action” level 517. Such “action” level 517 may correspond to the “Action Soon” limit as described with respect to FIG. 19. As described herein, each parameter may be associated with their own action limit or threshold value (e.g., dependent on the pattern detected by the historical heat transfer fluid sample analyzer module 101).

Per block 1906, if the Parameter #1 assessed value exceeds the “Action Soon” maximum limit substantially (e.g., the value is 14.1%), the FL score is set to 0 and a corresponding notification is rendered. When an FL score is at or near 0, this means that there is no time or little time remaining in a fluid life or end life. That is, its life left is at 0 because there are no years, months, days, left. In other words, the heat transfer fluid is considered no longer suitable for use (e.g., has compromised ability to provide indirect process heat transfer compared to a new, unused same or similar heat transfer fluid). In some embodiments, in response to performing block 1906, a notification can be sent, which reads “please consult your area Fluid Specialist to discuss a replacement plan for your system fluid” (e.g., as generated by the maintenance recommendation module 108).

If the deviation of the assessed value is not substantial (e.g., the value is 8.1%), then, per block 1907, the FL is reduced an amount proportional to the degree of deviation from “No Action” range, according to a consistently applied and prescribed methodology based upon practical experience. This is known as the ‘Initial Score”. In some embodiments, a deviation is not considered substantial if the deviation is not greater than 1.1× to 4× the “Action Soon” maximum value for the specific fluid and Parameter evaluated.

Per block 1908, if the Parameter #1 value does not exceed the “Action Soon” limit (e.g., the Parameter's value is 1.6%), then the FL score is proportionally reduced to the remaining time period until the Parameter #1 value meets (or is projected to exceed (e.g., such as via the forecasting model 15A and 15B)) the “Action Soon” maximum limit. In some embodiments, this only occurs if all other heat transfer fluid life parameter values are in below their respective “Action Soon” maximum limits.

Block 1910 represents each successive Parameter and corresponding value that has been assessed. Per block 1910, it is determined whether the Parameter value has exceeded its respective “Action Soon” maximum limit. Similar to the evaluation in blocks 1904 and 1908, if the value has exceeded the maximum “Action Soon” maximum limit, the FL may be set equal to zero, or alternatively set to approximately zero. Logic is provided for FL values to be set to approximately zero in cases where the fluid may be temporarily unsuitable for continued use at its intended operating temperature, and when recommended treatment is expected to restore the fluid's suitability for use. An example of this instance is when the Parameter is moisture content, and the assessed value is excessively high, such as 10,000 ppm. At such a high moisture concentration, the fluid is completely unsuitable for use at operating temperatures above 120 C, for example, due to extreme pressure generated from vaporizing moisture, and from the accompanying pump cavitation induced from vaporizing water at the intake of the pump. In such cases, the FL is set to approximately zero and a notification is provided for the excess moisture to be vented from the system as a corrective action. Effectively performing the venting action would be expected to restore the fluid to a condition suitable for use as intended. Subsequent sample analysis would be expected to result in an assessed moisture content restored to within its “No Action” concentration ranges (e.g., from FIG. 16B), and the fluid life expectancy (FL) would be once again restored to a value (e.g., years/months) representative of the moisture content never having been excessive. As was the case in block 1907, if the deviation of the Parameter is not considered substantial, the reduction in FL is measured and proportional to the degree of deviation assessed from “No Action” range.

Continuing with the process 1900 provided in FIG. 19, once all of the key Parameters' assessed values have been considered and after the accumulated reductions of Fluid Life (FL) from each Parameter have been imposed, the final Fluid Life (FL) determination is completed and provided for use at output from module 104 (FIG. 1).

For any Parameters having assessed values exceeding their respective “Action Soon” maximum values, a notification for recommended corrective actions may be communicated. In some embodiments, by selecting UI element 511 (FIG. 5A) to view “Improve Fluid Life” opportunities, the Initial Fluid Life Expectancy (FLE) score will be displayed as the potential improvement possible by effectively performing the recommended corrective actions. In response to receiving an indication that filtering has been done, particular embodiments use the initial FLE score or “initial score”, as described herein, as the potential FL possible to achieve.

FIG. 20 is a flow diagram of an example process 2000 for modifying a fluid life expectancy and/or a fluid condition score based on a heat transfer fluid system being serviced, according to some embodiments.

Per block 2002, some embodiments determine that one or more parameter values of a heat transfer fluid do not meet (e.g., falls outside of, is below, exceeds, or does not exceed) one or more thresholds. For example, some embodiments can determine that a moisture content level exceeds a threshold. The one or more parameter values can be any parameter value described herein, such as a specific gravity value, a color value, a high boilers value, a base number value, value, an opacity value, a low boilers value, a pH value, a moisture content value, an absorbance value, an acid number value, a bromine index value, an insoluble solids value, a degradation value, a viscosity value, a carbon residue value, an autoignition temperature value, a flash point value, a non-evaporable content value, a conductivity value, a contamination value, an operating temperature value, an on-stream time value, and a mixture value.

Per block 2004, based at least in part on the determining at block 2002, particular embodiments generate a recommendation signal indicating instructions to service the heat transfer fluid (or system that contains the heat transfer fluid). For example, referring back to FIG. 5 and FIG. 6, the fluid life 505 (e.g., 1 year remaining) may be below a fluid life expectancy threshold (e.g., 2 years remaining) and/or the fluid quality 509 (e.g., “low” quality) may be below a fluid quality threshold (e.g., “medium” quality). Responsively, particular embodiments cause presentation of indicia 603 of FIG. 6 in order to recommend that maintenance action X be taken to service the heat transfer fluid.

Per block 2006, based at least in part on the generating of the recommendation signal at block 2004, particular embodiments modify a Fluid Life Expectancy (FLE) and/or Fluid Condition Score (FCS), where the modifying is indicative of what the FLE and/or FCS would be if the service to the heat transfer fluid was performed. In some embodiments, the “modifying” of the FLE and/or FCS includes or alternatively represents generating a separate FLE and/or FCS. For example, the FLE score 505 and/or the FCS 509 may still be present, as well as the FLE score 620 and the FCS 622 such that the scores 505 and 509 are not modified. Rather, separate scores 620 and 622 are generated as hypothetical scores had the user performed maintenance action X.

In some embodiments, the modifying (or otherwise generating) of the FLE and/or FCS scores at block 2006 occurs via the functionality as described with respect to the neural network 1105 of FIG. 11, the clustering of FIGS. 13A and 13B, the Random Forest models of FIG. 14, and/or the process 1700 for training a model. For example, in some embodiments, one or more machine learning models learns a mapping for specific maintenance actions taken and corresponding life expectancy scores or fluid condition scores. For example, training data may indicate that for a first set of heat transfer fluid samples of type A where maintenance action X was taken, the actual fluid life or condition was C (or within a range threshold of C). The training data may further indicate that for a second set of heat transfer fluid samples of type A, where maintenance action F was taken, the actual fluid life or condition was E (or within a range threshold of E). Accordingly, for a currently analyzed heat transfer fluid type A, if it is recommended that maintenance action X be taken, the predicted fluid life or condition may be C (instead of E) based on the historical heat transfer fluid samples' fluid life or condition readings for the same maintenance actions taken with respect to the same heat transfer fluid type.

Alternatively or additionally, one or more programmatic rules or policies may be implemented to map, via a data structure, the specific maintenance actions and life expectancy scores (or fluid condition scores). For example, a lookup table or hash map can be created where there the first column includes maintenance action values used as a key to look up corresponding life expectancy scores. For example, a first record can include a key that indicates maintenance action X, as well as a corresponding value Y, which indicates a specific life expectancy scores. A second record can include another key that indicates maintenance action Z, as well as corresponding value J, which indicates another life expectancy score.

FIG. 21 is a block diagram of a computing environment 2100 in which aspects of the present disclosure are employed in, according to certain embodiments. Although the environment 2100 illustrates specific components at a specific quantity, it is recognized that more or less components may be included in the computing environment 2100. For example, in some embodiments, there are multiple user devices 2102 and multiple servers 2104, such as nodes in a cloud or distributing computing environment. In some embodiments, some or each of the components of the system 100 of FIG. 1 are hosted in the one or more servers 2104. In some embodiments, the user device(s) 2102 and/or the server(s) 2104 may be embodied in any physical hardware, such as the computing device 2200 of FIG. 22.

The one or more user devices 2102 are communicatively coupled to the server(s) 2104 via the one or more networks 110. In practice, the connection may be any viable data transport network, such as, for example, a LAN or WAN. Network(s) 110 can be for example, a local area network (LAN), a wide area network (WAN) such as the Internet, or a combination of the two, and include wired, wireless, or fiber optic connections. In general, network(s) 110 can be any combination of connections and protocols that will support communications between the control server(s) 2104 and the user devices 2102.

In some embodiments, a user issues a query on the one or more user devices 2102, after which the user device(s) 2102 communicate, via the network(s) 110, to the one or more servers 2104 and the one or more servers 2104 executes the query (e.g., via one or more components of FIG. 1) and causes or provides for display information back to the user device(s) 2102. For example, the user may issue a query at the user device 2102 that is indicative of a request to provide a heat transfer fluid life or quality score. Responsively, the server(s) 2104 can perform functionality necessary to generate the scores.

The invention may be described in the general context of computer code or machine-useable instructions, including computer-executable instructions such as program modules, being executed by a computer (or one or more processors) or other machine, such as a personal data assistant or other handheld device. Generally, program modules including routines, programs, objects, components, data structures, etc., refer to code that perform particular tasks or implement particular abstract data types. The invention may be practiced in a variety of system configurations, including hand-held devices, consumer electronics, general-purpose computers, more specialty computing devices, etc. The invention may also be practiced in distributed computing environments where tasks are performed by remote-processing devices that are linked through a communications network.

With reference to FIG. 22, computing device 2200 includes bus 10 that directly or indirectly couples the following devices: memory 12, one or more processors 14, one or more presentation components 16, input/output (I/O) ports 18, input/output components 20, and illustrative power supply 22. Bus 10 represents what may be one or more busses (such as an address bus, data bus, or combination thereof). Although the various blocks of FIG. 22 are shown with lines for the sake of clarity, in reality, delineating various components is not so clear, and metaphorically, the lines would more accurately be grey and fuzzy. For example, one may consider a presentation component such as a display device to be an I/O component. Also, processors have memory. The inventors recognize that such is the nature of the art, and reiterate that this diagram is merely illustrative of an exemplary computing device that can be used in connection with one or more embodiments of the present invention. Distinction is not made between such categories as “workstation,” “server,” “laptop,” “hand-held device,” etc., as all are contemplated within the scope of FIG. 22 and reference to “computing device.”

In some embodiments, the computing device 2200 represents the physical embodiments of one or more systems and/or components described above. For example, the computing device 2200 can represent: the one or more user devices 2102, and/or the server(s) 2104 of FIG. 21. The computing device 2200 can also perform some or each of the blocks in the process 1700, 1800, 1900, 2000 and/or any functionality described herein with respect to FIGS. 1-20. It is understood that the computing device 2200 is not to be construed necessarily as a generic computer that performs generic functions. Rather, the computing device 21200 in some embodiments is a particular machine or special-purpose computer. For example, in some embodiments, the computing device 2200 is or includes: a multi-user mainframe computer system, one or more cloud computing nodes, a single-user system, or a server computer or similar device that has little or no direct user interface, but receives requests from other computer systems (clients), a desktop computer, portable computer, laptop or notebook computer, tablet computer, pocket computer, telephone, smart phone, smart watch, or any other suitable type of electronic device.

Computing device 2200 typically includes a variety of computer-readable media. Computer-readable media can be any available media that can be accessed by computing device 2200 and includes both volatile and nonvolatile media, removable and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 2200. Computer storage media does not comprise signals per se. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

Memory 12 includes computer storage media in the form of volatile and/or nonvolatile memory. The memory may be removable, non-removable, or a combination thereof. Exemplary hardware devices include solid-state memory, hard drives, optical-disc drives, etc. Computing device 2200 includes one or more processors 14 that read data from various entities such as memory 12 or I/O components 20. Presentation component(s) 16 present data indications to a user or other device. Exemplary presentation components include a display device, speaker, printing component, vibrating component, etc.

I/O ports 18 allow computing device 2200 to be logically coupled to other devices including I/O components 20, some of which may be built in. Illustrative components include a microphone, joystick, game pad, satellite dish, scanner, printer, wireless device, etc. The I/O components 20 may provide a natural user interface (NUI) that processes air gestures, voice, or other physiological inputs generated by a user. In some instance, inputs may be transmitted to an appropriate network element for further processing. A NUI may implement any combination of speech recognition, touch and stylus recognition, facial recognition, biometric recognition, gesture recognition both on screen and adjacent to the screen, air gestures, head and eye-tracking, and touch recognition associated with displays on the computing device 2200. The computing device 2200 may be equipped with depth cameras, such as, stereoscopic camera systems, infrared camera systems, RGB camera systems, and combinations of these for gesture detection and recognition. Additionally, the computing device 2200 may be equipped with accelerometers or gyroscopes that enable detection of motion.

As described above, implementations of the present disclosure relate to automatically generating a user interface or rendering one or more applications based on contextual data received about a particular user. The present invention has been described in relation to particular embodiments, which are intended in all respects to be illustrative rather than restrictive. Alternative embodiments will become apparent to those of ordinary skill in the art to which the present invention pertains without departing from its scope.

From the foregoing, it will be seen that this invention is one well adapted to attain all the ends and objects set forth above, together with other advantages which are obvious and inherent to the system and method. It will be understood that certain features and sub combinations are of utility and may be employed without reference to other features and sub combinations. This is contemplated by and is within the scope of the claims.

Accordingly, described herein are various aspects of technology directed to systems and methods for estimating fluid life and condition. It is understood that various features, sub-combinations, and modifications of the embodiments described herein are of utility and may be employed in other embodiments without reference to other features or sub-combinations. Moreover, the order and sequences of steps shown in the example flow diagrams are not meant to limit the scope of the present disclosure in any way, and in fact, the steps may occur in a variety of different sequences within embodiments hereof. Such variations and combinations thereof are also contemplated to be within the scope of embodiments of this disclosure.

In some embodiments, a computerized system, such as the computerized system described in any of the embodiments above, comprises at least one or more processors, and one or more computer storage memories having computer-executable instructions that, when used by the one or more processors, implement a method comprising the following operations—accessing, from a data store in computer memory, first data associated with a plurality of heat transfer fluid samples, each heat transfer fluid sample of the plurality of heat transfer fluid samples being associated with a first plurality of parameters; converting the first data into a first set of feature vectors and mapping the first set of feature vectors in a vector space, each feature vector of the first set of feature vectors representing a corresponding heat transfer fluid sample and the first plurality of parameters; receiving second data associated with a heat transfer fluid; converting the second data into a second feature vector and mapping the second feature vector in the vector space; computing, via one or more machine learning models, a distance, in the vector space, between at least one of the first set of feature vectors and the second feature vector; based at least in part on the computing, estimating at least one of: a fluid life or condition score for the heat transfer fluid; and based at least in part on the computing, generating a user interface element that indicates the at least one of the fluid life or condition score.

Advantageously, these computerized system embodiments, as described herein, provide technical solutions by improving the accuracy of existing hardware-based fluid quality measurement systems and particular computer applications. These computer system embodiments also improve human-computer interaction, user interfaces, and the user experience (e.g., via less drilling, historical graphing, trend lines, and more intuitive user interface features) and computer resource consumption (e.g., memory consumption and CPU bottlenecking) relative to existing technologies, as described herein.

In any combination of the above computerized system embodiments, the first plurality of parameters include at least two parameter values selected from a group of parameter values consisting of: a high boilers value, a base number value, a low boilers value, an opacity value, a color value, a conductivity value, an absorbance value a moisture content value, pH value, an acid number value, an insoluble solids value, a bromine index value, a viscosity value, a carbon residue value, a specific gravity value, a flash point value, a non-evaporable content value, a contamination value, an autoignition temperature value, an operating temperature value, an on-stream time value, and a mixture value.

In any combination of the above computerized system embodiments, the second data includes a second plurality of parameters, and wherein the distance is based at least in part on values of the second plurality of parameters indicated in the second feature vector relative to other values of the first plurality of parameters indicated in another feature vector of the first set of feature vectors.

In any combination of the above computerized system embodiments, the estimating is further based on training the machine learning model using the plurality of heat fluid transfer samples.

In any combination of the above computerized system embodiments, the computer storage memory having computer-executable instructions stored thereon which, when executed by the one or more processors, implement a method further comprising: based at least in part on the at least one of the fluid life or condition score exceeding a threshold, generating a recommendation signal that indicates instructions to service the heat transfer fluid.

In any combination of the above computerized system embodiments, the fluid life is indicative of at least one of: an estimate of life remaining for the heat transfer fluid or an estimate indicating a point in time at which the heat transfer fluid will expire.

In any combination of the above computerized system embodiments, the computer storage memory having computer-executable instructions stored thereon which, when executed by the one or more processors, implement a method further comprising: based on the at least one of the fluid life or condition score exceeding a threshold, generating a recommendation signal that recommends replacement of at least a portion of the heat transfer fluid.

In any combination of the above computerized system embodiments, the computer storage memory having computer-executable instructions stored thereon which, when executed by the one or more processors, implement a method further comprising: generating historical trend data of at least one parameter associated with the heat transfer fluid; and in response to the generating of the historical trend data, causing display of the historical trend data and an identifier that indicates the at least one parameter.

In any combination of the above computerized system embodiments, the computer storage memory having computer-executable instructions stored thereon which, when executed by the one or more processors, implement a method further comprising: prompting a user to obtain a sample kit to cause extraction of at least a portion of the heat transfer fluid in its operating system, and wherein the receiving of the second data occurs based on the prompting.

In some embodiments, a computer-implemented method, such as the computer-implemented method described in any of the embodiments above, comprises the following operations—receiving first data associated with a heat transfer fluid; accessing, from a data store in computer memory, second data associated with a plurality of heat transfer fluid samples, wherein the second data comprises one or more first parameters associated with each of the plurality of heat transfer fluid samples, wherein the second data excludes the first data; using the first data and the second data, generating, via a computer model, at least one of: an estimate of life remaining for the heat transfer fluid or a score that indicates a condition of the heat transfer fluid; and in response to the generating, generating a user interface element that indicates at least one of: the life remaining or the score for the heat transfer fluid.

Advantageously, these computer-implemented method embodiments, as described herein, provide technical solutions by improving the accuracy of existing hardware-based fluid quality measurement systems and particular computer applications. These computer-implemented embodiments also improve human-computer interaction, user interfaces, and the user experience (e.g., via less drilling, historical graphing, trend lines, and more intuitive user interface features) and computer resource consumption (e.g., memory consumption and CPU bottlenecking) relative to existing technologies, as described herein.

In any combination of the above computer-implemented method or computerized system embodiments, at least one of the first data or the one or more first parameters include at least one parameter value selected from a group of parameter values consisting of: a specific gravity value, a color value, a high boilers, a base number value, value, an opacity value, a low boilers value, a pH value, a moisture content value, an absorbance value, an acid number value, a bromine index value, an insoluble solids value, a degradation value, a viscosity value, a carbon residue value, an autoignition temperature value, a flash point value, a non-evaporable content value, a conductivity value, a contamination value, operating temperature value, an on-stream time value, and a mixture value.

In any combination of the above computer-implemented method or computerized system embodiments, the first data includes one or more second parameters, and wherein the one or more second parameters share at least one parameter attribute with the one or more first parameters, and wherein the at least one of the estimate of life remaining or the score is based at least in part on determining whether a value of the one or more second parameters exceeds one or more predetermined thresholds associated with the plurality of heat transfer fluid samples.

In any combination of the above computer-implemented method or computerized system embodiments, the at least one of the estimate or the score is further based on weighting the one or more second parameters based on information derived from the plurality of heat transfer fluid samples.

In any combination of the above computer-implemented method or computerized system embodiments, the first data includes one or more second parameters, the method further comprising: determining whether a value of the one or more second parameters falls within a specified range, wherein the at least one of the estimate of life remaining or the score is generated based at least in part on the determining; and based at least in part on the determining, generating a second user interface element that indicates whether the value of the one or more second parameters falls within the specified range.

In any combination of the above computer-implemented method or computerized system embodiments, the computer model includes at least one of: a machine learning model or a statistical model.

In any combination of the above computer-implemented method or computerized system embodiments, the method further comprising: based at least in part on one or more parameter values of the first data not meeting a threshold, generating a recommendation signal that indicates instructions to service the heat transfer fluid.

In any combination of the above computer-implemented method or computerized system embodiments, the estimate of the life remaining and the score is associated with an ability of the heat transfer fluid to provide indirect transfer of process heat, and wherein the method further comprising: based at least in part on the generating of the recommendation signal, modifying the estimate of the life remaining or the score, the modifying being indicative of what the estimate of the life remaining or the score would be if the service to the heat transfer fluid was performed.

In any combination of the above computer-implemented method or computerized system embodiments, the method further comprising: generating historical trend data of at least one parameter associated with the heat transfer fluid; in response to the generating of the historical trend data, causing display of the historical trend data and an identifier that indicates the at least one parameter at a graph; and causing presentation, at the graph, of an indicator that indicates a point in time at which a particular maintenance action was taken.

In some embodiments, one or more computer storage media, such as the one or more computer storage media described in any of the embodiments above has computer-executable instructions embodied thereon that, when executed, by one or more processors, cause the one or more processors to perform a method, the method comprising the following operations—using first data and second data to generate a fluid life estimate, the first data associated with a heat transfer fluid, the second data accessed from a data store in computer memory, the second data being associated with a plurality of heat transfer fluid samples; and utilizing the fluid life estimate to generate a user interface element that at least partially indicates a life remaining for the heat transfer fluid.

Advantageously, these computer storage media embodiments, as described herein, provide technical solutions by improving the accuracy of existing hardware-based fluid quality measurement systems and particular computer applications. These computer storage media embodiments also improve human-computer interaction, user interfaces, and the user experience (e.g., via less drilling, historical graphing, trend lines, and more intuitive user interface features) and computer resource consumption (e.g., memory consumption and CPU bottlenecking) relative to existing technologies, as described herein.

In any combination of the above embodiments of the one or more computer storage media, the computer-implemented method, or the computerized system, the generation of the fluid life estimate occurs based on using the second data associated with the plurality of heat transfer fluid samples and the first data associated with the heat transfer fluid as inputs into a computer model.

### DEFINITIONS

“And/or” is the inclusive disjunction, also known as the logical disjunction and commonly known as the “inclusive or.” For example, the phrase “A, B, and/or C,” means that at least one of A or B or C is true; and “A, B, and/or C” is only false if each of A and B and C is false.

A “set of” items means there exists one or more items; there must exist at least one item, but there can also be two, three, or more items. A “subset of” items means there exists one or more items within a grouping of items that contain a common characteristic.

A “plurality of” items means there exists more than one item; there must exist at least two items, but there can also be three, four, or more items.

“Includes” and any variants (e.g., including, include, etc.) means, unless explicitly noted otherwise, “includes, but is not necessarily limited to.”

A “user” or a “subscriber” includes, but is not necessarily limited to: (i) a single individual human; (ii) an artificial intelligence entity with sufficient intelligence to act in the place of a single individual human or more than one human; (iii) a business entity for which actions are being taken by a single individual human or more than one human; and/or (iv) a combination of any one or more related “users” or “subscribers” acting as a single “user” or “subscriber.”

The terms “receive,” “provide,” “send,” “input,” “output,” and “report” should not be taken to indicate or imply, unless otherwise explicitly specified: (i) any particular degree of directness with respect to the relationship between an object and a subject; and/or (ii) a presence or absence of a set of intermediate components, intermediate actions, and/or things interposed between an object and a subject.

A “module” or “component” is any set of hardware, firmware, and/or software that operatively works to do a function, without regard to whether the module is: (i) in a single local proximity; (ii) distributed over a wide area; (iii) in a single proximity within a larger piece of software code; (iv) located within a single piece of software code; (v) located in a single storage device, memory, or medium; (vi) mechanically connected; (vii) electrically connected; and/or (viii) connected in data communication. A “sub-module” is a “module” within a “module.”

The terms first (e.g., first cache), second (e.g., second cache), etc. are not to be construed as denoting or implying order or time sequences unless expressly indicated otherwise. Rather, they are to be construed as distinguishing two or more elements. In some embodiments, the two or more elements, although distinguishable, have the same makeup. For example, a first memory and a second memory may indeed be two separate memories but they both may be RAM devices that have the same storage capacity (e.g., 4 GB).

The term “causing” or “cause” means that one or more systems (e.g., computing devices) and/or components (e.g., processors) may in in isolation or in combination with other systems and/or components bring about or help bring about a particular result or effect. For example, a server computing device may “cause” a message to be displayed to a user device (e.g., via transmitting a message to the user device) and/or the same user device may “cause” the same message to be displayed (e.g., via a processor that executes instructions and data in a display memory of the user device). Accordingly, one or both systems may in isolation or together “cause” the effect of displaying a message.

1. A computer-implemented method, comprising: receiving first data associated with a heat transfer fluid; accessing, from a data store in computer memory, second data associated with a plurality of heat transfer fluid samples, wherein the second data comprises one or more first parameters associated with each of the plurality of heat transfer fluid samples, wherein the second data excludes the first data; using the first data and the second data, generating, via a computer model, at least one of: an estimate of life remaining for the heat transfer fluid or a score that indicates a condition of the heat transfer fluid; and in response to the generating, generating a user interface element that indicates at least one of: the life remaining or the score for the heat transfer fluid.

2. The method of claim 1, wherein at least one of the first data or the one or more first parameters include at least one parameter value selected from a group of parameter values consisting of: a specific gravity value, a color value, a high boilers, a base number value, value, an opacity value, a low boilers value, a pH value, a moisture content value, an absorbance value, an acid number value, a bromine index value, an insoluble solids value, a degradation value, a viscosity value, a carbon residue value, an autoignition temperature value, a flash point value, a non-evaporable content value, a conductivity value, a contamination value, an operating temperature value, an on-stream time value, and a mixture value.

3. The method of claim 1, wherein the first data includes one or more second parameters, and wherein the one or more second parameters share at least one parameter attribute with the one or more first parameters, and wherein the at least one of the estimate of life remaining or the score is based at least in part on determining whether a value of the one or more second parameters exceeds one or more predetermined thresholds associated with the plurality of heat transfer fluid samples.

4. The method of claim 3, wherein the at least one of the estimate or the score is further based on weighting the one or more second parameters based on information derived from the plurality of heat transfer fluid samples.

5. The method of claim 1, wherein the first data includes one or more second parameters, the method further comprising: determining whether a value of the one or more second parameters falls within a specified range, wherein the at least one of the estimate of life remaining or the score is generated based at least in part on the determining; and based at least in part on the determining, generating a second user interface element that indicates whether the value of the one or more second parameters falls within the specified range.

6. The method of claim 1, wherein the computer model includes at least one of: a machine learning model or a statistical model.

7. The method of claim 1, further comprising: based at least in part on one or more parameter values of the first data not meeting a threshold, generating a recommendation signal that indicates instructions to service the heat transfer fluid.

8. The method of claim 7, wherein the estimate of the life remaining and the score is associated with an ability of the heat transfer fluid to provide indirect transfer of process heat, and wherein the method further comprising: based at least in part on the generating of the recommendation signal, modifying the estimate of the life remaining or the score, the modifying being indicative of what the estimate of the life remaining or the score would be if the service to the heat transfer fluid was performed.

9. The method of claim 8, further comprising: generating historical trend data of at least one parameter associated with the heat transfer fluid; in response to the generating of the historical trend data, causing display of the historical trend data and an identifier that indicates the at least one parameter at a graph; and causing presentation, at the graph, of an indicator that indicates a point in time at which a particular maintenance action was taken.

10. One or more computer storage media having computer-executable instructions embodied thereon that, when executed, by one or more processors, cause the one or more processors to perform a method, the method comprising: using first data and second data to generate a fluid life estimate, the first data associated with a heat transfer fluid, the second data accessed from a data store in computer memory, the second data being associated with a plurality of heat transfer fluid samples; and utilizing the fluid life estimate to generate a user interface element that at least partially indicates a life remaining for the heat transfer fluid.

11. The one or more computer storage media of claim 10, wherein generation of the fluid life estimate occurs based on using the second data associated with the plurality of heat transfer fluid samples and the first data associated with the heat transfer fluid as inputs into a computer model.

12. A computerized system, comprising: one or more processors; and computer storage memory having computer-executable instructions stored thereon which, when executed by the one or more processors, implement a method comprising: accessing, from a data store in computer memory, first data associated with a plurality of heat transfer fluid samples, each heat transfer fluid sample of the plurality of heat transfer fluid samples being associated with a first plurality of parameters; converting the first data into a first set of feature vectors and mapping the first set of feature vectors in a vector space, each feature vector of the first set of feature vectors representing a corresponding heat transfer fluid sample and the first plurality of parameters; receiving second data associated with a heat transfer fluid; converting the second data into a second feature vector and mapping the second feature vector in the vector space; computing, via one or more machine learning models, a distance, in the vector space, between at least one of the first set of feature vectors and the second feature vector; based at least in part on the computing, estimating at least one of: a fluid life or condition score for the heat transfer fluid; and based at least in part on the computing, generating a user interface element that indicates the at least one of the fluid life or condition score.

13. The system of claim 12, wherein the first plurality of parameters include at least two parameter values selected from a group of parameter values consisting of: a high boilers value, a base number value, a low boilers value, an opacity value, a color value, a conductivity value, an absorbance value a moisture content value, pH value, an acid number value, an insoluble solids value, a bromine index value, a viscosity value, a carbon residue value, a specific gravity value, a flash point value, a non-evaporable content value, a contamination value, an autoignition temperature value, an operating temperature value, an on-stream time value, and a mixture value.

14. The system of claim 12, wherein the second data includes a second plurality of parameters, and wherein the distance is based at least in part on values of the second plurality of parameters indicated in the second feature vector relative to other values of the first plurality of parameters indicated in another feature vector of the first set of feature vectors.

15. The system of claim 12, wherein the estimating is further based on training the machine learning model using the plurality of heat fluid transfer samples.

16. The system of claim 12, wherein the computer storage memory having computer-executable instructions stored thereon which, when executed by the one or more processors, implement a method further comprising: based at least in part on the at least one of the fluid life or condition score exceeding a threshold, generating a recommendation signal that indicates instructions to service the heat transfer fluid.

17. The system of claim 12, wherein the fluid life is indicative of at least one of: an estimate of life remaining for the heat transfer fluid or an estimate indicating a point in time at which the heat transfer fluid will expire.

18. The system of claim 12, wherein the computer storage memory having computer-executable instructions stored thereon which, when executed by the one or more processors, implement a method further comprising: based on the at least one of the fluid life or condition score exceeding a threshold, generating a recommendation signal that recommends replacement of at least a portion of the heat transfer fluid.

19. The system of claim 12, wherein the computer storage memory having computer-executable instructions stored thereon which, when executed by the one or more processors, implement a method further comprising: generating historical trend data of at least one parameter associated with the heat transfer fluid; and in response to the generating of the historical trend data, causing display of the historical trend data and an identifier that indicates the at least one parameter.

20. The system of claim 19, wherein the computer storage memory having computer-executable instructions stored thereon which, when executed by the one or more processors, implement a method further comprising: prompting a user to obtain a sample kit to cause extraction of at least a portion of the heat transfer fluid in its operating system, and wherein the receiving of the second data occurs based on the prompting.
